(19) United States
(12) Patent Application Publication
(10) Pub. No.: US 2018/0184735 A1
(43) Pub. Date: Jul. 5, 2018

(54) PHYSIOLOGICAL MONITORING GARMENTS WITH ENHANCED SENSOR STABILIZATION

(71) Applicants: Gianluigi LONGINOTTI-BUITONI, Haute-Nendaz (CH); Andrea ALIVERTI, Como (IT)

(72) Inventors: Gianluigi LONGINOTTI-BUITONI, Haute-Nendaz (CH); Andrea ALIVERTI, Como (IT)

(21) Appl. No.: 15/905,811

(22) Filed: Feb. 26, 2018

Related U.S. Application Data
(63) Continuation-in-part of application No. PCT/IB2016/001319, filed on Aug. 24, 2016.
(60) Provisional application No. 62/209,034, filed on Aug. 24, 2015.
(60) Provisional application No. 62/233,693, filed on Sep. 28, 2015.

(51) Int. Cl. A41D 13/12; A41B 1/08; A41D 1/00; A41D 1/06; A63B 24/00; A61B 5/0408; A61B 5/00; A61B 5/11

(52) U.S. Cl. CPC A41D 13/1281; A41B 1/08; A41D 1/005; A41D 1/06; A61B 2562/0209; A61B 5/04085; A61B 5/6805; A61B 5/1123; A63B 2230/04; A63B 24/0062

(57) ABSTRACT

Described herein are apparatuses (e.g., garments, including but not limited to shirts, pants, and the like) for detecting and monitoring physiological parameters, such as respiration, cardiac parameters, and the like that include individual skin contact-enhancing expandable elements.

### CROSS REFERENCE TO RELATED APPLICATIONS

This patent application claims priority as a continuation-in-part to International Patent Application No. PCT/IB2016/001319, titled “PHYSIOLOGICAL MONITORING GARMENTS WITH ENHANCED SENSOR STABILIZATION,” filed on Aug. 24, 2016, now International Patent Application Publication No. WO 2017/033058, which claims priority to U.S. Provisional Patent Application No. 62/209,034, titled “PHYSIOLOGICAL MONITORING GARMENTS WITH ENHANCED SENSOR STABILIZATION,” filed on Aug. 24, 2015, and U.S. Provisional Patent Application No. 62/233,693, titled “PHYSIOLOGICAL MONITORING GARMENTS WITH ENHANCED SENSOR STABILIZATION,” filed on Sep. 29, 2015, each of which are herein incorporated by reference in its entirety.

This application may be related to U.S. patent application Ser. No. 15/877378, titled “FLEXIBLE FABRIC RIBBON CONNECTORS FOR GARMENTS WITH SENSORS AND ELECTRONICS,” filed on Jan. 22, 2018, and to U.S. patent application Ser. No. 14/644,180, filed on Mar. 10, 2015 and titled “PHYSIOLOGICAL MONITORING GARMENTS,” which claims priority to U.S. Provisional Patent Application No. 61/950,782, filed Mar. 10, 2014, titled “PHYSIOLOGICAL MONITORING GARMENTS,” U.S. Provisional Patent Application No. 62/058,519, filed Oct. 1, 2014, titled “DEVICES AND METHODS OF RUSE WITH PHYSIOLOGICAL MONITORING GARMENTS,” U.S. Provisional Patent Application No. 62/080,966, filed Nov. 17, 2014, titled “PHYSIOLOGICAL MONITORING GARMENTS,” and U.S. Provisional Patent Application No. 62/097,560, filed Dec. 29, 2014, titled “STRETCHABLE, CONDUCTIVE TRACES AND METHODS OF MAKING AND USING SAME,” each of which is herein incorporated by reference in its entirety.

This patent application may also be related to U.S. patent application Ser. No. 14/612,060, filed Feb. 2, 2015, titled “GARMENTS HAVING STRETCHABLE AND CONDUCTIVE INK,” which is a continuation of U.S. patent application Ser. No. 14/331,185, filed Jul. 14, 2014, and titled “METHODS OF MAKING GARMENTS HAVING STRETCHABLE AND CONDUCTIVE INK,” now U.S. Pat. No. 8,945,328 each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are wearable apparatuses (e.g., “garments”) that can reliably and continuously detect skin electrical signals from a user (e.g. from a wearable electronics based garment worn by the user) even while the user is moving. These devices may be configured for both secure and comfortable wear. For example, described herein are wearable monitoring and input systems that can monitor physiological parameters from the wearer using regionally expandable supports to comfortably and reliably hold the sensor against the subject's skin.

### BACKGROUND

In the last twenty years, clothing that includes sensors have been suggested. See, e.g., US2007/0178716 to Glaser et al., which describes a “modular microelectronic-system” designed for use with wearable electronics. US2012/0071039 to Debock et al. describes interconnect and termination methodology fore-textiles that include a “conductive layer that includes conductors includes a terminal and a base separately provided from the terminal. The terminal has a mating end and a mounting end.” US2005/0029680 to Jung et al. describes a method and apparatus for the integration of electronics in textiles.

For example, cardiovascular and other health-related problems, including respiratory problems may be detected by monitoring a patient. Monitoring may allow early and effective intervention, and medical assistance may be obtained based on monitored physiological characteristics before a particular health issue becomes fatal. Unfortunately, most currently available cardiovascular and other types of health monitoring systems are cumbersome and inconvenient (e.g., impractical for everyday use) and in particular, are difficult or impractical to use for long-term monitoring, particularly in an unobtrusive manner

It has been proposed that patient health parameters, including vital signs (such as ECG, respiration, blood oxygenation, heart rate, etc.) could be actively monitoring using one or more wearable monitors, however, to date such monitors have proven difficult to use and relatively inaccurate. Ideally such monitors could be unobtrusively worn by the subject (e.g., as part of a garment, jewelry, or the like). Although such garments have been proposed, see, e.g., US 2012/0136231, these garments suffer from a number of deficits, including being uncomfortable, difficult to use, and providing inaccurate results. For example, in applications such as US 2012/0136231, a number of individual electrodes are positioned on the garment and connected to a processor by woven conductive fibers or the like; although such garments “require . . . consistent and firm conductive contact with the subject's skin,” in order to provide accurate readings, such designs require that the garment be restrictive in order to prevent movement of the garment (and thus sensors) contacting these skin regions. Such a configuration rapidly becomes uncomfortable, particularly in a garment that would ideally be worn for many hours or even days. In addition, even such tightly worn garments often move relative to the wearer (e.g., slip or ride up). Further, devices/garments such as those described in the prior art are difficult and expensive to manufacture, and are often rather “fragile”, preventing robust usage and washing. Finally, such devices/garments typically do not allow processing of manual user input directly on the garment, but either relay entirely on passive monitoring, or require an interface of some sort (including off-garment interfaces).

The use of garments including one or more sensors that may sense biometric data have not found widespread use. In part, this may be because such garments may be limited in the kinds and versatility of the inputs that they accept, as well as limits in the comfort, and form factor of the garment. For example, sensors, and the leads providing power to and receiving signals from the sensors have not been fully integrated with the garment in a way that allows the garment to be flexible, attractive, practical, and above all, comfortable. For example, most such proposed garments have not been sufficiently stretchable. Finally, such proposed garments are also limited in the kind of data that they can receive, and how they process the received information.

Thus, existing garments (e.g., devices and wearable sensing apparatuses) and processes for analyzing and communicating the physical and emotional status of an individual may be inaccurate, inadequate, limited in scope, unpleasant, and/or cumbersome.

What is needed are apparatuses (including garments) having one more sensors that may be comfortably worn, yet provide relatively accurate and movement-insensitive measurements over a sustained period of time. It would also be beneficial to provide garments that can be easily and inexpensively manufactured. Finally it may be beneficial to provide garments having a direct user interface that is on the garment, and particularly interfaces which are formed as part of the garment (including the fabric).

In particular, what is needed are comfortable garments that include multiple (discrete) electrodes across the garment that can be held against the skin for reliable measurement of electrical signals. The apparatuses (devices, systems, and particularly garments) described herein may address some or all of the problems identified above.

### SUMMARY OF THE DISCLOSURE

In general, described herein are fabric garments, such as shirts, that cover a wearer's torso and include one or more electrode (electrical sensor) on an inner surface of the garment for sensing a signal (e.g., electrical signal, chemical signal, etc.) from the wearer's body; the sensors are held against the body by a support mechanism that can be expanded in a direction perpendicular to the sensor/inner surface of the garment, where the support is backed (near an outer surface of the garment) by a supporting structure (e.g., fabric, brace, wrap, etc.) that is less flexible than the inner fabric of the garment, so that expanding the support puts the sensor in contact with the body. The support may be configured to expand in one direction (the direction perpendicular to the sensor and the wearer's body) but not a direction parallel to the skin of the person's body (e.g., the sensor and/or inner fabric of the garment). For example, the support may be an inflatable bladder that has manifold (e.g., accordion) walls allowing it to expand and collapse in a predefined direction (e.g., perpendicular to the sensor) when inflated/deflated.

For example, described herein are garment system for monitoring electrical signals from a wearer's skin, the garment system comprising: a garment body formed of a flexible first fabric, wherein the garment body is configured to be worn on a torso; a set of electrical sensors arranged at discrete locations on the garment body when the garment is worn, wherein each electrical sensor comprises a flexible conductive ink electrode printed on an inner surface of the garment body; a second fabric configured to be worn over the first set of electrical sensors, wherein the second fabric is less flexible than the first fabric; a plurality of expandable supports, wherein each expandable support is sandwiched between the second fabric and one of the electrical sensors of the first set of electrical sensors, further wherein each expandable support expands and contracts in an axis perpendicular to the electrical sensors but does not expand in an axis parallel to the electrical sensors; and an expansion control line connecting each of the expandable supports, wherein the expansion control line regulates expansion and contraction of the plurality of expandable supports. By “less flexible” the second fabric may be stiffer and/or less compliant. The second fabric material may be thicker or may include reinforcing stitching and/or adhesive that limits its compliance.

A garment system may be a single, unitary garment (e.g., shirt, sweater, pants, unitard, etc.) or it may be a collection of garments that are specifically adapted to be worn together, such as an undershirt with one or more of a harness, overshirt, bra, etc.). In general, a garment is an article of clothing to be worn by a wearer (user, person, etc.). Garments may include: shirts, undershirts, bras, tops, bottoms, pants, coat, corset, blouse, shorts, gloves, trousers, socks, stockings, tights, or the like. The garment body typically includes the material contacting the body (e.g., the inner surface of the garment) and may include a front panel, a back panel, one or more side panels (optionally), an upper region, a midriff region, a lower region, one or more sleeves (long or short), etc.

The flexible first fabric may be any fabric, particularly those that are comfortable for wearing against the skin. Examples of such fabrics may include (but are not limited to): compression fabrics (e.g., nylon/cotton blends, polyester/cotton blends, Lycra, and other synthetic fibers) or generally flexible fabrics. For example in some variations the fabric comprises a mixture of fabrics, such as a mixture of a synthetic (e.g., polyester) and another material (e.g. Lycra or elastin), e.g., around 25-40% of elastin or Lycra with the remainder being polyester. The second fabric forming the outer supporting portion of the garment system may be the same fabric (e.g., a Lycra or Lycra blend, polyester or polyester blend, cotton or cotton blend, etc.). The second fabric is generally less flexible and more supportive than the first fabric. The second fabric may be reinforced to have less flexibility than the first fabric. For example, the second fabric may include one or more reinforcing supports, such as wires, struts, mesh, or other rigid or semi-rigid members formed, e.g., of a metal, plastic, thread, yarn, etc. so that the second fabric has less give and/or is less deformable than the first fabric, directing the force of the expandable support inwardly, against the sensor and/or skin of the wearer, when expanded.

Although apparatuses including only one sensor are envisioned herein, in general, these apparatuses may have a plurality (e.g., a set or collection) of sensors. In general, these sensors may be electrical sensors (e.g., electrodes, surface electrodes, and particularly ‘dry’ electrodes that do not require a conductive gel). The inventions described herein may include or may work with any skin-contacting sensor, and are not limited to electrical sensors. Other non-electrical sensors that may be used include chemical sensors (e.g., pH sensors), optical sensors (IR sensors, etc.), and the like.

In a particular example, the electrodes described herein may themselves be flexible, bendable and/or stretchable. For example, the electrodes may be formed of a conductive ink that is patterned onto the garment (e.g., by silk-screening, deposition, etc.). Conductive ink traces are described in greater detail herein, but typically may be stretched, bent and/or deformed (including restorably deformed), as the wearer moves. Because this may otherwise make it difficult for the electrodes to maintain reliable contact with the skin when wearing a garment having such an electrode, the inventions described herein to aid in holding the sensor against the skin may be particularly well suited to the applications described herein.

As will be described in greater detail, the sensors (e.g., electrical sensors) on the body may be positioned at various discrete locations on the garment body. These locations may be separate and/or distinct from each other, so that each sensor is positioned in a separate and distinct location for contacting a wearer's skin. For example, for ECG electrodes, as described below, although the electrodes may be connected by one or more electrical conductors (traces or lines), the sensors (electrical sensors) are themselves separate in where they contact the skin, so that they measure a signal from a particular location of the wearer's body, including the predefined V1, V2, V3, V4, V5, V6, etc. lead placement locations. Each sensor may be connected to a separate expandable support, or in some variations a single expandable support may operate on more than one sensor (e.g., covering two or more contact electrodes, etc.).

Expandable supports are typically configured to be held between the sensor (e.g., electrode) present on an inner, wearer-facing surface of the garment, and the outer, less flexible second fabric. The second fabric may be referred to as a backing, backstop, holder, or the like. The second fabric and expandable support may be integrated together. In some variations the expandable support and second fabric are integrated into the garment body (e.g., affixed to the garment body), including over the sensor(s). Alternatively in some variations the expandable support and/or the second fabric (e.g., backing) are separate and/or detachable from the garment body, though configured to be worn atop it. For example, the second fabric may be configured as part of an overgarment, such as an over shirt, harness, halter, bra, or other garment. This overgarment may include aligners to align with the garment body of the undergarment so that the sensors, expandable supports and backing are aligned (and remain in alignment) when worn by the wearer. Aligners may include tabs, attachments (e.g., snaps, buttons, Velcro, etc.), or the like that mate with aligner mates. Aligners may be present on the overgarment or the garment body.

In general, an expandable support may include an expandable member that is configured to expand in one direction, e.g., perpendicular to the sensor's sensing surface when worn, but not in a direction parallel to the sensor's surface (e.g., perpendicular to the direction of expansion). The expandable support may be inflatable, and may be connected, for example, by an expansion control line to a pump. As will be described and illustrated below, the expandable supports may include an expandable body, which may be bladder-like, and allow for expansion by the application of fluid (e.g., air) pressure. The body may be formed of any appropriate material, including rubber, plastic, etc. and may be shaped to control the direction of expansion. For example, the expandable member may have a folded, e.g., accordion-like, body that is configured to expand and contract in the axis perpendicular to the electrical sensors.

A single expansion control line or multiple expansion control lines may be used, and may be connected to one or more pumps. The pump may also be integrated into the garment system, including integrated into the garment body or overgarment, or it may be separate. The support line may be a fluid line adapted to apply fluid pressure, such as air, or liquid (e.g., saline, water, etc.), or the like, to expand the expandable supports.

In some variations, the systems are configured to allow measurement of ECG signals. For example, the garment system may include a set of electrodes configured to extend in a line across the chest (corresponding to the V1-V6 lead positions). In some variations, as described in greater detail below, a second set of electrodes arranged on the garment body adjacent to the first set of electrical sensors (allowing redundancy and/or signal averaging). The garment system configured to sense ECG signals may also include a right arm electrode formed from conductive ink printed on an inner surface of the garment body and/or a left arm electrode formed from conductive ink printed on an inner surface of the garment body, and/or a leg electrode.

In general, the electrical sensors may be connected to a sensor module interface configured to communicate data from the electrical sensors to a sensor manager unit.

As mentioned above, the second fabric may be integrated into the garment body, and/or a second (e.g., overgarment) adapted to be worn atop the garment body, such as a harness. The harness may be a bra or other supportive device, and may include one or more straps, including elastic straps. The second fabric may be rigid or semi-rigid and/or attached to rigid or semi-rigid regions, including having rigid regions connected by elastic regions.

As will be discussed and illustrated below, the sensors may be stretchable and/or flexible, and may be formed of a conductive ink. For example, the electrical sensors described herein may include: a layer of adhesive; a layer of conductive ink having: between about 40-60% conductive particles, between about 30-50% binder; between about 3-7% solvent; and between about 3-7% thickener; and a gradient region between the conductive ink and the adhesive, the gradient region comprising a nonhomogeneous mixture of the conductive ink and the adhesive. In some variations the concentration of conductive ink decreases from a region closer to the layer of conductive ink to the layer of elastic adhesive.

The electrical sensors may be connected to the sensor module interface via a stitched zig-zag connector formed on a separate piece of compression fabric attached to the garment body.

Any of the garment systems described herein may include additional sensors that are not connected to an expandable support. For example, in some variations, the garment system includes at least one stretch sensors (e.g., a respiratory sensor comprising an elastic ribbon impregnated with a conductive ink, an electrical connector at each end of the elastic ribbon, and a cover comprising a piece of compression fabric). In some variations, a stretch sensor may be used with an expandable support.

A garment system for monitoring electrical signals from a wearer's skin, wherein the electrical signals comprise the wearer's electrocardiogram (ECG), may include: a garment body formed of a compression fabric, wherein the garment body is configured to be worn on a torso; a set of electrical sensors arranged on the garment body at discrete locations extending across the left pectoral region of the wearer's chest when the garment is worn, wherein each electrical sensor comprises a flexible conductive ink electrode printed on an inner surface of the garment body; a harness comprising a second fabric configured to be worn over the first set of electrical sensors, wherein the second fabric is less flexible than the first fabric; a plurality of discrete expandable supports, wherein each expandable support is inflatable and sandwiched between the second fabric and one of the electrical sensors of the first set of electrical sensors, further wherein each expandable support comprises an accordion body configured to expand and contract in an axis perpendicular to the electrical sensors not to expand in an axis parallel to the electrical sensors; and an expansion control line connecting each of the expandable supports, wherein the expansion control line comprises an inflation line configured to apply pressure to regulate the expansion and contraction of the plurality of expandable supports.

In some examples, a garment system for monitoring at least one physiological parameter of a wearer, the garment system includes: an outer support structure comprising a compression fabric configured to compress against a torso or a part of the torso when worn; at least one electrode on a region of the wearer covered by the outer support structure wherein the at least one electrode contact the wearer's skin; at least one inflatable member between the support structure and the at least one electrode, wherein the at least one inflatable member is configured to inflate and deflate; and a compression layer situated between the at least one inflatable member and the at least on electrode, wherein the compression layer conforms to the contours of the at least one electrode and the wearer's skin to ensure good electrical connectivity between the wearer's skin and the at least one electrode.

As mentioned above, in some variations, the at least one inflatable member can only expand along a single axis/direction, e.g., perpendicular to the at least one electrode and the support structure. Alternatively, in some variations, the inflatable member can expand multi-directionally.

The inflatable members may be part of an array of inflatable members designed to press the electrode(s) against the skin of the wearer. The array of inflatable members may be in fluid connection with each other and/or with a pump. The pump may be integrated into the garment system.

Any of the garments described herein may be configured as extended-wear garments. For example, described herein are extended-wear monitoring garments that may be used to monitor and detect various physiological parameters of a wearer. These wearable devices may be worn for extended periods of time without the need for laundering. These extended-wear monitoring garments are able to monitor, record, and/or send the wearer's physiological parameters for later analyses. The communication garments may also detect and respond to signals from the user (e.g. from a wearable “intelligent” garment) and that can communicate with the user (and/or others) and may perform other useful functions. For example, such a communication platform may be configured to accurately detect, process, compare, transfer and communicate, in real time, physiological signals of the wearer (such as a person, an animal, etc.). A wearable communication platform may include an intelligent garment that is a wearable item that has one or more sensors (such as for sensing a condition of a user) and that is capable of interacting with another component(s) of an intelligent apparel platform to create a communication or other response or functionality based on the sense obtained by the sensor.

The extended-wear monitoring garments described herein are sartorial communication devices, such that they continuously conform to the wearer's body. The term “continuously conform” can mean that the material conforms and contacts the skin surface of a wearer. While adequate contact between any sensors or monitoring units on the extended-wear monitoring garment, the extended-wear monitoring garment need to be overly tight, but may be biased against the skin over all or a majority of the garment. Continuously conforming may refer to the sensor-containing regions of the garment that contacts the wearer's skin when worn, even as the wearer moves about.

The term “physiological parameters” may refer to any value indicating the physiological status of the wearer. Physiological parameters can include vital signs, autonomic responses, and so forth.

The term “body sensor” are sensors that generally determine information about the wearer without requiring the wearer's conscious input. A body sensor can detect a physiological parameter, including vital signs (pulse/heart rate, blood pressure, body temperature, galvanic skin response (e.g. sweat), and so forth. A body sensor can also be used to detect a wearer's motion or movements, such as when a wearer is running or standing still.

The sensing components of the extended-wear monitoring garment can also be manually activated and controlled. Manual control can be by way of volitional touch with the wearer's hand or possibly some other appendage. Volitional touch generally indicates that the wearer consciously performed the action. For example, the wearer can touch a sensor to obtain a physiological parameter that he is interested in knowing at a particular time or particular scenario. It may also be possible to incorporate into the extended-wear monitoring garment a means for activating or controlling via voice command

The sensing components of the extended-wear monitoring garment may either be integrated into the extended-wear monitoring garment or the monitoring components may be removed in part or in whole. In the former case, it would especially desirable to minimize the need for washing/laundering the extended-wear monitoring garment, such that the sensing components are minimally exposed to damaging moisture and cleaning agents. In the latter case, where it may be possible to remove the sensors or a portion of the sensor module prior to laundering, this would help to extend the life of the extended-wear monitoring garment. In this latter case, it would still be preferable to extend the wear-ability of the extended-wear monitoring garment because some electrical component, such as the conductive ink traces, remain integrated in the extended-wear monitoring garment.

In order to minimize the amount of laundering required of an extended-wear monitoring garment that can be worn regularly, the extended-wear monitoring garment has been designed to include cut-outs in the areas of the extended-wear monitoring garment that correspond to parts of the body that produce the most sweat and does not easily dry quickly due to its location/proximity to other body parts. Having these areas on the wearer free from constraint, helps increase air flow and circulation to these areas. Also, the extended-wear monitoring garment cut-outs reduce the amount of sweat deposited and the amount of bacterial growth that is transferred onto the extended-wear monitoring garment. Exposing less of the extended-wear monitoring garment to sweat, protein, and bacteria, in turn, helps to minimize the odors transferred to the extended-wear monitoring garment when worn over an extended period of time and thus minimizes the need for frequent laundering of the extended-wear monitoring garment.

The present extended-wear monitoring garment may include body sensors, conductive traces, and/or interactive sensors are configured to withstand immersion in water. Thus, in general, the wearable communication platforms described herein may be washed (e.g., washed in water).

In some instances, the extended-wear monitoring garment contains compressive material. The extended-wear monitoring garment may comprise a compression garment that is configured to continuously conform to a wearer's body when the garment is worn. In some cases, compressive material corresponds to areas on the wearer's body where a detectable change (such as girth or expansion and contraction of a certain amount) can be correlated to a physiological parameter. In some embodiments, the flexible garment includes a first axis and a second axis perpendicular to the first axis wherein the garment is configured to change in size along the first axis and to substantially maintain a size along the second axis.

In some embodiments, the body sensor is in electrical contact with the skin of the individual. In some embodiments, the sensor includes one of an accelerometer, an electrocardiogram (ECG) sensor, an electroencephalography sensor (EEG), and a respiratory sensor. In some embodiments, the body sensor includes a first sensor, and the garment further includes a second sensor configured to sense one of a wearer's position, movement, and/or physiological status, and thereby generate a second body sensor signal. In some embodiments, the conductive trace is configured to conform to the user's body when the flexible garment is worn by the user. In some embodiments, the conductive trace is on a surface of the garment. In some embodiments, the flexible garment further includes a seam enclosing the conductive trace.

In some embodiments, the interactive sensor is configured to transmit a first interactive sensor signal when the user's hand activates the interactive sensor once and to transmit a second interactive sensor signal when the user's hand activates the interactive sensor twice in succession wherein the first interactive sensor signal is different from the second interactive sensor signal. In some such embodiments, the flexible garment further includes a plurality of interactive sensors wherein the first interactive sensor is configured to send a first interactive sensor signal and the second interactive sensor is configured to send a second interactive sensor signal which is different from the first interactive sensor signal. In some of these embodiments, the interactive sensors are on a front of the garment.

The extended-wear monitoring garment may be flexible, compressive, and configured to continuously conform to a wearer's body when worn. The extended-wear monitoring garment may be configured to move with a wearer's body. A body sensor may be, for example, a printed sensor or a physical sensor and may be sufficiently flexible or extensible in at least one direction in order to maintain the flexibility of the shirt. A body sensor may be, for example an accelerometer, a gyroscope, a magnetoscope, and may detect, for example, a wearer's respiratory rate, heart rate, skin conductivity, movement, position in space, inspiratory time, expiratory time, tidal volume, perspiration, pulse, moisture, humidity, elongation, stress, glucose level, pH, resistance, motion, temperature, impact, speed, cadence, proximity, flexibility, movement, velocity, acceleration, posture, relative motion between limbs and trunk, location, responses to transdermal activation, electrical activity of the brain, electrical activity of muscles, arterial oxygen saturation, muscle oxygenation, oxyhemoglobin concentration, deoxyhemoglobin concentration, etc. A sensor module may be configured for managing and controlling power, body sensors, memory, external data, interactive sensors, body “expressions”, feedback, transdermal control processes, module enhancements, social media, software development, etc. An interactive sensor (“touchpoint”) may be activated by touching or by relative proximity of a user's hand or other item (even though one or more layer of clothing).

A wearable, flexible extended-wear monitoring garment may include: a body sensor on the garment configured to sense one of a wearer's position, a wearer's movement, and a wearer's physiological status and thereby generate a body sensor signal; a conductive trace on the garment, connected with the sensor and configured to communicate the body sensor signal from the sensor to a sensor module for analysis; an interactive sensor on the garment configured to transmit an interactive sensor signal to the sensor module when the wearer's hand activates the interactive sensor wherein the sensor module is configured to control an audio output and/or a visual output in response to the interactive sensor signal; and a sensor module for receiving the body sensor signal from the body sensor, processing the signal to generate an output signal, and outputting the output signal to thereby provide a feedback output. The wearable, flexible extended-wear monitoring garment may be configured to continuously conform to a wearer's body when the flexible garment is worn by the wearer. In some embodiments, the garment is configured to be worn on the wearer's torso.

In any of the monitoring garments described herein, the apparatus may also include a plurality of surface regions on the garment, wherein each surface region corresponds to a contact surface for one of the interactive sensors. Each of the plurality of surface regions may comprise a visual marker on the fabric of the garment indicating the location of the interactive sensor corresponding to the surface region. For example, each surface region corresponding to a touch point (interactive sensor) contact surface may be marked by a color, icon, or the like. In some variations, the contact surface includes a tactile marker, such as a textured or raised region. The contact surface of an interactive sensor may be any appropriate size. For example, a contact surface for an interactive sensor may be between about 10 mm and about 150 mm in diameter. In general, an interactive sensor (also referred to as a touchpoint sensor) may be configured so that it can only be activated by contact with the outwardly-facing side of the sensor (e.g., the side of the sensor that faced away from the body when the garment is worn).

The extended-wear monitoring garments also include a control module. The control module can manage the sensors input and outputs directly. The control module may communicate with external devices that are able to retain and analyze the outputs from the sensors for a particular wearer. The control module is also able to process both manual and automated inputs from a wearer and deliver the requisite response.

Finally, in cases where there is more than one sensor, the extended-wear monitoring garment may include a sensor management module or system. The sensor management module can help manage individual sensors or can aid in the interaction between different sensing components. Further, the sensor management module can be used to retain input gathered from the various sensors and communicate with the control module.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows a front view of a shirt configured as a respiration monitoring garment.

FIG. 1B is a partial view of the front and lateral regions of the shirt of FIG. 1A.

FIG. 1C shows a back view of the same garment of FIGS. 1A and 1B.

FIGS. 1D, 1E and 1F show front, front and lateral, and back views, respectively of another garment configured to measure regional respiration, similar to the garment shown in FIGS. 1A-1C.

FIG. 2A is a front view of a garment (showing both a shirt and pants) for measuring an ECG.

FIG. 2B is a back view of the garment (shirt and pants) of FIG. 2A.

FIG. 3A is a front view of another variation of a garment for measuring ECG in which the limb leads are positioned on the shirt, e.g., not requiring leg leads. For example, when exercise stress tests are performed, limb leads are often placed on the trunk to avoid artifacts while ambulatory (arm leads moved subclavicularly, and leg leads medial to and above the iliac crest).

FIG. 3B is a back view of the garment of FIG. 3A.

FIGS. 4A and 4B show front and back views, respectively, of a garment configured to detect ECGs.

FIG. 5 is a graph characterizing the force vs. extension for one variation of stretchable conductive ink.

FIG. 6 is a graph characterizing the resistance for one variation of stretchable conductive ink.

FIGS. 7A, 7B and 7C show front, side and back views, respectively of a garment configured to be worn during sleep to monitor a subject's sleep.

FIGS. 8A and 8B show a front and back view of a collar that may be included.

FIG. 9A shows one example of a portion of a garment system including an expandable support structure, shown in a collapsed (e.g., deflated) state.

FIG. 9B shows the expandable support of FIG. 9A an expended (e.g., inflated) state applying pressure to ensure proper contact between the electrode and the wearer's skin.

FIG. 9C is a schematic showing a close-up view of an expandable support such as the one shown in FIGS. 9A and 9B.

FIG. 10 is a schematic illustration of a lateral slice through a torso at the level of a set of electrical sensors (electrodes) positioned as V1-V6 ECG leads also showing individual expandable supports expanded to help secure the electrodes against the wearer's body.

FIG. 11A shows one example of an outer garment configured as a harness to be worn over an undergarment having flexible sensors in predefined locations; in this example the expandable supports are incorporated together with a second, less flexible backing material that directs the expansion of the expandable supports toward the wearer's body and against any sensors beneath them.

FIG. 11B shows a plurality of the expandable support (stabilization) structures in which each expandable support member is in contact with a corresponding electrode. This arrangement may be particularly helpful in stabilizing ECG electrodes.

FIGS. 11C and 11D show front and side views, respectively, of a set of expandable (e.g., inflatable) support structures connected by a common expansion control line connecting each of the expandable supports.

FIG. 12A shows another view of the expandable support structures (similar to FIG. 11B), configured as inflatable support structures. FIG. 12A illustrates the inflation these support structures thorough a common expansion control line connected to a hand pump.

FIG. 12B shows an enlarged view of a connector (expansion control line) that fluidly connects a plurality of the expandable supports with each other and a pump for increasing the pressure to drive expansion of the expandable supports.

FIGS. 13A and 13B show front and back views, respectively, of a garment.

FIGS. 14A and 14B illustrate front and back views, respectively of a garment with a support garment (e.g., harness).

FIGS. 14C and 14D illustrate a front and back view, respectively of a support garment that can be used with the garments described herein.

FIGS. 15A and 15B illustrate an inflatable support device in accordance with some embodiments.

FIG. 15C illustrates the inflatable support device relative to a female chest.

FIGS. 16A and 16B illustrate a front and back view, respectively of a support garment that can be used with the garments described herein.

FIGS. 17A and 17B illustrate an inflatable support device in accordance with some embodiments.

FIG. 17C illustrates the inflatable support device relative to a male chest.

FIGS. 18A and 18B illustrate front and back views, respectively of a garment with a support garment.

FIGS. 18C-18E illustrate one system including a garment for measuring physiological parameters (e.g., ECG), including a wearable support harness and a support structure for holding the electrodes in the garment against the skin.

FIGS. 18F and 18G show front and side views, respectively, of the support structure (expandable support structure) of FIG. 18A.

FIGS. 19A and 19B illustrate front and back views of pants.

FIG. 19C illustrates an exemplary connection between the garments disclosed herein.

FIG. 20 illustrates a wiring diagram for pants in accordance with some embodiments.

FIG. 21A illustrates a wiring diagram for the front of a garment in accordance with some embodiments.

FIG. 21B illustrates a wiring diagram for the back of a garment in accordance with some embodiments.

FIG. 22A illustrates another example of a garment as described herein, including sensors (not visible) and IMUs attached to the upper and lower legs, and upper and lower arms on both the right and left sides of the body.

FIGS. 22B and 22C show front and back views, respectively of another variation of a garment such as the one shown in FIG. 22A, having a IMUs arranged on the arms and legs, but also including EMG electrodes on the arm, legs and buttocks. Elastic fabric may be integrated into the compression fabric as shown, to further enhance the contact between the EMGs and the subject's skin. Five or more IMUs may be attached across the garment, including along the subject's back, corresponding to different spine regions, as shown in FIG. 22D. This may allow detection of posture for postural feedback, etc.

FIG. 23 is a front view of one variation of an apparatus as described herein, including ECG electrodes integrated into the garment, as well as respiration strain gauges and an inertial measurement unit (IMU) or other motion sensor(s). The sleeveless garment covers the torso, but integrates an adjustable elastic strip that may aid in keeping the sensors stably fixed to the skin. This variation may not include an expandable (e.g., inflatable) support, or it may include one or more expandable/inflatable supports.

FIG. 24 shows a back view of the apparatus of FIG. 23. The location of the ECG electrodes may correspond to locations for the traditional, 12-lead ECG positions when worn on a human body, as illustrated in FIG. 25.

FIG. 25 shows the target locations for traditional 12 lead ECG positions, showing positions for right and left arm/leg, (RA, LA, RL, LL) and the positions of the precordial leads (V1-V6).

FIG. 26A is a front view of an apparatus as described herein, including ECG electrodes integrated into the garment (additional sensors, such as respiration strain gauges and an inertial measurement unit (IMU) or other motion sensor(s) may also be included). The garment covers the torso and integrates an adjustable elastic strip that may aid in keeping the sensors stably fixed to the skin. Arm electrodes may also be secured against the skin (e.g., of the biceps) by one or more straps, as shown. In general, the location of the ECG electrodes may correspond to locations for the traditional, 12-lead ECG positions when worn on a human body, as illustrated in FIG. 25. The garment may include a fastener (e.g., zipper) on the side.

FIG. 26B shows a back view of the apparatus of FIG. 26A. A wireless communication device (e.g., phone, smartphone, etc.) may be held in an interface on the back; electrodes corresponding to leg electrodes may be included in the apparatus; alternatively connectors for arm/leg electrodes may be included.

FIG. 27A is a drawing showing a front side of an extended-wear monitoring garment.

FIG. 27B is a shows a back side of the extended-wear monitoring garment.

FIG. 28 illustrates an extended-wear monitoring garment.

FIGS. 29A and 29B show front and back views, respectively, of an apparatus as described herein, shown adapted for a female torso; the garment includes ECG electrodes integrated into the garment (additional sensors, such as respiration strain gauges and an inertial measurement unit (IMU) or other motion sensor(s) may also be included). The garment covers the torso and integrates an adjustable elastic strip that may aid in keeping the sensors stably fixed to the skin. Arm electrodes may also be secured against the skin (e.g., of the biceps) by one or more straps, as shown. In general, the location of the ECG electrodes may correspond to locations for the traditional, 12-lead ECG positions when worn on a human body, as illustrated in FIG. 25. The garment also includes adjustable straps that may help secure the sensors to/against the wearer

FIGS. 30A and 30B show front and back views, respectively of another variation of an apparatus, configured for a male torso, similar to that shown in FIGS. 29A-29B.

FIGS. 31A and 31B show front and back views, respectively, of a pair of pants (leggings, tights, etc.) that also include a plurality of sensors and may be worn independently or (as shown in FIGS. 31A and 31B) worn in conjunction with a shirt (such as the ones shown in FIGS. 29A-29B and 30A-30B.

FIG. 32A is an exploded view of one example of an electrode and expandable support (e.g., foam support) that may be included in any of the apparatuses described herein. This electrode may be configured, e.g., as an EEG, EOG, etc., electrode. FIG. 32B shows an assembled view of the embossed electrode without the cover, but including the support that may support the electrode securely against the skin, particularly in combination with the strap.

FIG. 33A is an example of a fabric cover for an electrode (e.g., an ECG electrode) and electrode support (e.g., expandable/compressible foam support) having a grip pattern. FIG. 33B is another example of a cover for a longer ECG electrode.

FIG. 34A-34F illustrates a breath sensor formed from a silicone conductive cord, and a method of making the breath sensor.

### DETAILED DESCRIPTION

In general, described herein are apparatuses (e.g., garments, including but not limited to shirts, pants, and the like) for detecting and monitoring physiological parameters, such as respiration, cardiac parameters, sleep, emotional state, and the like. In particular, described herein are stretchable, conductive sensors and connectors, which may include stretchable conductive inks, elastics, and traces that may be attached (e.g., sewn, glued, etc.) or in some variations printed onto garments, including in particular compression garments, to form sensors, conductive traces, and/or contacts.

U.S. patent application Ser. No. 14/023,830, titled “PHYSIOLOGICAL MONITORING GARMENTS,” and filed on Sep. 11, 2013 (incorporated by reference herein) describes exemplary garments any of which may be modified as described herein.

Any of the garments described herein may include one or more Sensor Manager System (SMS) placed directly onto the garment (e.g., shirt, shorts or in any other component of the wearable device, i.e. balaclava, socks, gloves, etc.), or integrated into the garment, as described in greater detail below. The SMS may include an electronic board. Connections to the SMS may be made by connectors including wire ribbon material (e.g., a stitched zig-zag connector) that may be included as part of the garment. In some variations a length of rigid material (e.g., Kapton) onto which conductive traces are attached, may be used.

An SMS that is integrated into the garment (as opposed to being provided by a separate device such as a smartphone) may provide numerous advantages. For example, an integrated SMS can manage a larger number of connections with the different sensors, and may processes the signals and communicates with the phone by means of a single mini-USB cable (e.g., independently of the number of signals processed). No matter the number of sensors that will be included in future devices (e.g., shirt, thighs, gloves, socks, balaclava, etc.), the connection between SMS and sensor module (e.g., phone) may always be based on a single 5-pin USB connection, thus substantially reduce the size of the female and male connectors from the device to the phone module. In a typical configuration, an SMS connects to a male connector through a UART (Universal Asynchronous Receiver-Transmitter) module and the male connector communicates to the mobile through another UART and an UART-to-USB module (see attached schematic and drawings).

An integrated SMS can be placed in different locations on the garment. For example, it may be placed at the base of the neck between shoulder blades, on the lumbar region on the thighs, on the arms, chest, or even on the socks, gloves, balaclava, etc.

An SMS may also be configured to communicate with different phones for the device. As mentioned, an integrated SMS may also allow you to have more connections (pins) to connect to different sensors/outputs. For example, an accelerometer may need 5 pins if you have the SMS present in a sensor module (e.g., mobile phone); an SMS integrated into the shirt may need fewer connectors, for example, such an SMS may need only 2 pins. With more sensors, without an integrated SMS the number of connectors may become unfeasible.

In general, the SMS may be a module (chip) that manages the signals from and to the sensors, and may act as an interface between the communication system (sensor module configured from a phone, etc.) and sensors. The SMS may manage the connection and interfaces between them. For example, and integrated SMS may include physical connections to sensors and may manage the way in which the signals are processed and sent between sensors and a sensor module and/or other analysis or control components. The SMS may also include or may connect to a multiplexer to alternate readings between various sensors to which it is connected.

In some variations, a SMS may provide proper power supply to passive sensors or active sensors. An SMS may take power from the mobile systems through a port such as a USB port. An integrated SMS may communicate from one side to a sensor module (e.g., communications systems/phone, etc. configured as a sensor module) through a USB port. The SMS may act as an interface or a bridge between the sensors and the sensor module.

In addition, any of the integrated SMSs described may be configured to include on-board processing (e.g., preprocessing), including, but not limited to: amplification, filtering, sampling (control of the sampling rate), and the like; typically basic pre-processing. An integrated SMS may also encode signals from the one or more sensors. In some variations the SMS may include a microcontroller on board. Further, and integrated SMS may also generally manage communication protocols to/from any or all of the sensors, and may make an analog to digital conversion (if the signals are analog) and may also communicate with a communications port of a USB, before going to the USB. For example, an SMS may be configured to convert the signal into UART to the USB signal protocol.

In addition or alternatively, any of the integrated SMSs may be configured as a signal receiver/transmitter. For example, an SMS integrated into the garment may be adapted to convert parallel signals to serial signals (in the order of the data).

As mentioned, an integrated SMS may be placed in any position on a garment, e.g., on or near the neck region, or more peripherally. Although the SMSs describe herein are referred to as “integrated” SMSs, these SMSs may be included on or in the garment (e.g., in a pocket or enclosure, though in some variations it is not physically connected/coupled to the fabric, but is instead placed on the garment. Thus, any of these SMSs may instead be referred to as dedicated or specific SMSs rather than (or in addition to) integrated SMSs. For example, the SMS may be placed under the female connector (housed inside the female connector), as part of the garment. When you wash the garment the SMS may get washed with the connector and the chip; the pins and SMS are waterproofed.

In some variations, the connectors (e.g., pins/ports) of the SMS are adapted to water resistant/water proof. For example, the pins used may make connections that are waterproof, e.g., with connections that only open when you engage the male pin, but are otherwise closed and waterproofed.

In any of these integrated SMSs, the SMS is a part of the garment, and are worn with the garment; the SMS module may pre-process the signal(s) to prepare them for transfer.

Thus, in any of the garments described, an SMS (Sensor Management System) may be included that is positioned on each garment (onboard/dedicated), rather than separate from the garment, e.g., as part of a separate sensor module, such as a general-purpose smartphone that may be held in a pocket on the garment, as previously described. Each garment may have an SMS (chip/microchip) that allows the garment to have connectors (female and male) with a numbers of pins (inputs/outputs) so that data from all the sensors in the garment (shirts, tights and accessories, such as gloves, socks, balaclava, etc.) may be first processed by the SMS and then sent through a connection (e.g., as few as 1 or 2 pins, or more) to the phone/communication module. In general, some of the sensors and components of the garments described herein may individually require multiple connections and thus a dedicated SMS may be very useful. For example, an IMU may require 5 pins and as many as 20 IMUs (or more) may be included as part of a garment, in addition to other sensors. Thus, the use of a dedicated SMS may allow the garment to manage a large number of data connections/contacts.

### Sensors

In addition to the sensors described in the Ser. No. 14/023,830 application included by reference in its entirety herein, such as touch point sensors, respiration sensors, bioelectrical sensors, etc., additional sensors may be included in any of the garments described herein. For example, a garment may include one or more skin conductance sensor. A sensor for measuring skin conductance can be made by two annular rings of the stretchable, conductive ink (see below) placed at the level of the third phalange of whatever couple of fingers (thumb, index, middle, ring and little finger). In some variations, the sleeve of the shirts has at the wrist level an integrated extension for this purpose. The skin conductance, depending on the sweating level, is measured as the inverse of the electrical resistance between the two considered ‘electrodes’ (annular rings).

Another integrated extension of the apparatuses described herein includes a full glove that, in addition or instead of a skin conductance sensor, incorporates a pulse-oximetry based on optical fibers. The use of optical fibers may also allow the incorporation other types of sensors. In addition, a full or partial glove may include additional sensors such as accelerometers, inertial measurement units (IMU), etc. Such glove-based sensors may allow applications in specific activities (e.g. playing a music instrument, type writing, etc.). A glove or pair of gloves may be configured to connect to other garments (e.g., shirts, etc.) or be formed as a sub-region of another garment (e.g., a shirt with finger regions/gloves, etc.).

Similarly to the gloves described above are socks or balaclava extensions, that incorporate other types of sensors, such as accelerometers, inertial measurement units (IMUs), EEG electrodes, etc. This allows applications in specific sports (e.g. football) and activities (e.g. playing chess).

### Production Processes

In general, the production of any of the garments described herein may include constructing the garment such as the sensors are held close and in stable contact with the skin. Thus, the sizing of the garment may be very precise, particularly in the following areas: thorax (because of different sizes of pectorals and breasts despite same corporeal size), abdomen (same reason), armpits, forearms, etc. The garments may be therefore precisely fit/manufactured, in addition to being made from compression materials. The design process may also include garment cutting.

Thereafter, any of the garments described herein may then be printed by, e.g., printing and transferring of the conductive ink traces and/or insulation. The printing may be performed by cylinder-type machines (because the printing is more precise and faster) using a heat transfer technique. For example, transfer on both sides of the fabric is performed at 150° C. for 15 seconds. Alternatively, garments may be printed by 3D printing, as discussed briefly below.

Thereafter, insulation may be applied (e.g., when capacitive touch points are used, such points may be insulated). The internal regions (i.e., in contact with the skin) of electrodes of a capacitive touch point may be insulated by heat-welding a layer of high quality polyurethane film exactly reproducing the shape of the electrodes. The size of the insulation layer may be slightly larger than the size of the electrode to allow a complete covering thus to avoid ‘lateral’ contamination of biopotentials.

In variations in which higher conductive connections are used, the apparatus may include the addition of higher-conductive substrates and materials, such as wire ribbon material (e.g., stitched zig-zag connectors) as described herein. Thus, the formation process may then include the application of these wire ribbon material connectors, which may include connecting the ends of the wires (forming the wire ribbon material) to the sensor(s) and/or SMS components. The wire ribbon material may include a substrate of compression fabric that may be fused, glued, stitched, or otherwise connected to the body of the garment. For example, once positioned, the wire ribbon material (e.g., a stitched zig-zag connector) may be secured to the fabric through high quality polyurethane tapes for heat-welded applications. In some variations, rather than (or in addition to) the wire ribbon materials, a more rigid or semi-rigid substrate may be used, such as Kapton, onto which electrical traces, and/or circuitry, may be printed. In order to maximize comfort of movement, the electronics on the Kapton may be designed to have a single layer, thus minimizing its thickness.

The garment may then be sewed. The sewing may be performed by traditional processes, although in some variations, sewing over conductive ink, the wire ribbon material, or Kapton traces may be avoided.

At the same time or thereafter, soldering may be performed, e.g., to connect the wire ribbon materials, and/or regions including an additional (e.g., Kapton) substrate for higher-conductive traces, with printed conductive ink sensors, electrodes and/or traces. For example, soldering between ink traces and Kapton terminals may be performed by using conductive epoxy, successively covered by a high quality polyurethane film.

Thereafter, in some variations a semi-rigid collar region may be attached, e.g., to secure and cover an integrated SMS module and connectors. A collar may be made of a polyurethane material that takes the shape of the user's shoulders and may be applied by thermal welding through a transfer machine with plates custom-made to fit the body surface in the neck region.

In some variations, the method of forming the garments may also include the addition of ‘stretching limiters’ made, e.g., of stripes of polyurethane material with limited elongation. They may be positioned by thermal welding in the inner part of the garment, in proximity of long ink traces (e.g. respiration traces), in order to prevent overstretching (e.g. during wearing) that could either break a trace, or determine permanent elongation, that must be avoided for functional and aesthetic reasons. To enhance their strength, they may be positioned in a way to run between two seams

In some variations the garment may be produced by installing a silicone cord. To avoid stretching of the garment and its sensors when the user is wearing the garment and putting the garment on, a cord made of silicon may be applied (e.g., by thermal welding) to the lower edge of the garment, running all around the edge. This may allow the wearer to easily pull the shirt down from the armpits to the waist after the collar and the sleeves have been inserted, without overstretching the garment.

As mentioned above, the garment described herein may be made entirely or in part by a 3D printing technique. For example, sensors and/or conductive traces and/or connectors may be produced by 3D printing. In some variations a fabric (e.g., compression garment fabric) may act as a substrate for the 3D printing. In some variations the fabric may itself be created or modified by 3D printing. Thus, a garment may be made by transfer and direct printing (3D printing). In, one example, a 3D printer for producing a garment including the integrated sensors described such as those described herein may include at least three nozzles: one nozzle may be adapted to print a compression garment fabric; one nozzle may be adapted to print/insert a stretchable conductive ink; and one nozzle may be adapted to print/insert sensors and/or electronics. In contrast with currently practiced methods, which may require weaving the fabric (e.g., from thread), printing the electronics and sensor on the fabric (or onto a substrate and then transferring to the fabric), then sewing the fabric, in 3D manufacturing, production can go directly to printing threads, ink and electronics based on precise personal measurements from a person, which may be both more accurate and faster.

### Materials

In general, the garments described herein may include a compression fabric to secure that sensor are in good permanent contact with the skin. For example, the anterior part of the shirt may have a lower percentage of elastane (between 5 and 20%) than the rest of the body, which may include a higher percentage of elastane (between 15 and 40%). The fabric may be stretchable into two ways (one direction) and may be positioned with the least stretchable side placed horizontally to respect human body which dynamically stretches more horizontally than vertically. In general, a compression fabric may be any fabric having the material properties associated with compression fabrics as described herein.

Examples including materials such as fabrics made of elastic polyurethane fibers (e.g., elastin fibers, Lycra, etc.).

As discussed in greater detail below, any of these garments may include a stretchable conductive ink and/or a stretchable insulator (over/surrounding) the conductive ink. Both the conductive ink and the insulator may be stretchable, up to some percentage, X % stretchable (e.g., up to 5% stretchable, up to 6% stretchable, up to 7% stretchable, up to 8% stretchable, up to 9% stretchable, up to 10% stretchable up to 11% stretchable, up to 12% stretchable, up to 13% stretchable, up to 14% stretchable, up to 15% stretchable, up to 16% stretchable, up to 17% stretchable, up to 18% stretchable, up to 19% stretchable, up to 20% stretchable, up to 21% stretchable, up to 22% stretchable, up to 23% stretchable, up to 24% stretchable, up to 25% stretchable, up to 30% stretchable, up to 35% stretchable, up to 40% stretchable, up to 45% stretchable, up to 50% stretchable, etc.). This may also be expressed as more than X % stretchable (e.g., more than 5% stretchable, more than 6% stretchable, more than 7% stretchable, more than 8% stretchable, more than 9% stretchable, more than 10% stretchable more than 11% stretchable, more than 12% stretchable, more than 13% stretchable, more than 14% stretchable, more than 15% stretchable, more than 16% stretchable, more than 17% stretchable, more than 18% stretchable, more than 19% stretchable, more than 20% stretchable, more than 21% stretchable, more than 22% stretchable, more than 23% stretchable, more than 24% stretchable, more than 25% stretchable, more than 30% stretchable, more than 35% stretchable, more than 40% stretchable, more than 45% stretchable, more than 50% stretchable, etc.). Stretchable typically mean capable of being stretched (e.g., by applying a force such as a pulling force) from a starting length/shape and returning to approximately the starting length/shape. In some variations may mean additionally or alternatively, resisting breaking when a deforming force (elongating or distorting from the original length/shape) is applied (and eventually released). Examples of stretchable conductive inks and characteristics of such inks are provided below.

As mentioned, any of the garments may also include a substrate attached or formed as part of the garment for higher-conductive paths, such as Kapton films. Other flexible, wearable substrates may also be included. Any of the garments may also include one or more polyurethane films and tapes for sewn and heat-welded applications (e.g., high-quality polyurethane films and tapes). In addition any of the garments may also include an electrical insulation material (e.g., polyimide materials, etc.) for covering/insulating a conductive trace, forming a part of a sensor, or the like.

A substrate such as Kapton may be fixed to on onto the garment. For example, the substrate may be sewn and/or attached by an adhesive, etc. The substrate may be held in a pocket or other region of the garment. As mentioned above, any of the garments may include a limiter (e.g., stretch limiter) of a second material (e.g., a cloth material that is less stretchable than a compression garment, etc.).

Any of these garments may also or additionally include silicone for sewn and heat-welded applications.

Strechable Conductive Inks

In general, the stretchable and/or flexible conductive inks products described herein may be formed of an adhesive (e.g., glue, such as acrylic, polyamide and other adhesives) onto which a printable mixture of conductive solution is applied. The wet-applied conductive solution (which may be referred to for convenience as the conductive ink, even though the final conductive ink product includes the adhesive material layer) is typically applied as a layer onto the layer of adhesive, so that an intermediate region between the adhesive and the wet-applied conductive solution forms. This intermediate region may be important for the conductive and stretchable properties of the resulting conductive ink material. The intermediate region is a gradient region, because it defines the concentration gradients of the adhesive layer and the wet-applied conductive solution (conductive ink). This is illustrated and described below.

A stretchable, conductive ink (the we-applied conductive ink layered against the adhesive) typically includes a percentage of conductive material (e.g., around/approximately 20%, 25%, 30%, 35%, 40%, 45%, 50%, 55%, 60%, 65%, 70%), and a biocompatible binder (e.g., acrylic binder that is formaldehyde-free such as water-based acrylic binders, water-based polyurethanes, etc.), a thickener (e.g., polyurethanic thickener) and an optional humectant and/or solvent (e.g., propylene glycol). The stretchable conductive inks as described herein generally meet a minimum conductance as well as a minimum stretching property. The stretchable conductive ink may also optionally include a de-foamer to eliminate air/foam when processing (e.g., 1-butanol), a catalyst (e.g., to aid in crosslinking of the binder, eg, amine compounds or metal complexes), and additional additives which may help with the printability and stability of the product.

In one example, a stretchable conductive ink (and particularly the wet-applied conductive ink portion) is formed of: 50% Carbon Black, 40% Acryilic Binder, totally formaldehyde-free, 5% propylene glycol, and 5% polyurethanic thickener. The conductive material (Carbon Black) may be particulate. Carbon Black may be preferred, particularly compared to other conductive materials such as silver or other metallic. Other conductive materials may include graphene, graphite, coated mica (e.g., mica coated with an oxide, such as antimony-doped tin dioxide, etc.), or the like.

The conductive inks described herein are not only conductive, but also stretchable and therefore can work properly on compression garments. In addition, the stretchable conductive inks appropriate for forming the garments described herein may be ecologically appropriate (e.g., having a formaldehyde concentration lower than 100 ppm), and resistant to washing (with preservation of electrical and elastic properties after multiple washes).

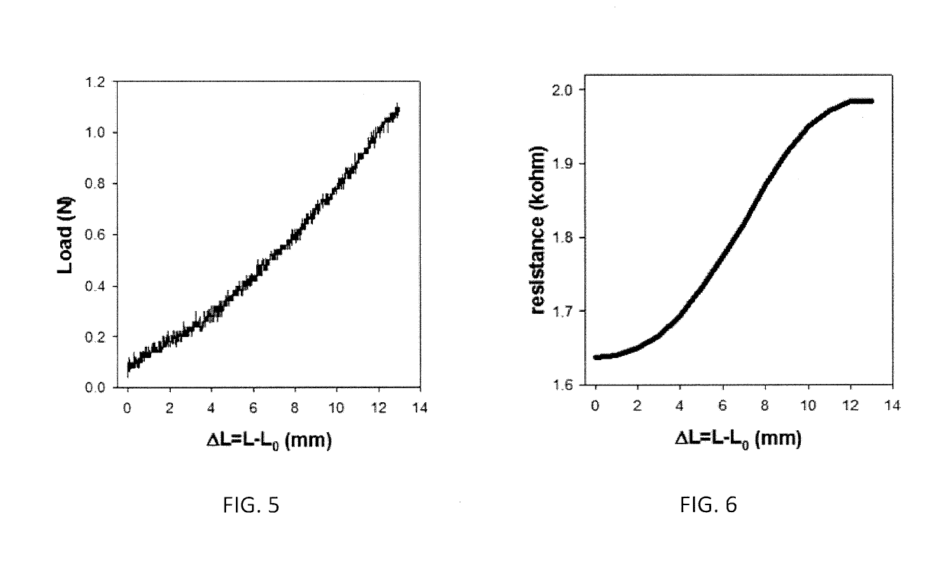

Experimental studies have confirmed that the stretchable conductive ink compositions (layered structures including the intermediate, gradient region between the adhesive and the wet-applied conductive ink) described herein are stretchable. FIGS. 5 and 6 illustrate preliminary results of testing conducted on a sample of conductive ink printed on a compression textile as described above. A video camera was used to demonstrate that no fractures developed in the ink during the extension (e.g., change in length of up to 13 mm was examined). The conductance (e.g., resistance) varied with applied force between approximately 1.6 kOhms to 2 kOhms, while a linear stretch was observed up to 1.1 N (e.g., stretch up to approximately 13 mm without breakage at approximately 1.1N). In general, the stretchable conductive inks described herein may be within a performance range of being stretchable up to at least 1 N of force (e.g., up to at least 2 N, up to at least 3 N, up to at least 4 N, up to at least 5 N, up to at least 6 N, etc.) and/or stretchable (without breaking) up to at least 5 mm (e.g., up to at least 6 mm, up to at least 7 mm, up to at least 8 mm, up to at least 9 mm, up to at least 10 mm, up to at least 11 mm, etc.) and/or stretchable up to a ratio of applied stretching force (in N) to extension length (in mm), e.g., around about 1 N/mm without breaking. Surprisingly, in the experiment shown in FIG. 5, the conductive traces examined did not evidence any breakage up to almost 2 N, which is a reasonable near-maximal force that may be applied when applying/wearing a garment. Neither macro (visible to the naked eye) nor micro breakage was apparent.

In general, the resistance of the stretchable conducive ink may depend upon the size of the trace, including thickness, length, etc. (which may vary under stretch) and may be lower than about 5 kOhm (e.g., less than about 4 kOhm, less than about 3 kOhm, less than about 2 kOhm, etc.) at rest and under a predetermined stretch force (or force/stretch length). In general, the resistance may be within a range of a few hundred Ohms to a few hundred kOhms. In FIGS. 5 and 6, the tested stretchable conductive ink was printed on the compression garment fabric to a length of 60 mm and a width of about 10 mm; eight layers of ink were applied to form the final thickness (which was less than about 2 mm (e.g., approx. 1 mm or less).

Stretchable conductive inks that may be used to forms trace, connectors and/or sensor in any of these garments described herein are described in greater detail below.

### Systems

Any of the garments described herein may be used as part of a system including multiple garments that connect (either or both directly connect or wirelessly connect). For example, and upper body garment/device may connect with lower body garment/device. Signals from sensors positioned on garments on the lower part of the body (e.g., shorts, thighs, socks, etc.) may be transmitted to one or more SMS, e.g., on an upper garment such as a shirt, etc. A connection may be made through a support substrate (e.g., Kapton) including traces that can connect through a connector positioned in an internal portion of the upper garment (e.g., the lower hem region of the upper garment).

In general, the garments described herein may include a body formed of a fabric. In particular, compression fabric materials are useful. The body may include a plurality of sensors positioned in predetermined locations on the garment. The sensors may be on the inside of the garment (e.g., facing the wearer), or they may be on the outside of the garment. Connectors may connect the sensors to one or more sensor manager/sensor module (SMS) that may include a processor. The SMS may either directly transmit or connect/couple to a sensor manager unit (SMU) for recording/analyzing/transmitting the sensed data, or it may itself perform some or all of these functions. In general, the sensors may be formed at least in part of the stretchable conductive ink structures described herein (e.g., as used herein, “stretchable conductive ink” structures may refers to the combination of the wet-applied conductive ink, adhesive and gradient/intermediate region between them described herein). A sensors including the stretchable conductive ink may include a touch point (e.g., capacitive) sensor, a skin electrode sensor, or the like. Also described herein are sensors formed at least in part of conductive elastic ribbon (e.g., elastic saturated with conductive particles in a base/binder, as described herein), which may form strain gauges or other sensors. The connectors may be formed of stretchable conductive ink and/or conductive elastic ribbon. In some variations the connector is formed of a wire ribbon material (e.g., stitched zig-zag connector) in which enameled wires are sewn onto strips of material (e.g., compression fabric) in a sinusoidal/zig-zag pattern, and the ribbon is applied to the body of the garment. In some variations the connector may be a rigid or semi-rigid substrate (such as Kapton) onto which electric traces and/or circuitry are applied; the substrate may be attached and/or covered in fabric such the compression fabric and attached to the body of the garment, or directly attached to the body of the garment.

Any type of garment may be formed as described. For example, described herein are garments configured as medical devices, or for use a medical device, including a monitoring device, therapeutic device, or aid. The body of these garments may be formed of a compression fabric (entirely or in part), and the garment may be fit to the body, to help adhere the sensor(s) against the subject's body securely. In some variations the garment (e.g., medical device) may include additional elements, such as straps, halters, bra, yoke, harness, etc., or the like to help secure a portion of the garment against the subject's body. In some variations the garment may include an expandable (e.g., inflatable) support structure on a portion of the garment to help hold or secure a sensor (or sensors) against the subject. An expandable support structure may be used with a harness. The harness may be separate, or it may be integrated into the garment.

For example, described herein are garments configured to sense electrocardiographic (ECG) signals for recording and/or analysis. Such garments may be configured to connect to the wearer's (subject's) upper body, and may be in the form of a shirt or may include a torso covering. These garments may include at 5 or more electrodes, e.g., six chest electrodes and three or more electrodes for each of the right arm, left arm and a leg. Additional electrodes may be used. In some variations, the chest electrodes are pairs of electrodes that may be redundant.

Any of these garments may also or alternatively include one or more respiration sensor(s). In general, these respiration sensors include a fabric and/or conductive ink-based strain gauge. For example, the strain gauge may be formed of the stretchable conducive inks described herein and/or the conductive elastic strips described herein. In one variation, the garment includes 10 ECG sensing electrodes, 2 respiration sensors (strain gauges). The ECG electrodes may be located on the chest of the garment so that they contact the skin of the user in the position where standard 12 leads would be placed. The respiration sensors may be positioned on garment so that the compression garment, when worn, holds them against the body near the Xyphoid and Umbilicus height on the subject's torso. The sensors may be connected to SMS units by traces, such as an elastic strip with a copper-wire ribbon and/or a stitched zig-zag connector. The garment may include both a shirt (and some variations, tights).

Also described herein are garments configured to measure respiration (including regional respiration). For example, a garment may be configured to include a shirt portion formed of compression fabric that detects respiration (e.g., to allow plethysmography of the sensed signals). The apparatus may also include electrodes as described above to detect a simple ECG signal (e.g., having 2 electrodes, or a single lead, or multiple leads, e.g., 3 leads, 5 leads, 12 leads). For example, 12 respiration sensors (e.g., conductive elastic strip strain gauges as described herein) may be included. The respiration sensors may be located for positioning on the wearer near the Louis angle, 3rd costal interspace, xyphoid, lower costal margin, above the umbilicus, and below the umbilicus. There may be duplicate (e.g., left side/right side of the wearer's trunk) sensors. The sensors may be connected to one or more SMS units via a connector such as a stitched zig-zag connector (e.g., in which a strip or tube or compression garment fabric is stitched in a sinusoidal pattern with an insulated/enameled copper wire).

Garments as described herein may also be configured a garments to sense sleep disorders, and may include a head covering portion as well as a torso and/or pant portion. Such garments may include, e.g., EEG electrodes (e.g., one or more) and thus ECG electrodes, respiration sensors, and one or more Inertial Mass Unit (IMU) to detect activity level and basic movements. For example, a garment may include 21 EEG electrodes (formed of stretchable conductive ink, or alternatively standard medical electrodes may be used), two ECG electrodes (formed of stretchable conductive ink), and 2 respiration sensors (formed of conductive elastic strips), and five IMUs. The EEG electrodes may be positioned as a simplified 10-20 system on a head covering, while the ECG electrodes may be positioned on the right and left trunk portion of the garment. Respiration sensors may be positioned so that they are worn near the xyphoid and umbilicus. The IMUs may be positioned on the lower back and limbs (e.g., arms on the shirt, legs on the tights)

Garments for use as a fitness tool or aid are also described herein. For example, described herein are garments configured as a fitness device may include sensors for detecting body status and athletic performance. These garments may monitor body status (e.g., well-being) by sensing and/or measuring indicators of heart rate, respiration, body fat, movement, posture, and stress-level. For example one variation of a fitness garment may have a body formed of a compression fabric with two ECG sensors (electrodes, e.g., formed of stretchable conductive ink), one respiration sensor (e.g., formed of a conductive elastic strip), and four IMUs. The ECG electrodes may be positioned in the garment to be held against the right and left trunk regions, the respiration sensor may located on the garment to be held against the xyphoid region, and the IMUs may be positioned on the lower back, one in each forearm, and one in an SMS unit (e.g., near neck region). In some variations the apparatus may also include body fat sensors at the wrists, neck and umbilicus region. A body fat sensor may be an electrode (e.g. formed of a stretchable conductive ink).

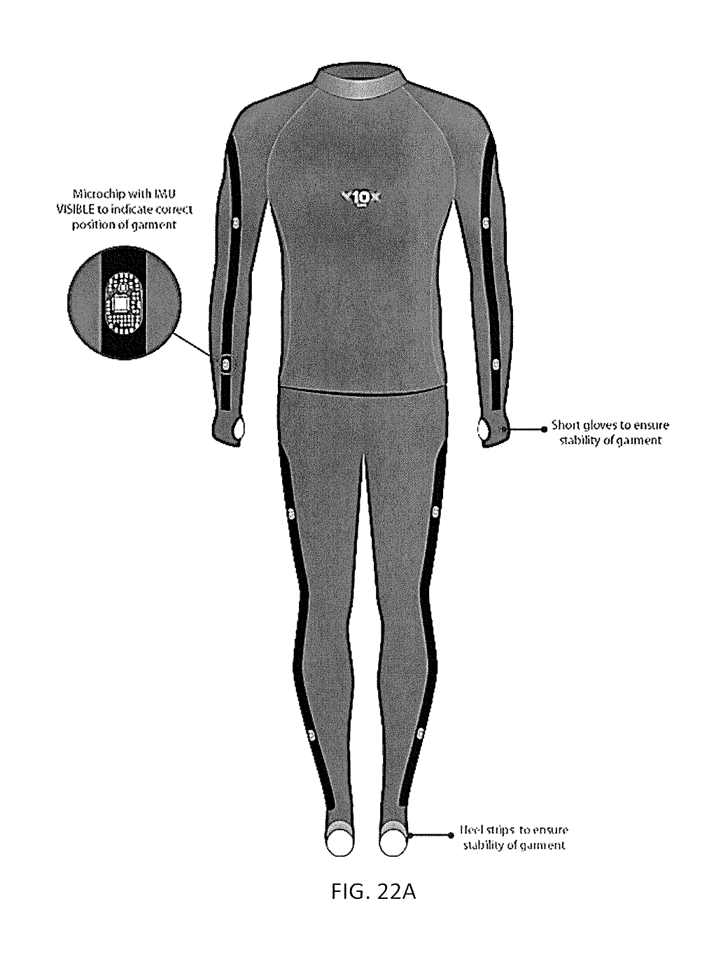

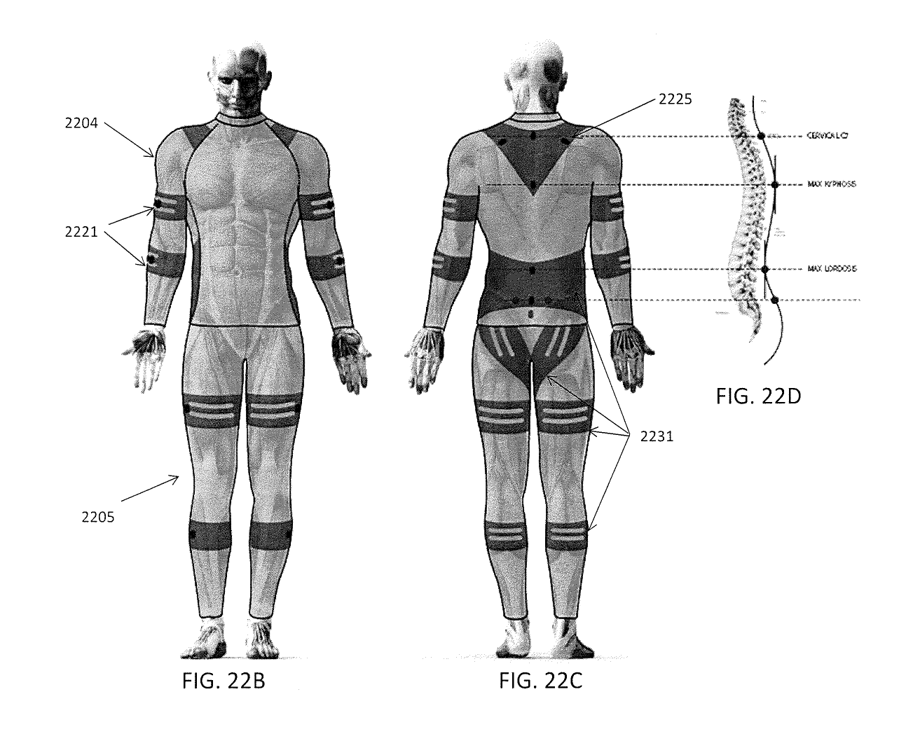

Another variation of a fitness garment (e.g., general fitness garment) may be configured as a shirt to be worn on the upper body. As mentioned, these garments may be used to monitor general well-being, and may operate with a controller that compares data for references as well as evaluating basic fitness skills such as coordination, equilibrium, stamina, ‘breath’, strength, flexibility and reflexes. In some variations, the garments include at least three (e.g., 4) IMUs in the upper portion (the shirt, e.g., upper and lower arm, left/right) and at least three (e.g., 4) IMUS in the lower portion (e.g., pants/tights, upper and lower leg, left/right), a respiration sensor (in a region to be worn against the umbilicus region). See, e.g., FIG. 22A-22D. In the example shown in FIG. 22B, the system has two parts; a shirt for detecting posture and monitoring fitness; and a pair of pants that can connect to the shirt or separately connect to a processor. In FIG. 22B, the shirt 2204 and pants 2205 including EMG sensors 2221, shown as parallel lines of sensors. IMUs 2225 are also positioned at the upper and lower legs, upper and lower arms, and along the back, so as to detect postural changes. Bands of elastic material 2231 are integrated into the compression further help hold the electrodes (e.g., EMG 2221) against the skin, as shown by the darker regions.

In general, any of the garments may operate with/connect to a processor that can store, transmit, compress, and/or analyze the recorded data.

Examples of these various types of garments are described below.

### Garments That Detect Respiration

Garments may be adapted to detect respiration, and in particular, regional respiration. Such devices may be used at the request of a medical professional, or by anyone who wishes to monitor respiration. A respiration-monitoring device may be adapted for the continuous and accurate monitoring of respiration, including monitoring of respiration in one or more regions. A complete and accurate measurement of several respiratory parameters (described below) may be made using a plurality of stretchable conductive ink traces arranged in a pattern (e.g., a ‘zig-zag’ pattern) arranged in different region of the garment so that they are positioned about a wearer's torso; alternatively in some variations a conductive elastic strip (e.g., an elastic strip that has been impregnated with a conductive material) may be used in addition to or in place of stretchable conductive ink traces. Regions including lengths of stretchable conductive respiration sensors may include: the anterior part of a shirt, the posterior part (back) of a shirt; each or either of the two lateral sides of a shirt, etc. Sub-regions within these regions may also be used. The stretchable conductive respiration sensors, as described above, may have a resistance that varies slightly with stretch; this property may be used to detect and/or measure body movement as the sensor is stretched while worn on the body.

As described below, in some variations, four or more respiratory signals may be measured to determine localized respiration. For example, twelve signal may be measured by grouping the variable resistances of the traces (or an average of numerous traces) that are placed in the following areas/regions: (1) anterior, upper right (e.g., 6 traces); (2) anterior, upper left (e.g., 6 traces); (3) anterior, lower right (e.g., 5 traces); (4) anterior, lower left (e.g., 5 traces); (5) posterior, upper right (e.g., 6 traces); (6) posterior, upper left (e.g., 6 traces); (7) posterior, lower right (e.g., 5 traces); (8) posterior, lower left (e.g., 5 traces); (9) lateral, upper right (e.g., 3 traces); (10) lateral, lower right (e.g., 5 traces); (11) lateral, upper left (e.g., 3 traces); (12) lateral, lower left (e.g., 5 traces). Based on the arrangement of stretch-sensitive conductive traces and/or elastic strips, parameters may be extracted by analysis of the different signals. For example, a measure of total tidal volume may be determined by adding the signals from all of the stretch sensors in each region (e.g., 1+2+3+4+5+6+7+8+9+10+11+12). A measure of rib cage tidal volume may be determined by adding the signals from the upper regions (e.g., 1+2+5+6+9+11). A measure of abdominal tidal volume may be determined by adding the signals from the lower (abdominal) regions (e.g., 3+4+7+8+10+12). A measure of the rib cage respiratory region may be determined by adding just the region associated with the right rib cage (e.g., 1+5+9); a measure of the left rib cage may be measured by adding just the regions associated with the left rib cage, (e.g., 2+6+11). A measure of the respiration in/at the right abdominal region may be determined by adding the signals from the right abdominal region (e.g., 3+7+10), and similarly a measure of the respiration in/at the left abdominal region may be determined by adding the signals from the left abdominal region (e.g., 4+8+12).

From the time course of the signals (e.g., the signal of the total tidal volume), temporal parameters of breathing, such as respiratory frequency (f), inspiratory time (Ti), expiratory time (Te), and/or duty cycle [Ti/(Ti+Te)] can be determined, recorded, measured and/or displayed (as can any of the signals detected on the garment).

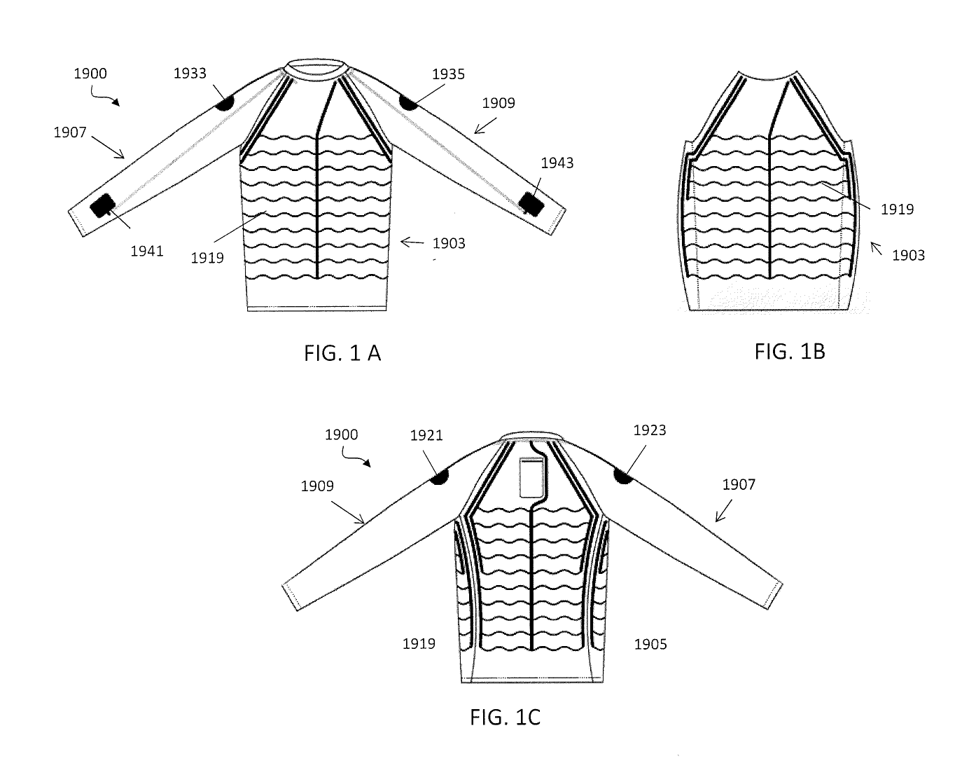

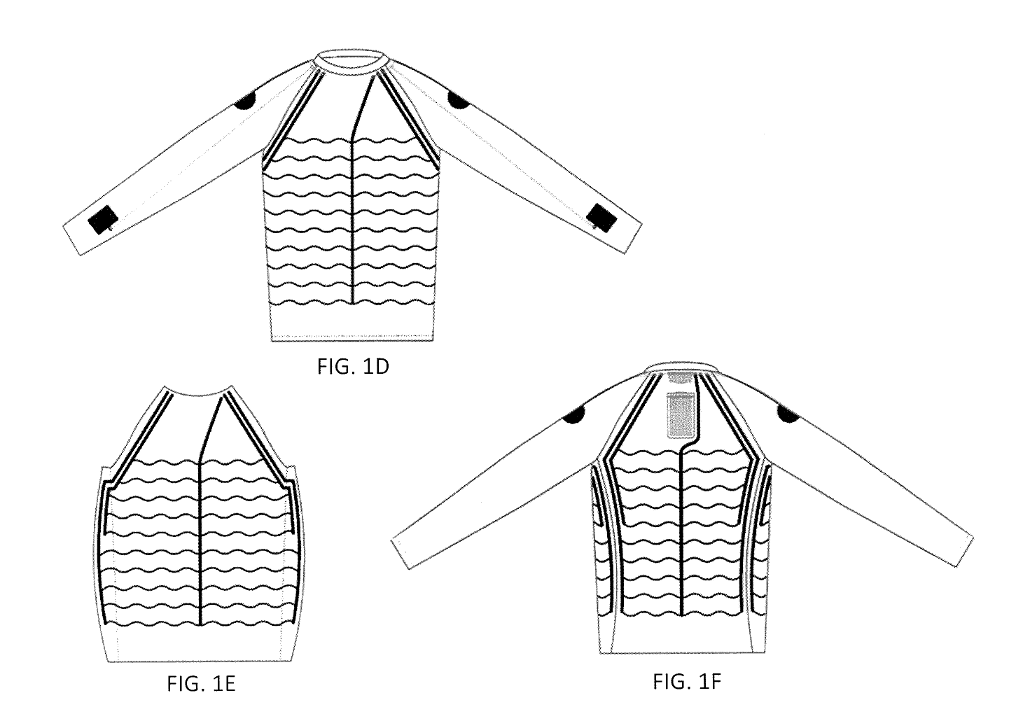

For example, FIGS. 1A-1C illustrate one variation of a shirt for detecting and/or monitoring, including continuous monitoring, respiration. In any of these examples, the apparatus, which may be referred to interchangeably as a device or system, may be configure to continuously and accurately monitor respiration The shirt shown in FIGS. 1A-3A are compression garments (shirts) typically composed by four parts: (a1) 1903 anterior and lateral sides; (a2) 1905 posterior (back); (a3) 1907 right arm; (a4) 1909 left arm. These parts are sewn together after deposition of conductive ink, conductive connector (e.g., Kapton with conductive material and/or wires stitched in a zig-zag pattern) and layers of insulating material, e.g., by a transfer process.

In general, conductive ink traces may be used as sensor. In FIGS. 1A and 1B, the sensor is a plurality of conductive ink traces that are stretchable traces. Conductive ink (including the conductive ink, adhesive and gradient region) may be used to form the conductive traces 1919, as described herein. Any of these devices may also include a sensor manager unit. The sensor management unit 1921 may be a processor that is placed on the garment (e.g., on the back) in connection with an interface for connecting the sensors to the processor. The processor may be, for example, a smartphone or other handheld device. The apparatus may have a communication unit; this communication unit may be separate or may be integrated with the processor (and/or may include its own dedicated processor). For example, a communication unit may also be placed on the back, and connect to the interface.

Additional sensors may also be used, including motion sensors. For example, a tri-axes accelerometer (alone or, e.g., embedded in the communication system), may be included.

In general, any of these devices may include one or more wearer inputs, such as ‘touchpoint sensors’. For example, two capacitive touch points 1933, 1935, placed on the arms, may be used. A touchpoint sensor may include two electrodes (e.g., one on the inner, the other on the outer, surface of the garment in corresponding positions), made of conductive ink patterns, a separating layer of the textile between the two conductive electrode patterns; and an insulating layer deposited onto the internal conductive ink pattern layer. A connecting trace may be included between the external electrode and a terminal point placed close to the neck.

Additional sensors may include one or more electrodes, such as an electrode to detect hear rate. For example, two electrodes 1941, 1943 for heart rate (HR) measurements, made of conductive ink, may be placed on the inner surface of the right and left arms of the shirt. These electrodes may be connected by a conductive connector such as a conductive (Kapton) traces connecting the HR electrodes to the terminal point close to the neck, as shown in FIG. 1A and 1C.

In general, the respiratory traces may be positioned in any region of the body of the shirt to detect movement (expansion/retraction) due to respiration in that portion of the body. A complete and accurate measurement of several respiratory parameters (see below) may be provided for individual regions of the wearer's body by positioning conductive ink stretchable traces, ‘zig-zag’ shaped, (e.g., by transfer process) in different regions of the body of the shirt. For example, conductive traces and/or conductive elastic strips may be positioned on the anterior and the two lateral sides of the shirt, on the posterior part (back) of the shirt, and in various sub-regions of these portions.

In FIGS. 1A-1C, eight signals are measured by the sensor manager unit (processor) as voltage variations determined measuring by the variable electrical resistance of the traces placed in parallel in the following areas:

1. Anterior+lateral, upper right (5 traces in parallel).

2. Anterior+lateral, upper left (5 traces in parallel).

3. Anterior+lateral, lower right (5 traces in parallel).

4. Anterior+lateral, lower left (5 traces in parallel).

5. Posterior, upper right (6 traces in parallel).

6. Posterior, upper left (6 traces in parallel).

7. Posterior, lower right (5 traces in parallel).

8. Posterior, lower left (5 traces in parallel).

The vertical traces shown are made of conductive ink and/or conductive elastic strips, and constitute the terminals of the total electrical resistance in these 8 areas. These respiration sensors (respiratory sensors) are connected to terminal points positioned close to the neck (at the interface region). A processor or other circuitry may be used to detect/monitor resistance. For example, in some variations a sensor manager (processor) may be used to obtain and/or store, transmit, analyze, process, etc. the 8 signals listed above. The processor may also incorporate and analyze, transmit, process and/or store additional signals, including the signals obtained by summing one or more combination of single signals. For example, as mentioned above:

Total=1+2+3+4+5+6+7+8

Rib cage signal=1+2+5+6

Abdominal signal=3+4+7+8

Right rib cage signal=1+5

Left rib cage signal=2+6

Right abdomen signal=3+7

Left abdomen signal=4+8

From the time course of the signal of total signal, the following temporal parameters of breathing can be obtained: respiratory frequency (f), inspiratory time (Ti), expiratory time (Te), duty cycle [Ti/(Ti+Te)], etc.

As mentioned above, these signals may be stored, transmitted, analyzed, etc. by the processor and/or communications unit.

FIGS. 1D, 1E and 1F show another compression garment including regional respiratory sensors similar to the garment shown in FIGS. 1A-1C.

As mentioned above, in some variations a respiration/respiratory sensor include a breathing sensor that is a conducive elastic strip that has be treated to have a resistance that varies with stretch, with a relatively small (or negligible) mechanical and very low electrical hysteresis in cyclic loading. Such sensors may be referred to herein as conductive elastic strip sensors, or conductive elastic strain gauges. Described herein are conductive elastic materials, and method of making and using them. In particular, described herein are methods of forming conductive elastic materials that may be used as part of a sensor (e.g., stretch or respiratory sensor) on a wearable garment, including in particular wearable stretch (e.g., compression fabric) garments. The conductive elastic materials described herein may be used, for example, in any of the garments including respiration or other contact and/or stretch sensors.

The conductive elastic materials described herein may change resistance as they are stretched, and therefor act as a stretch sensor. Further, these materials may have superior mechanical and electrical properties when compared to other stretchable conductive materials, as they have a very high mechanical and electrical memory. This means that they may be stretched, e.g., to as much as 1.2× (or in some variations: 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 3×, 3.1×, etc.) the original, rest, length and return to the same resting length. The dimension of stretching (length, width, etc.) may be the same. In some variations the material maybe more stretchable in one dimension (e.g., length) than the other (e.g., width). Below this upper limit of stretch (e.g., 1.3× the original length, or in some variations: 1.5×, 1.6×, 1.7×, 1.8×, 1.9×, 2×, 2.1×, 2.2×, 2.3×, 2.4×, 2.5×, 2.6×, 2.7×, 2.8×, 3×, 3.1×, etc.) the material does not exhibit substantial hysteresis, and will return to the original resting length.

Similarly, the material may experience little, if any electrical hysteresis with stretch below a relatively high limit of stretch. For example, the material may have approximately the same conductance/resistance after being stretched up to 1.2× (or in some variations: 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 3×, 3.1×, etc.) their original, rest, length. Further, the change in resistance with stretch may linear over at least a portion of the range. Thus, the materials described herein exhibit very little electrical hysteresis with use. Further, these properties may be repeatable for a long period of time (e.g., over many hundreds, thousands or hundreds of thousands or cycles of stretch.

Finally, the response time in recovering from stretch may be extremely fast. For example, the material may return to the initial performance measurements (for length and resistance) within a less than 5 seconds (e.g., less than 4 seconds, less than 3 seconds, less than 2 second, less than 1 second, etc.). Thus, the electric return time is faster than 5 seconds over the entire stretch range (of less than the maximum stretch length, e.g., 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 3×, 3.1× the original length).

These properties, and particularly the electrical properties, of the material appear to result from a reduced molecular damage of the conductive material during even repeated stretch cycling. This may result in an increase in the length of the lifetime of the material. Further, the elastic properties (the return from stretch) appear to be drive by the core elastic material to which the conductive material (e.g., conductive coating) is coated. The core material appears to keep is elastic properties even when coated with a relatively thick coating of (dried) conductor.

A stretch sensor may be made of an elastic material. For example a conductive elastic material may be made by first coating (e.g., dipping, spraying, submerging, etc.) an elastic material, and particularly an absorbent or partially absorbent elastic material, into a suspension of electrically conductive particles in a solution. Any appropriate conductive material may be used, including, but not limited to carbon black, metallic conductive materials (e.g., gold, silver, silver/silver chloride, graphene, mica coated with oxide, etc.). The conductive material may be a mixture of conductive particles suspended in a solution (such as water or alcohol solutions). The solution may include a base or binder as well as the solution of conductive particles. For example, the solution may be between 0.1-25% binder (e.g., acrylic, water based polyurethane, etc.) and 99.9%-75% solution of conductive particles. Once applied to the conductive wires, the elastic ribbon may be enclosed within a fabric (e.g., an insulating fabric, which may be the same as the fabric to which it's being applied). In some variations the elastic ribbon may be enclosed in an insulator material and/or coated with an insulator. The tissue (covering) ribbon may be fixed over the elastic ribbon by, e.g., thermo press (when using a thermally activated adhesive). Thereafter, the resulting ribbon including the conductive elastic material and zig-zag wires may be attached to a garment, such as a compression garment.

Thus, the conductive elastics strips described above may be used as part of a compression garment. Described above are method of making and using conductive elastic material having very low mechanical and electrical hysteresis and may therefore be used as respiration sensors for wearable compression garments. This conductive elastic material may be used as a respiration/respiratory sensor, or as part of a connector. A respiratory sensor using a conductive elastic material may be formed of a strip of elastic material that has been impregnated with a solution of conductive particles (e.g., carbon black) and dried (or at least partially dried); conductive connectors may be attached to the ends of the strip of impregnated elastic material. In some variations the connector may be electrically connected to a wire ribbon material formed of enameled (e.g., insulated) metallic conductive wires that are stitched in a zig-zag pattern on a strip of fabric, such as compression fabric. The conductive-particle impregnated elastic material and/or the wire ribbon connector material may be enclosed within a fabric material (such as a compression fabric material). This enclosed sensor and connecting length(s) of wire ribbon may then be attached to a garment as described above (e.g., see FIGS. 1A-1F).

### Electrocardiogram (ECG) Measuring Garments

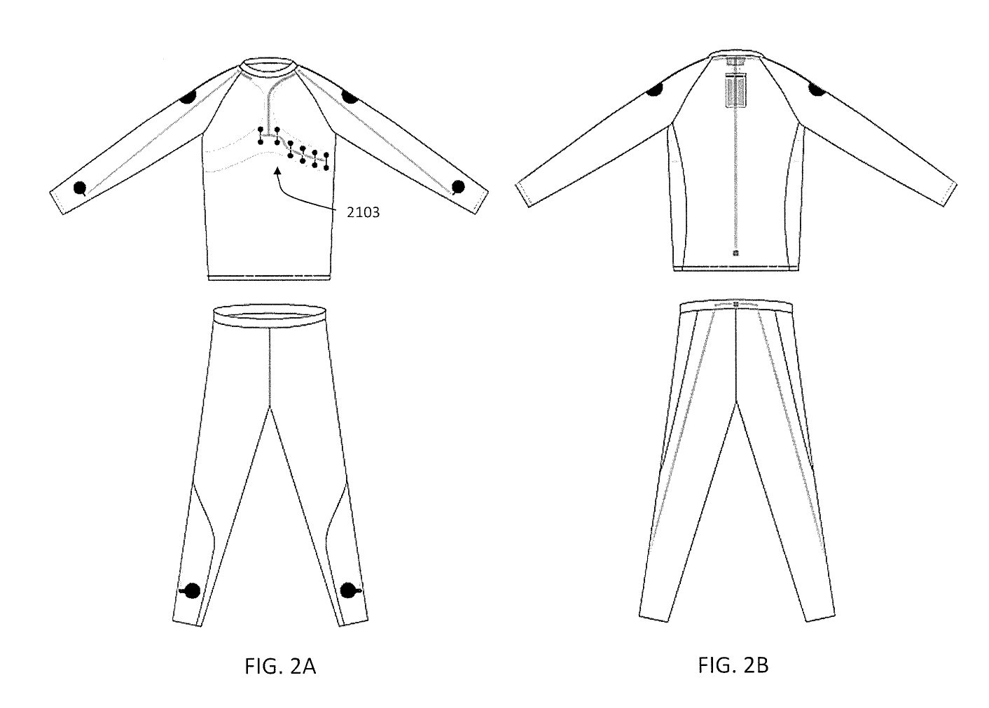

Also described herein are garments that may be used to effectively and continuously monitor electrocardiogram (ECG) signals. For example, a garment may be adapted to measure signals by including pairs of redundant traces between which the apparatus (e.g., garment, control/sensing module, etc.) may switch. In some variations the SMS and/or a sensor module may determine which set of electrodes between the redundant multiple electrodes to use in detecting a particular lead for an ECG. FIGS. 2A-2B, 3A-3B, and 4A-4B illustrate garments configured to measure ECGs. Each of these garments includes redundant leads (two or more) where each of the redundant leads can detect a signal from an electrode that may be used to determine an ECG signal for that lead.

The electrodes used to detect ECG signals may be formed of the stretchable conductive ink described herein. In some variations, the electrodes are printed, applied or formed on one side of the garment (e.g., the inner surface) and adapted to be in continuous contact with the subject's skin so as to measure ECG signals. Electrodes may be connected via conductive traces (formed by, for example, stretchable conductive ink and/or combinations of stretchable conductive ink and substrates such as Kapton with higher-conductance traces) to an SMS and/or sensor module. The SMS and/or sensor module may determine, e.g., based on the quality of the signal, which of the redundant traces to use/present for the ECG signal.

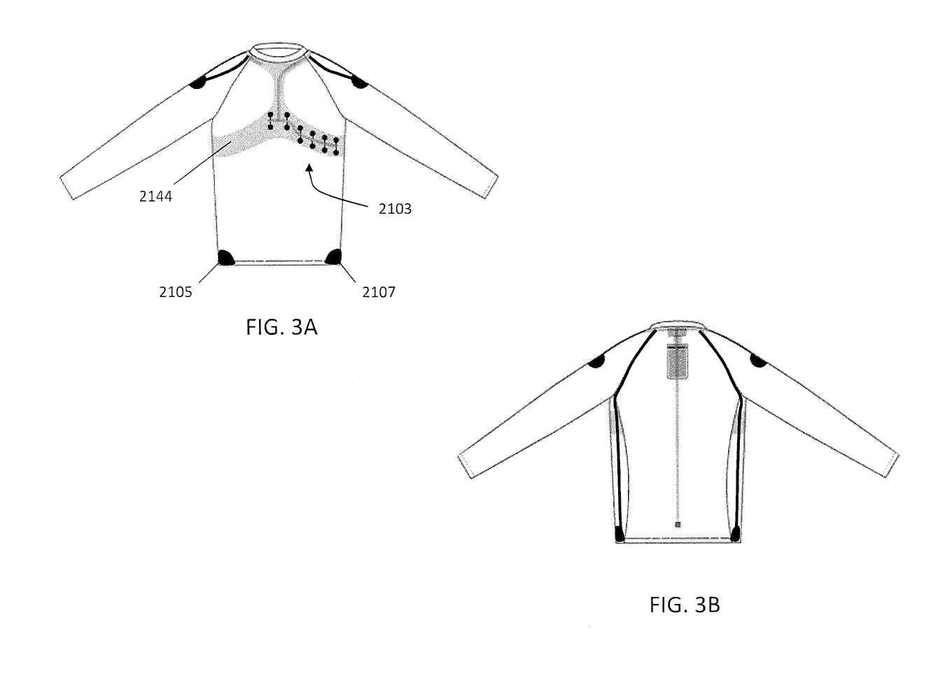

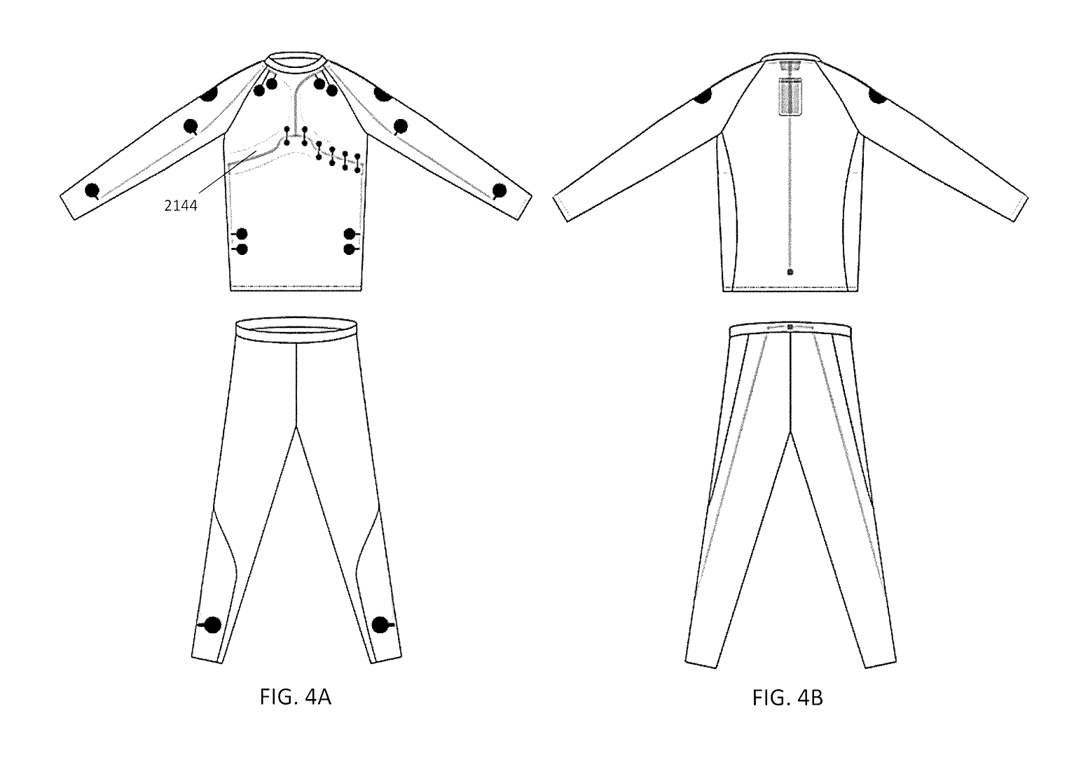

For example, in FIGS. 3A-4B, the electrodes 2103 are formed as a series of electrodes constituted by ink circles positioned in the standard points of the 12-lead EKG. On a garment (to be worn on the torso), the electrodes may be placed so that when the garment is worn the redundant (pairs) of chest electrodes are positioned corresponding to the V1-V6 positions:

TABLE 1
position of chest electrodes

| Electrode | Placement |
|---|---|
| V1 | 4th Intercostal space to the right of the sternum |
| V2 | 4th Intercostal space to the left of the sternum |
| V3 | Midway between V2 and V4 |
| V4 | 5th Intercostal space at the midclavicular line |
| V5 | Anterior axillary line at the same level as V4 |
| V6 | Midaxillary line at the same level as V4 and V5 |

Similarly leads may be placed at other locations on the shirt to measure the RL, RA, LL and LA leads (limb leads), corresponding to:

TABLE 2
Limb lead positions

| Electrode | Placement |
|---|---|
| RL | Anywhere above the ankle and below the torso |
| RA | Anywhere between the shoulder and the elbow |
| LL | Anywhere above the ankle and below the torso |
| LA | Anywhere between the shoulder and the elbow |

FIGS. 3A-3B show the limb leads for the legs positioned at the lower edge of the torso garment, which may be used even not wearing a separate pant. The limb leads in the garments shown in FIGS. 3A-3B and 4A-4B do not include redundant electrodes, however they may.

In any of the ECG-sensing garments, the electrodes may be held against the body for consistent/constant measurement (even during motion) by the structure of the garment, including by an additional harness region 2144 (e.g., yolk region), as shown by the shaded region in FIGS. 3A and 4A. This harness may be formed as a region supporting the ECG chest electrodes that is relatively more supportive (e.g., applying pressure/force) to hold the chest electrodes on/against the body, even during respiration and other body movements. For example, the harness region may be formed as an elastic corset (e.g., width: 2 cm on the sternum, 4 cm on the xiphoid line) running along the sternal line, then separating on the right and left sides of the xiphoid line, then on the back, then converging on the spinal cord and running up to the neck, then again separating into right and left sides around the neck, to finally converging on the sternal line. The material of the corset has to be extremely extensible.

The electrodes, and/or the region peripheral to the (e.g., chest) electrodes may include a silicone surface that helps hold the electrode(s) against the chest, and may also prevent the electrodes from slipping. For example, silicone may be located in an inner surface of the shirt, corresponding to the harness/corset position, along the horizontal line on both sides up to 5 cm beyond the midaxillary lines. This silicone may help ensure that the ink electrodes are fixed to the chosen position and do not move with patient's motion.

As mentioned, it is particularly helpful that the electrode include adjacent redundant electrodes. All of the electrodes (including the redundant electrodes) may be connected to the SMS and/or control module to detect ECG signals and the SMS and/or control module may decide which of the redundant signals to use (or in some variations to use the redundant signals to improve the overall signal quality, e.g., by selective filtering, averaging, or the like). In some variations the non-selected redundant signal may be ignored; in other variations the apparatus may be configured to store it for later analysis. Both pairs (or more than 2) of electrodes may have signals that may be stored, transmitted and/or processed; decisions about which of the redundant electrodes to use to generate an ECG may be made later.

### Sleep Monitoring Garment

Also described are garments configured to be worn to monitor a subject's sleep. Sleep monitoring may generally be used to measure sleep motion, respiration during sleep, body temperature (both core and regional), eye motion, and the like. Such indicators may be used to determine the sleep stage, sleep quality, sleep duration, etc. Any of the garments described herein may be adapted to determine sleep indicators and may therefore be worn while sleeping. Thus, these garments may be comfortable and adapted for use by a sleeping person.

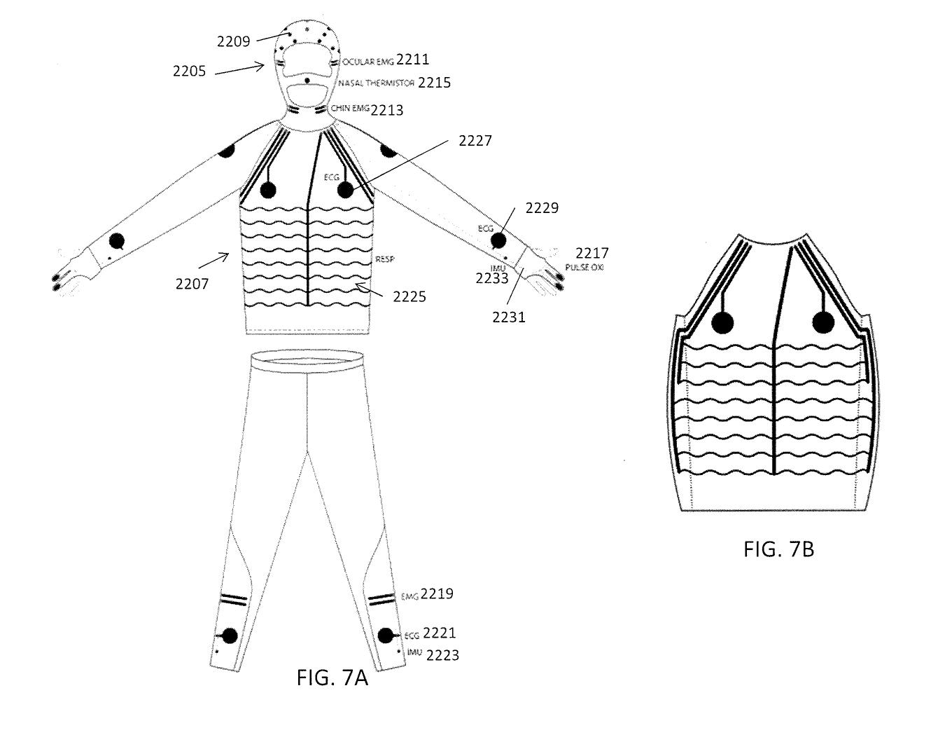

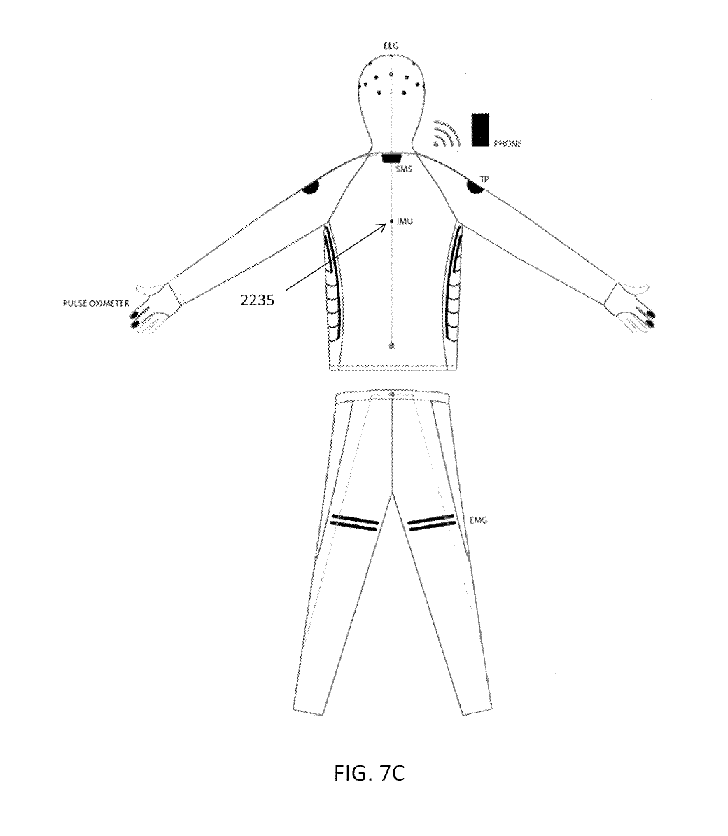

For example, in FIG. 7A, the front of the garment is shown, including a head cap/hood 2205 with sensors 2209 arranged to determine EEG (scalp electrodes on the inner surface of the hood), facial/ocular EMG (to detect eye movement), a nasal thermistor (detecting respiration) and chin EMG (detecting jaw motion, etc.). The hood may be integral with the shirt 2207 or it may be separately attached thereto. In any of the garments the various components (e.g., shirt, hood, gloves, pants, etc.) may be optional; individual garments or groups of garments may be used. The shirt may be similar or identical to the respiration and/or ECG sensing garments described above. In FIG. 7A and 7B, the torso region includes regional respiration sensors 2225 (stretchable conductive traces) for the anterior and lateral regions of the body, as well as EGC electrodes 2227 (though not all of the V1-V6 lead electrodes are included). The garment may also include pants including limb leads 2229 (for ECG detection) and/or EMG sensors 2219 to detect leg movement/twitch. Full or partial gloves 2231 may also be included and may measure blood oxygenation 2217 (e.g., pulse oxygenation) at the extremities (e.g., fingers).

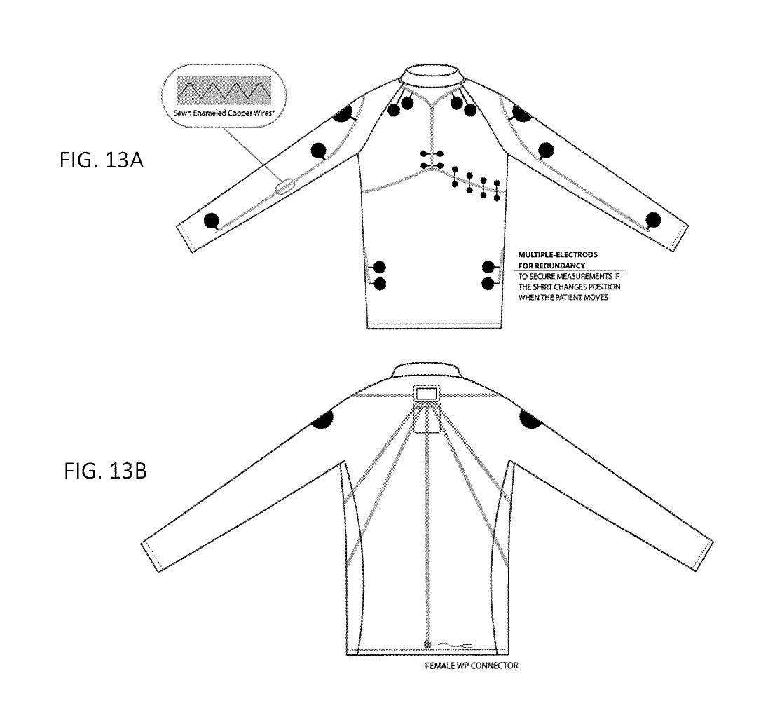

FIGS. 7A-7C illustrate one variation of a garment that may be formed as described herein and may include a plurality of sensors for determining sleep parameters. For example, in FIG. 7A, the front of the garment is shown, including a head cap/hood 2205 with sensors 2209 arranged to determine EEG (scalp electrodes on the inner surface of the hood), facial/ocular EMG (to detect eye movement), a nasal thermistor (detecting respiration) and chin EMG (detecting jaw motion, etc.). The hood may be integral with the shirt 2207 or it may be separately attached thereto. In any of the garments the various components (e.g., shirt, hood, gloves, pants, etc.) may be optional; individual garments or groups of garments may be used. The shirt may be similar or identical to the respiration and/or ECG sensing garments described above. In FIG. 7A and 7B, the torso region includes regional respiration sensors 2225 (stretchable conductive traces) for the anterior and lateral regions of the body, as well as EGC electrodes 2227 (though not all of the V1-V6 lead electrodes are included). The garment may also include pants including limb leads 2229 (for ECG detection) and/or EMG sensors 2219 to detect leg movement/twitch. Full or partial gloves 2231 may also be included and may measure blood oxygenation 2217 (e.g., pulse oxygenation) at the extremities (e.g., fingers). Additional embodiments of garments that can be used for sleep monitoring are illustrated in FIGS. 13A-21B.

The SMS and/or sensor module may be adapted to process and/or analyze the sensor inputs and to provide a report on the sleep status (or status over time) for the individual wearing the garment.

In general, these devices may be useful for a sleep lab or home sleep lab. They can record all of the signals usually included in polysomnographic analysis, including respiration, e.g., in a simplified way; only on the anterior and lateral part of the shirt; rib cage and abdominal part, 4 quadrants, may be needed to know when you have paradoxical motion. It is helpful that you have both upper and lower, but may also help to have right/left as well. The use of ECG in the upper part of the torso with a simplified (e.g., 2 electrodes and the wrists and legs) configuration is also helpful. The garment's sensors may again include redundancy as discussed above to have the best and most reliable ECG. In particular, heart rate is used, which may not require a full ECG. EMG recordings (electromyographic electrodes) may be formed of the stretchable conductive ink and may be located in different positions. For example, on the chin, the lower (muscle), which may be helpful for use in polysomnogrphic MG. In addition, ocular EMG may be helpful for detection of REM and other sleep stages. As mentioned a thermistor (temperature sensor at the level of the nose) may be used to detect airflow through the nose, similar to what is done with sleep lab. IMUs (inertial measurement units) may be used on the arms and legs to detect limb motion. Also, an IMU 2235 may be located on the back of the garment, which is useful for detecting the patient's position (rolling over, supine, prone, on side, etc.), and may detect restlessness.

### Expandable Supports

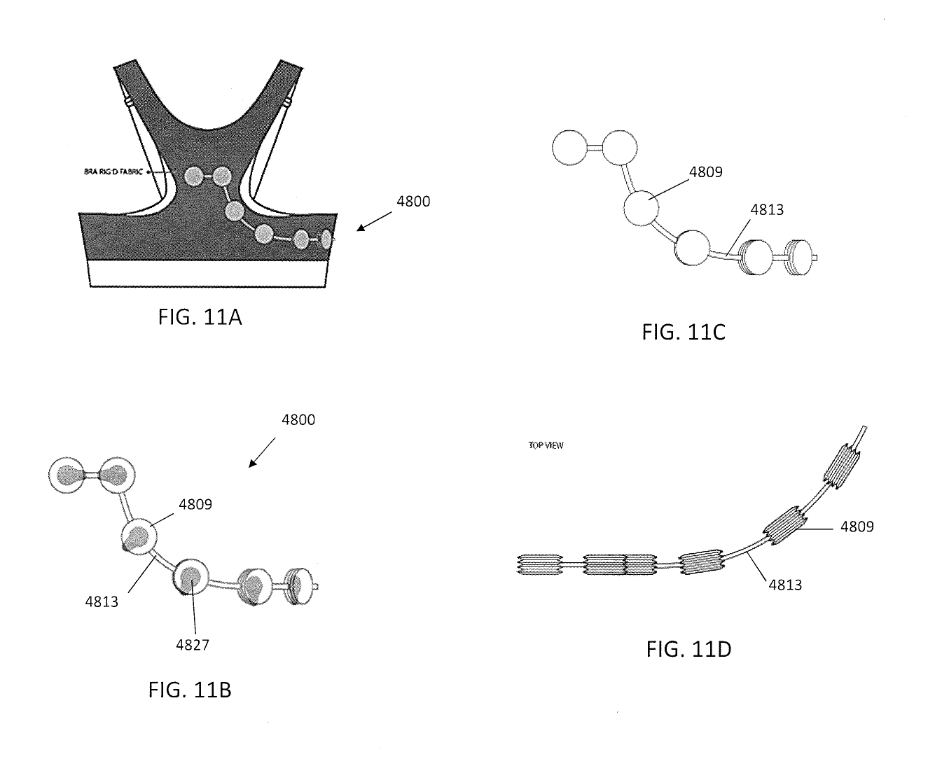

Any of the garments described herein may include additional support structures (e.g., expandable supports) to help secure the sensor(s) against the body. Such support structures may be expandable, and may improve the contact between the physiological monitoring garments disclosed herein and the skin of the wearer. For example, the chest anatomy can prevent a sensor on the physiological monitoring garment from making good electrical contact with the chest of the patient as described herein. A support garment, which may be generally referred to as an over-garment, such as a harness, can be worn over the physiological monitoring garment to provide pressure to improve electrical contact between the electrodes on the garment and the chest of the wearer. These harnesses (support garments) may be particularly useful with male wearers having large pectoral muscles and female wearers having large breasts. The support garment can include a strap or tie, and may be sized to hold (and apply force to keep) a portion of the physiological monitoring garment (e.g. sensors/electrodes) against the body of the user. Also described are structures that are integrated with these devices to apply force to keep the sensor(s) on the garment pressed firmly against the subject. Such integrated device may be referred to as integrated support structures. In some embodiments the support structure is a self-expanding structure. In particular, integrated support structures may be expandable, including inflatable, elements. Examples of support garments and portions of support garments are shown in FIGS. 11A, 14-18 and 48A-H.

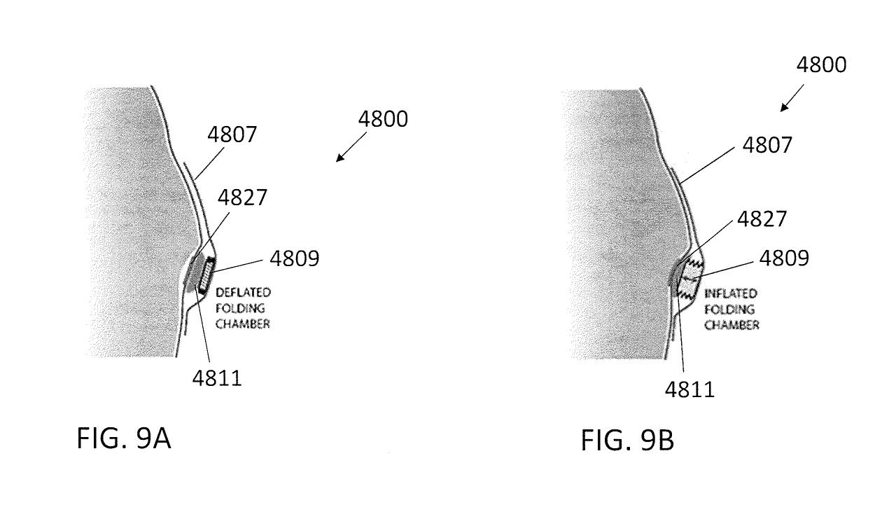

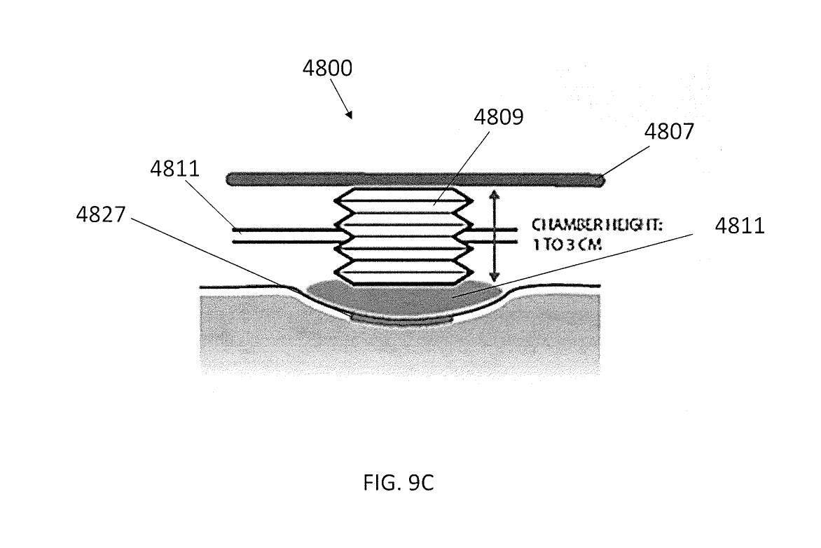

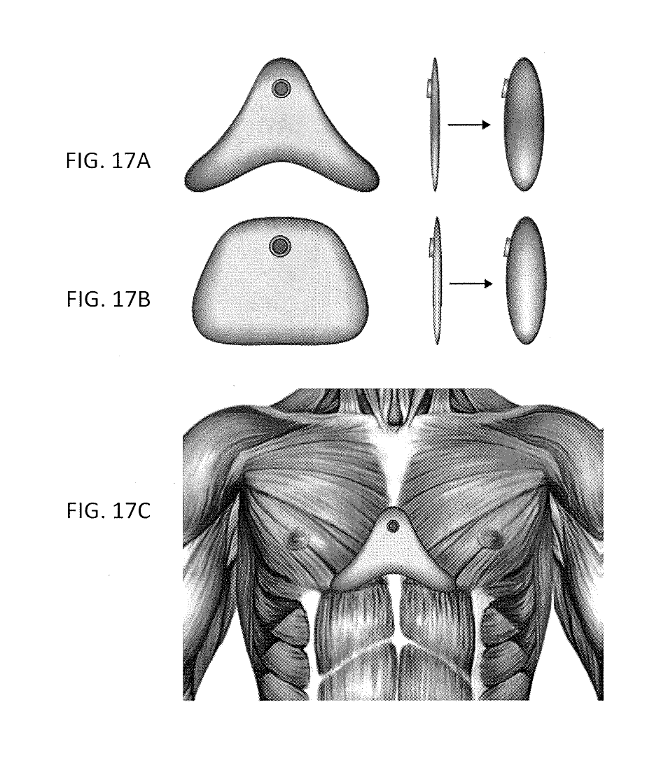

A support garment (e.g., harness) can be separate from the undergarments (e.g., compression garments) described herein, or they may be completely or partially integrated into the garment. A support garment and/or integrated support structure (expandable support structure) can be sized and shaped to fit the anatomy of the user. For example, the support garment and/or support structure can be designed to fit with the chest anatomy of the wearer. The support garment can be sized and shaped based on the gender of the wearer. For female wearer's a support harness can be designed to hold the support structure between the breasts of the patient. Examples of combinations of support harnesses and support structures that can be used for the support garment are illustrated in FIGS. 9A-12A, 14A-14D and 15A-15C. For male garment wearers a support strap can be used instead of a harness. Examples of male support straps and support structures are illustrated in FIGS. 16A-16C and 17A-17C.

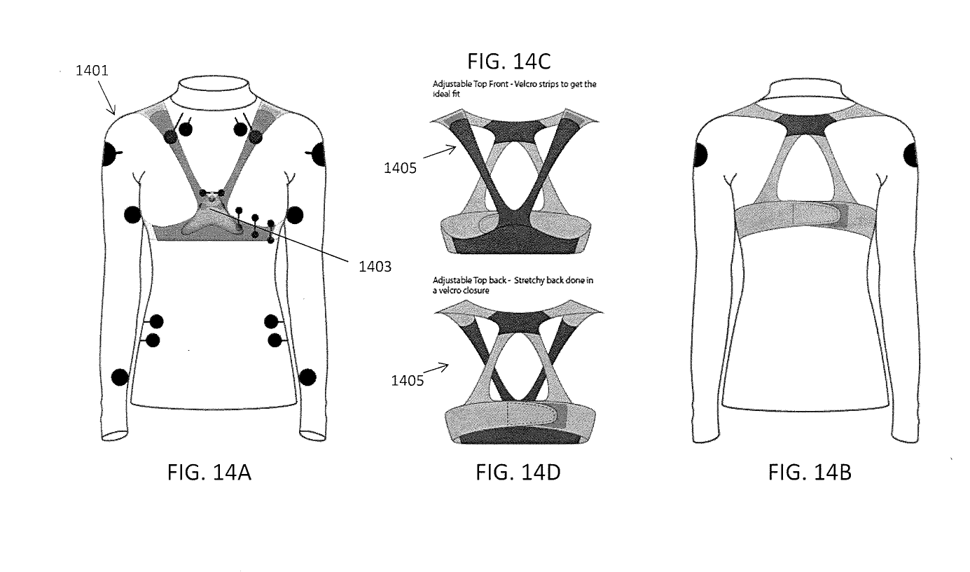

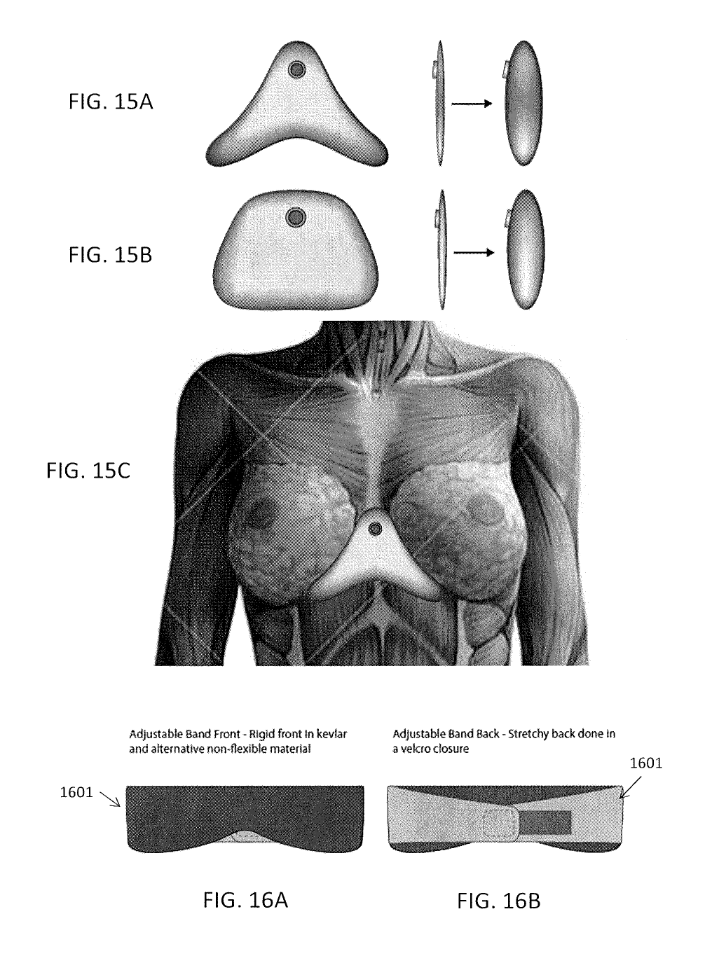

FIGS. 14A-14B illustrate a support garment 1405 from FIGS. 14C-14D worn over the physiological monitoring garments 1401 illustrated in FIGS. 13A-13B. An additional support structure (integrated into either the monitoring garment 1401 or the support harness 1405 in this example) may also be used. In this example, the support garment is shaped as a harness or sports bra-type configuration to hold the second support structure 1403 securely against the chest and sternum of the wearer. FIGS. 15A-15B illustrates expandable (e.g., inflatable, including self-inflating) support structures that can be used for female wearers. The support structures are shown in FIGS. 15A-15B in a front view and side views in non-inflated and inflated configurations. FIG. 15C illustrates the support structure of FIG. 15A engaged with a female chest. The illustrated support structures are inflatable and shaped to engage with a female chest to securely hold sensors on a smart garment against the chest of the wearer.

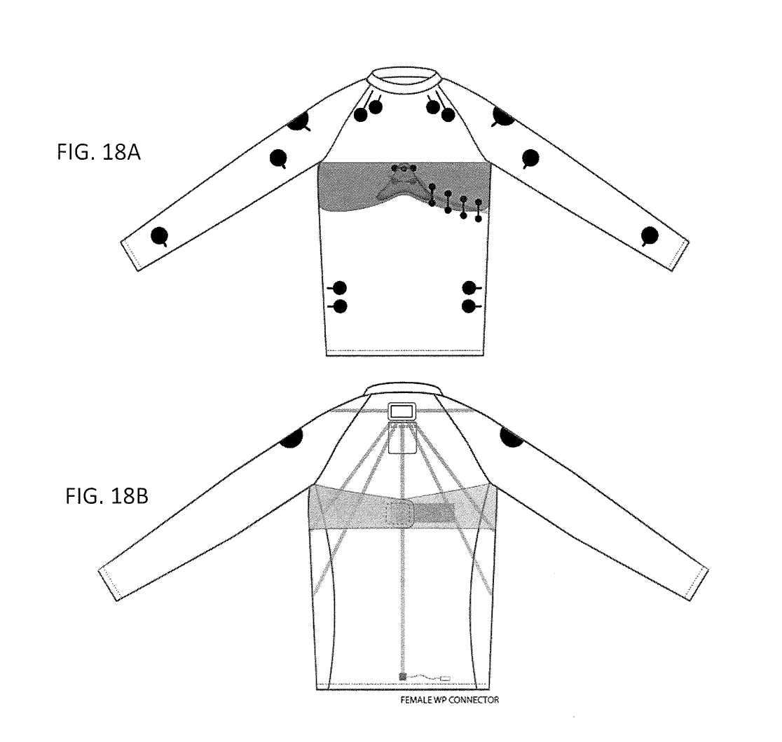

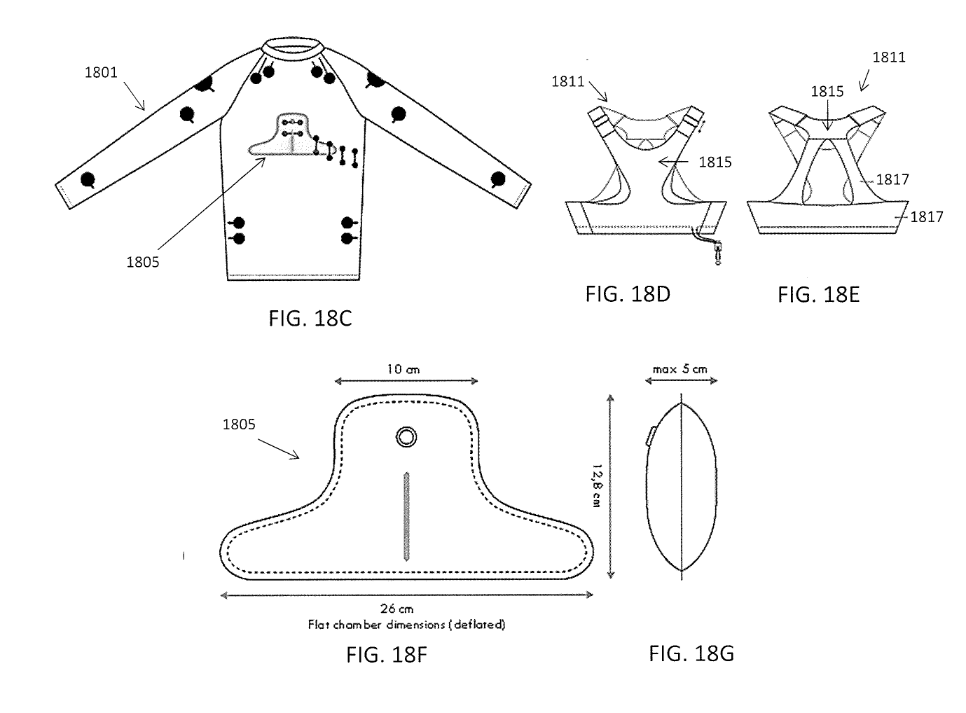

FIGS. 18A-18B illustrate the support garment from FIGS. 16A-16B worn over the physiological monitoring garments illustrated in FIGS. 13A-13B. FIG. 16A-16B illustrate the support strap 1601 having an optional rigid material in the front and an adjustable back material that can be made out of a stretchable material and can include Velcro. FIGS. 17A-17B illustrates support structures that can be used for female wearers. The expandable support structures are shown in FIGS. 17A-17B in a front view and side views in non-inflated and inflated configurations. FIG. 17C illustrates the support structure of FIG. 17A engaged with a male chest.

The support structures shown in FIGS. 17A-17B and 15A-15B provide inward force to compress the sensors on the physiological monitoring garment against the chest of the body to improve the electrical contact between sensors and the chest. In some embodiments the support structure can be expandable (including, but not limited to inflatable) to provide the desired structure to contact the monitoring garment. The support structure can be self-inflating in some embodiments. A self-inflating material can be used within the support structure such that the support structure automatically inflates when activated. In some cases the self-inflating material can be done via a foam material within the support structure, and/or via a chemical reaction. In some cases the chemical reaction can produce a gas or other material that can expand the support structure to conform to the chest anatomy of the patient. In some variations the support structure is a local pad or compressible material that can be held in place by the compression garment and/or the support harness.

In some embodiments the pressure applied by the support structure can be selected by the user.

In some embodiments the support garment and support structure can include sensors and a control system to provide the desired pressure level to the physiological monitoring garment.

The support garment, e.g. harness, strap, bra, etc., can include Velcro, adjustable straps, and other adjustable parameters so that the wearer can tighten the harness such that it provides the desired fit and support to improve the electrical contact of the chest sensors/electrodes.

In some embodiments the support garment and support structure can communicate electronically with the physiological monitoring garment and/or an external computing device.

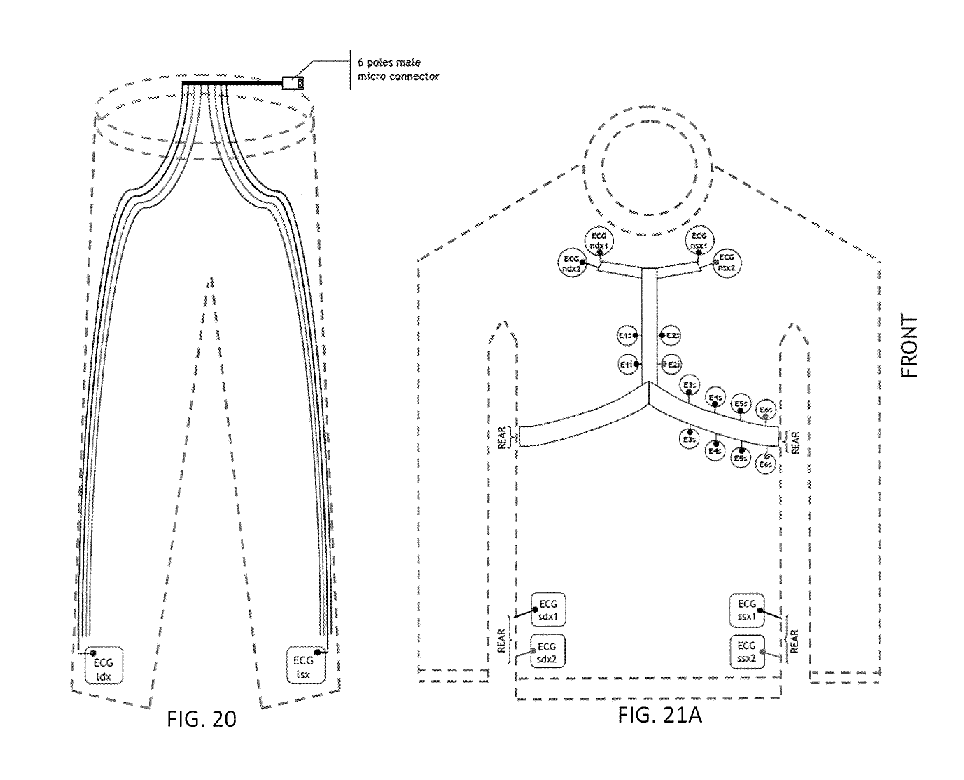

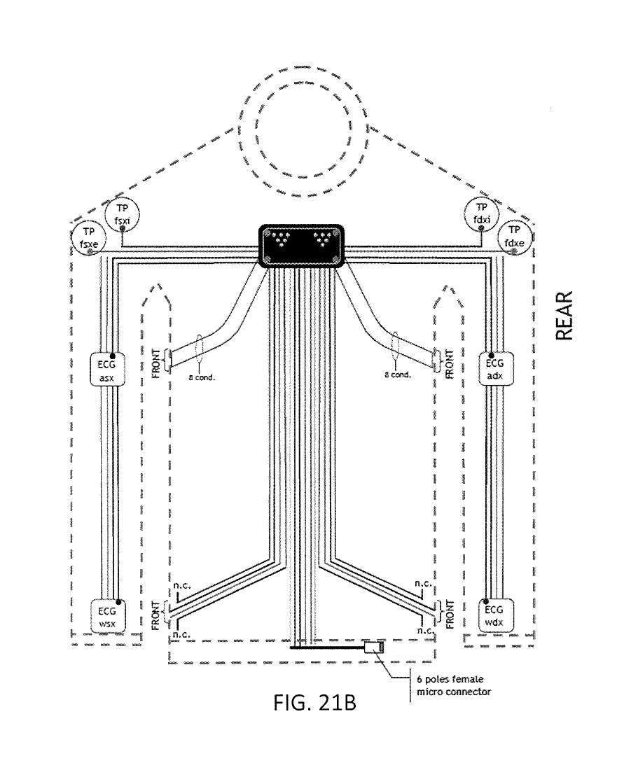

The support garment can be used with any of the physiological monitoring garments disclosed herein. In some embodiments the support garment is used with a compression shirt and pants having the wiring illustrated in FIGS. 20-21.

Returning now to FIGS. 18C-18E, a system including a sensing device configured as a compression garment 1801 as described above (e.g., for detecting ECG, as shown in FIGS. 2A-2B, 3A-3B and 4A-4B), may be worn directly against the patient's skin. A support structure (e.g., pad, expandable support member) 1805 may be used with the compression garment of the sensing device 1801. The support structure maybe located over the mid-pectoral region for applying pressure to hold the electrodes integrated onto the inner surface of the garment against the skin in the proper locations. The support structure may be expandable (e.g., inflatable) to allow comfortable and effective use with a variety of user body types. In some variations an additional support garment 1811 may be used to help secure the electrodes (and in some variations the support structure 1805) against the skin. The support garment 1811 in this example, shown in FIGS. 18D (front) and 18E (back), is a harness having a pair of straps that fit over the shoulders, and a central region that can push against the electrodes of the sensing device. The support garment may include relative rigid regions 1815 connected by relatively elastic regions 1817. FIGS. 18F (front view) and 18G (side view, inflated) show a larger view of the support structure 1805, with exemplary dimensions. The support structure may be attached to an outer or inner region of the sensing garment, so that it does not interfere with the measurements from the electrodes, but helps keep them pressed against the subject's chest. In some variations the support structure is integrated into the harness (support garment) such as the one shown in FIGS. 18D-18E.

FIGS. 9A-12B illustrate another example of a support garment in which a plurality of expandable supports are used to provide pressure against one or more sensors and thus holding the sensors against the user's skin to ensure that the electrodes make proper electrical contact with the wearer's skin. FIGS. 9A and 9B show one example of a single, discrete expandable support 4800. This example is an inflatable support, show in FIG. 9A in a deflated state and in 9B in an inflated state. Expandable/inflatable support structure 4800 in this example may be integrated into a lower (under) garment body to which the sensor(s) is/are attached, or it may be part of a separate support garment (overgarment) to be used with the undergarment having the sensors. The sensors are typically flexible, e.g. in FIG. 9A the sensor is a flexible electrode 4827 that is also in contact with a compression layer 4811. In any of the systems described herein, an intermediate (e.g., compression) layer may be included, e.g., between the expandable member and the sensor, which may increase comfort, e.g., by distributing the force applied by the expandable member more uniformly. The expandable member is positioned (“sandwiched”) between the electrode 4827 and a backstop formed at least in part of a second fabric that is a backing directing the expansion against the wearer. The backing in FIG. 9A is outer fabric structure 4807, and may include additional supporting members (wires, etc.). Inflatable member 4809 is located in between compression layer 4811 and backing 4807. Flexible electrode 4827 may be incorporated with/into the support structure 4800 portion of garment system, or the fabric body and sensor to which the fabric body is attached may be worn beneath the support structure 4800. There may be a series of flexible sensors (e.g., electrodes) such as individual flexible electrodes associated with particular regions of the wearer's body for monitoring physiological parameters. In the case where there is an array of electrodes, support structure 4800 may contain a corresponding array of expandable (e.g., inflatable) supports 4809. A single inflatable support 4809 may be associated with each electrode in the array series to ensure that adequate contact is achieved between the electrodes and the wearer's skin.

Compression layer 4811 may be flexible and compressible, such that when the inflatable member 4809 is in an inflated state, compression layer 4811 conforms to flexible electrode 4827 and firmly presses flexible electrode 4827 against the wearer's skin. As mentioned, variations having both a flexible electrode 4827 and a flexible compression layer 4811 may have improved contact between flexible electrodes 4827 and the wearer's skin when pressure is applied to the expand the expandable support. Compression layer 4811 can be any suitable material such as foam, rubber, fabric, gel, and so forth.

In FIGS. 9A-9C, the outer structure or backing 4807 may be a second fabric that is a more rigid material than the fabric (first fabric) to which the sensor is attached, such that when worn, it provides enough support and structure from which inflatable member 4809 can push away from and exert enough force against compression layer 4811, which then, in turn, may exert an appropriate amount of pressure against flexible electrode 4827 to make suitable contact between flexible electrode 4827 and the wearer's skin. Outer structure 4807 may be incorporated into a support garment, or it can be a strip of material that a wearer can attach to his or her body, both to be worn under clothing.

Inflatable expansion support 4809 may be inflated or deflated as shown in FIGS. 9A and 9B. When inflatable support 4809 is deflated (FIG. 9A), the flexible electrode is not help/pressed against the skin, as shown. When inflatable member 4809 is inflated, it expands in a direction that is perpendicular to the surface of the sensor (4827), and exerts force against compression layer 4811 which in turn exerts pressure against electrode 4827 to hold it secure against the wearer's skin. FIG. 9C shows inflatable member 4809 with outer material 4807 removed. In this embodiment, the expansion of inflatable member 4809 is one directional, namely, along an axis that simultaneously exerts a largely perpendicular force against the outer structure 4807 and compressible layer 4811. As FIG. 9C indicates, inflatable member 4809 can inflate up to three centimeters.

In other examples, it is also conceivable that the inflatable member can expand multi-directionally as well. Furthermore, while the inflatable members shown in figures are mainly cylindrical in shape, in other examples, the shape of the inflatable members may be any suitable three dimensional shape, either symmetrical or asymmetrical. In FIGS. 11C and 11D, a series of single expandable supports 4809 are arranged in a line; each expandable support may be associated with a corresponding flexible electrode, as illustrated in FIG. 11B. In other examples multiple inflatable members, in insolation from each other or fluidly connected, can be used to ensure that the flexible electrode makes good contact with the wearer's skin. For example, the inflatable supports used to support a single sensor may be smaller in size than those used where one inflatable member is associated with multiple electrodes.

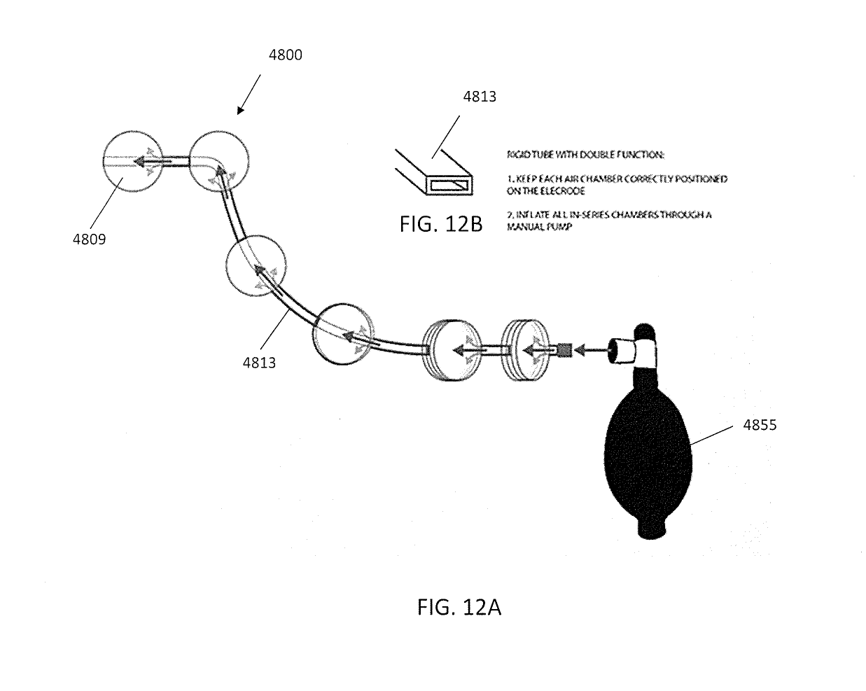

FIG. 12A shows expandable (e.g., inflatable) supports 4800 where inflatable supports 4809 are fluidly connection through a single inflation control line, connector 4813. An exemplary enlarged view of connector 4813 is shown in FIG. 12B. In this example, a pump 4855 may be included as part of the garment system (e.g., as part of the support or over garment or as part of an integrated garment) to inflate/deflate the inflatable supports when required. In some cases, a user can manually control the level of expansion of the inflatable members via an expansion control configured to control, e.g., the amount of inflation with the support structure, or in other cases, the means for controlling inflation of the inflatable members is contained within an overall controller. It is also possible to include sensors with the inflatable members such that the inflatable members automatically inflate and deflate based upon the amount of contact between the electrodes and the wearer's skin. It may be desirable to have sensor controlled inflatable members in a situation where the wearer is exercising and wishes to monitor a particular physiological parameter during exercise without having to stop and adjust the inflatable members to ensure contact between the electrodes and his skin.

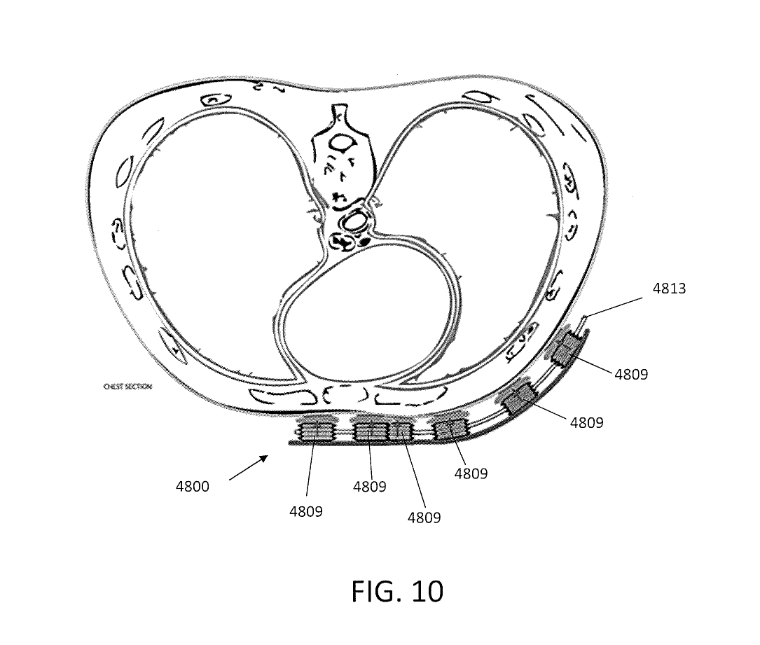

An expandable support can be incorporated into any of the previously described support garments described. FIG. 10 shows a series of inflatable electrodes incorporated into one type of support garment. The inflatable chambers can be in fluid connection such that other, such that all connected inflatable members are inflated and deflate in synchrony. Alternatively, in some examples, some but not all of the inflatable members are in fluid contact with each other. In other examples, multiple inflatable members can be isolated from other inflatable members for a particular electrode and individually inflated. In the embodiment shown in the figures, a single expandable support is associated with an electrode, but it is also possible that multiple expandable supports can be associated with one flexible electrode. Further, multiple expandable supports associated with different flexible electrodes along a support structure can also be fluidly connected throughout the sensor region and adjustable via one main control or the multiple expandable supports associated with different sensor regions can be isolated from one region to another and separately controlled.

### Garment Wiring Arrangements

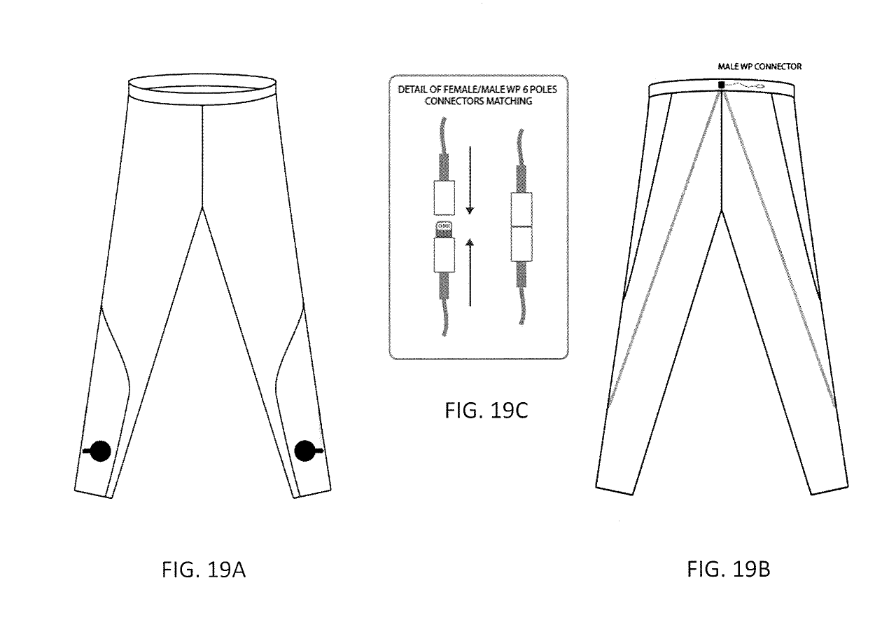

Various wiring arrangements can be used with the garments disclosed herein. Examples of wiring arrangements are illustrated in FIGS. 19-21.

FIGS. 19A and 19B illustrate front and back views of pants. FIG. 19C illustrates an exemplary connection between the garments disclosed herein, for example between a shirt and pants. For example, the pants and shirt can each include a connector with six or more poles. One connector can have a male configuration and the other connector can include a female connector. The male and female connectors are arranged to engage with each other. The illustrated pants in FIG. 19B include a male connector and the shirt illustrated in FIG. 13B includes a female connector. In some embodiments the male/female connectors can be reversed.

FIG. 20 illustrates a wiring diagram for pants in accordance with some embodiments. The pants include a sensor for measuring the heartbeat reading of the wearer on the left leg and a sensor for measuring the heartbeat reading of the wearer on the right leg. The illustrated pants include wiring from the sensors along the pants legs to the male connector.

FIG. 21A illustrates a wiring diagram for the front of a garment in accordance with some embodiments. FIG. 21B illustrates a wiring diagram for the back of a garment in accordance with some embodiments. The illustrated ECG wsx is a sensor for heartbeat reading, wrist, left side. The illustrated ECG wrx is a sensor for heartbeat reading, wrist, right side. The illustrated ECG asx is a sensor for heartbeat reading, arm, left side. The illustrated ECG asx is a sensor for heartbeat reading, arm, right side. The illustrated TP fsxi is a touch point, front position, left side, internal. The illustrated TP fsxe is a touch point, front position, left side, external. The illustrated TP fdxi is a touch point, front position, right side, internal. The illustrated TP fdxe is a touch point, front position, right side, external. The illustrated Microconn6p has 6 poles WP female connector for Compression Pants CP connection. The illustrated ECG ndx1 is a sensor for heartbeat reading, neck, right side, n.1. The illustrated ECG ndx2 is a sensor for heartbeat reading, neck, right side, n.2. The illustrated ECG nsx1 is a sensor for heartbeat reading, neck, left side, n.1. The illustrated ECG nsx2 is a sensor for heartbeat reading, neck, left side, n.2. The illustrated E1s is a sensor for heartbeat reading, chest, upper, n.1. The illustrated E1i is a sensor for heartbeat reading, chest, lower, n.1.The illustrated E2s is a sensor for heartbeat reading, chest, upper, n.2. The illustrated E2i is a sensor for heartbeat reading, chest, lower, n.2. The illustrated E3s is a sensor for heartbeat reading, chest, upper, n.3. The illustrated E3i is a sensor for heartbeat reading, chest, lower, n.3. The illustrated E4s is a sensor for heartbeat reading, chest, upper, n.4. The illustrated E4i is a sensor for heartbeat reading, chest, lower, n.4. The illustrated E5s is a sensor for heartbeat reading, chest, upper, n.5. The illustrated E5i is a sensor for heartbeat reading, chest, lower, n.5. The illustrated E6s is a sensor for heartbeat reading, chest, upper, n.6. The illustrated E6i is a sensor for heartbeat reading, chest, lower, n.6.

### Wearable System For Detection of Emotion

Also described are garments configured to determine a wearer's emotional state. Self-reported emotional state tends to be inaccurate, subjective, and therefore limited in use. Garments that may include sensors detecting various parameters (both voluntary and involuntary parameters) may be used to determine a subject's objective emotional state.

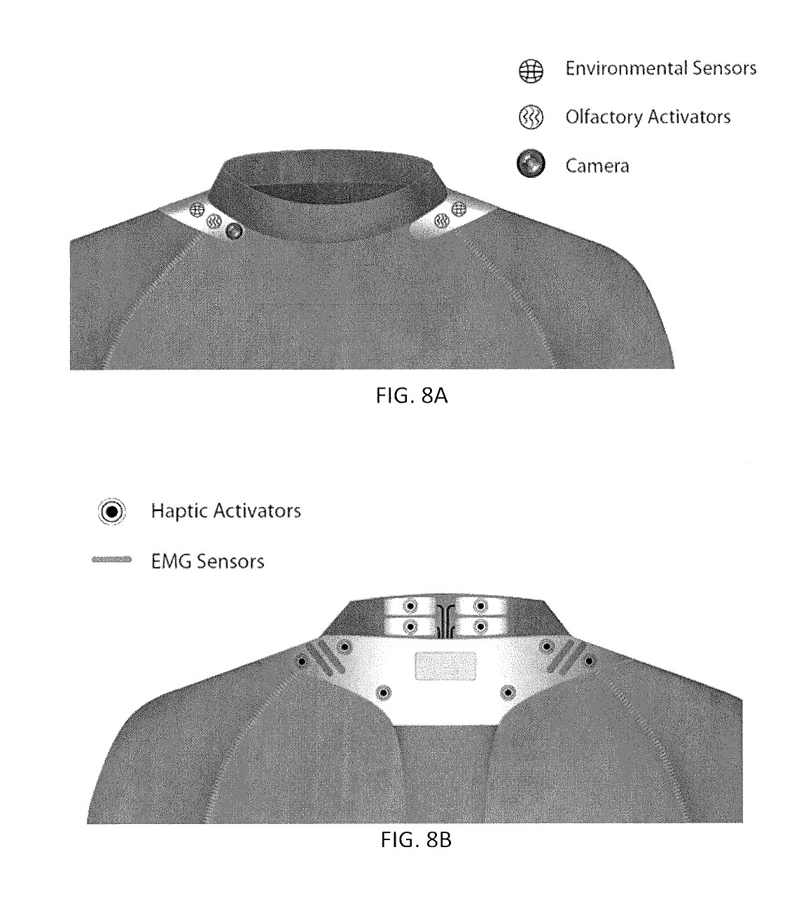

A garment may include a plurality of sensors (as described below and illustrated in FIGS. 8A-8B illustrate a collar that may be included as part of the garment and includes a plurality of sensors (any of which may be included or omitted) to detect parameters indicative of a wearer's emotional state. Sensors may include, for example: environmental sensors (detecting environmental temperature, humidity, etc.), camera(s) for visual detection, including light levels/intensity, audio detectors (e.g., detecting user voice volume, tenor, etc.). The collar may also include any of the other sensors mentioned herein and incorporated by reference (motion sensors, position sensors, acceleration sensors, etc.). In addition, the collar may include one or more outputs (haptic outputs) to provide output, including feedback, to the wearer. Haptic outputs may include olfactory (scent emitting) outputs, tactile output (vibration, pinch, etc.), and the like. The collars described and shown in FIGS. 8A-8B may be configured as an emotion communication receiver (ECR).

Any of the garments for detecting/monitoring emotion may include an ECR. An ECR may sits around the neck. In FIGS. 8A-8B the ECR is a collar that extends from the back, spreading above left and right trapeziuses, extending to the front lateral left and right sides of the neck without reconnecting on the front to facilitate the ‘sliding’ of the head through the collar of the ‘device’. The receptor in the ECR (collar) may house a communications/analysis module (sensor module) and may include connectors (e.g., female and male connectors) as well as sensors, haptic activators and mechanisms generating pressure, vibration, temperature-changes, tensing & relaxing inputs, olfactory-inputs, etc. The front side of the activator also houses smell and taste inducing activators as well as environmental sensors to determine the quality of the environment.

The ECR may transduce received communication of physiological measurements into physically embodied messages. As an example, a friend may send to the user (wearer) of the device her emotional state as measure by her device: the user's ECR may transduce the communication into a sensorial message such as a salute by applying pressure to his shoulders. Users may exchange sensorial messages such as salute touching the shoulder, hug, push, caress, cheer up, relax, etc. and have the option to respond, including: i) Ignore; ii) accept and salute back (with their own message); iii) reject (electrical discharge). Users can choose how to receive the messages between a) pressure (wide), b) pressure (narrow-puncture), c) pressure-message (Morse-like), d) vibration, e) temperature change, or the like (including combinations). Users may also choose not accept the “emotional” valence messages to preserve her/his privacy and/or may provide a feedback to improve the accuracy of the emotions-interpretation language.

### Stretchable Conductive Ink Patterns

The conductive ink described herein may be used to form flexible conductive traces (including electrodes). In some variations, these flexible conductive ink traces may be stretchable conductive ink traces. Any of the apparatuses described herein may include a stretchable conductive ink pattern. In general, the stretchable conducive ink may have a stretchability ranging from 5% to 200%, e.g., it may be stretched more than 2 times (200%) of its at-rest length without breaking. In some examples the stretchable conductive in can be stretched to more than 3 time (300%), more than 4 time (400%), or more than 5 time (500%) of its neutral, at rest length. The stretchable conductive ink patterns are conductive, having a low resistivity. For example, the bulk resistivity may be between 0.2 and 20 ohms*cm (and the sheet resistivity between about 100 to 10,000 ohms per square). The conductivity may be dependent upon the stretch, although it may stay within the ranges described above (e.g., between 0.2 and 20 ohms*cm).

Structurally, any of the stretchable conductive ink patterns described herein are typically made from a specified combination of an insulative adhesive and a conductive ink. In general, a stretchable conductive ink pattern includes a first (or base) layer of insulative and elastic adhesive and a layer of conductive ink, where the conductive ink includes between about 40% and about 60% of conductive particles (e.g., carbon black, graphene, graphite, silver metal powder, copper metal powder, or iron metal powder, etc.), and a gradient region or zone between the insulative, elastic adhesive and the layer of conductive ink. The gradient region is a combination of the conductive ink (e.g., conductive particles of the conductive ink) and the adhesive, in which the concentration of the ink (e.g., conductive particles) may vary with depth. In general, the gradient region may be a mixture of the conductive ink (e.g., conductive particles) and the adhesive wherein the concentration of conductive ink in the gradient region may be less than the concentration of the conductive ink in the conductive ink layer. The gradient region may be a continuous gradient of conductive ink (particles), e.g., it may be nonhomogeneous, or it may be a step gradient.

Typical conductive inks, such as those used for printed circuits and even flexible circuits, are not sufficiently stretchable to be used for garments, including in particular not for compression garments and may break or form discontinuities when used. Surprisingly, the combination of conductive ink, gradient region and insulative adhesive provides a conductive ink composite that is both conductive and highly stretchable/extensible. The composition of the conductive ink that may be used in as described herein generally includes: between about 40-60% conductive particles, between about 30-50% binder; between about 3-7% solvent; and between about 3-7% thickener. Further, the use of an intermediate, “gradient” region between the insulating adhesive and the conductive ink layer(s) has also been found to be important.

The conductive ink used and combined with the adhesive to form the conductive ink pattern typically has a low toxicity and hypo-allergenicity (e.g., a formaldehyde concentration lower than 100 ppm), and a resistance to damage from washing, including preservation of electrical and elastic properties following repeated washing cycles.

The gradient region may be functioning both to enhance the stretchability of the conductive ink, as well as enhancing the stability of the conductivity. Electrical conductivity is allowed by the upper region, while the high degree of mechanical stretching allowed (due to the adhesive) is enhanced by the lower layers. The incomplete mixing of the conducive ink and the adhesive found in the gradient region appears to result in a structure and composition that can be repeatedly stretched and released, while retaining the conductivity. Note that the resistivity of the composite may change with stretch (generally increasing resistivity with stretch), and this property may be used to detect stretch.

In general, the gradient region may be formed by combining the conductive ink and the adhesive before either one is completely dried, allowing them to combine to form the transition zone having the appropriate thickness. The composition of the ink (e.g., between about 40-60% conductive particles, between about 30-50% binder; between about 3-7% solvent; and between about 3-7% thickener) may determine the formation parameters of this overlapping (gradient) region.

An outer protective layer that insulates the conductive ink may be included when desired, e.g., when forming conductive traces, or patterning a sensor or electrode, though it may be left off contract regions of an electrode, for example. The resin (“primer”) may be one or more layers of insulating material that does not link with or mix with the conductive ink. For example, the resin material may be insulating and may also help protect from detergents and fluids (water) used for washing, as well as protecting from scratching, etc. In some variations the resin is an acrylate (e.g., acrylic resin). Aldehyde or acrylic (synthetic resins) may also be used. Any of the components (e.g., conductive ink, adhesive, and resin) may be applied by printing.

In some variations of the conductive ink structures described herein (e.g., traces, sensors, etc. formed of conductive ink, e.g., by printing directly and/or transferring to a fabric), the conductive ink comprises conductive particles, such as carbon black, coated mica (e.g., mica coated with antimony-doped tin dioxide), graphene, graphite, etc. The material may also include a base/binding material that functions to permanently bind to the fabric all the solid components contained in the ink. This binding material (binder) may be an acrylic water base, e.g., water-based polyurethane. The conductive ink material may also include a primer, that increases adhesion and compatibility between the various products applied and increase the resistance to washing process. The conductive ink may include an adhesive (e.g., glue, such as an acrylic, polyamide, etc.), that ensures the transfer of the conductive product to the fabric. Any of these conductive inks may also include a de-foamer to eliminate air and foam contained in the product, and a catalyst to allow the complete crosslinking of the binder. Additional additives may be included to increase the printability and the stability of the product. A thickener that thickens the liquid components contained in the product may also be included. Transfer of the resulting ink material may be obtained by a silkscreen print process as illustrated above. For example, a silk screening process may include a serigraphy frame type (from 24 wires up to 120 wires), and transfer supports films such a paper, cardboard, polyester, acetate, reflector, etc. The number of layers screened/applied may be from 1 up to 50 or more. The order of the layers applied may be sequential (and inverted when the material is to be transferred). For example, the primer may be applied as the next to last layer, with the adhesive being the last layer formed. The conductive ink may be dried, e.g., by IR oven, hot air blower or cold air blower. As mentioned above, this ink material (including the adhesive base) may be applied to any appropriate material, including, e.g., cotton, woolen, nylon, polyester, polyamide, Lycra, leather (natural or synthetic), plastic films, ESD fabric, etc.

The ink may be transferred to apply to a garment using a thermo press machine, e.g., by applying an application pressure from 2 bar up to 90 bar at an application temperature from 100° C. up to 250° C. for an application time from 5sec up to 50sec. The final polymerization may be performed by IR oven at a temperature from 50° C. up to 180° C. (e.g., using a conveyor belt speed from 0.1m/sec up to 5m/sec).

As mentioned above, the conductive ink patterns described herein may be any appropriate pattern, including traces (e.g., connecting various elements on the garment), sensors (e.g., touch point sensors, stretch/respiration sensors) or electrodes (EEG sensors, ECG sensors, EMG sensors, etc.). When used as a connector it may be combined with additional conductive connector elements, including, but not limited to conductive threads, stitched zig-zag connectors, conductive traces formed on a substrate such as Kapton, etc. Such combinations of conductive ink patterns and additional highly conductive materials may be particularly useful over longer lengths. In some variations the stretchable conductive ink material may be used as a trace or connector in regions where the garment will be stretched a lot.

In FIGS. 1A-AF, 2A-2B, 3A-3B, etc., the touchpoints and the traces connecting them to a sensor module (sensor manager) may be formed of a stretchable conducive ink composite including a layer of adhesive, an intermediate gradient region and a layer of conductive ink; the trace portion may be insulated, e.g., using a protective resin. The electrode forming the touchpoint portion may be relatively large with the connecting trace being smaller The trace only needs to extend a short distance. Touchpoint sensors are also somewhat insensitive to stretch of the garment/trace that might change the resistivity of the trace, because the signal from the sensor is a binary signal—e.g., touch or no touch. Similarly, a stretchable conductive ink trace (composite formed into a trace) may be used to connect to EKG electrodes. Typically a conductive ink pattern used as a trace may extend up to 30 cm or less (e.g., 25 cm or less, etc.), although longer traces may be used. Thus, for example, a conductive trace formed of a stretchable conductive ink pattern may be as long as or longer than 25 cm, with a width between 2 mm and up to 10 mm (an average of between about 0.6 to 0.5 mm). The length could be extended while remaining within a target conductivity/resistivity by increasing the thickness of the conductive ink pattern. In some variations it may be desirable to keep the length short. Respiratory sensors may be substantially longer, however, and may up to 22 mm wide, for example.

In some variations it may be useful to use conductive threads or other high-conductivity connectors. As described above, this may be used to form a stitched zig-zag connector (also referred to herein as a wire ribbon material). In this example, the conductive thread is stitched onto the garment in a wavy (e.g., zig-zag, sigmoidal, etc.) pattern that allows some stretching in the net direction of the stitching. As described above, respiration (sensors) traces may be formed of stretchable conductive ink patterns to take advantage of the change in conductivity with the change in resistivity with stretching of the conductive ink pattern. In this example, the sewn pattern of threads includes an approximately 35-40 degree zig-zag pattern allowed the stitch to elongate slightly with the fabric. In some example, the conductive thread is a metallic conductive thread. The angle formed at each turning point (in the wavy pattern) and the width of the pattern may depend upon the textile used. In general, the higher the stretchability of the textile, the smaller the angle. The number of threads may vary; in general, any number of threads may be used depending, for example, on the number of sensors and their pins that need to be connected. The threads are typically sewn directly on the garment. The electrical insulation of the thread may be obtained by an external coating on the thread (e.g. silicone, polyester, cotton, etc.) and/or by a layer of insulating adhesive, as described above. The thread connectors may also be used as part of a transfer as described above. For example, a conductive thread may be sewn on a band made on the same fabric of the garment and then transferred by a thermal process to the garment, e.g., using a layer of adhesive.

One or more conductive threads may be applied directly to a fabric (such as a compression garment) or to a transfer (e.g., patch of fabric or other material that is then attached to the garment). Conductive threads may be insulated (e.g., enameled) before being sewn. In some variations the conductive thread may be grouped prior to sewing onto a fabric or other substrate. For example, a plurality (e.g., 2, 3, 4, 5, etc.) of threads may be insulated and wound together, then stitched into a substrate, such as the compression fabric. For example, in one variation, an apparatus includes a garment having an IMU and two EMGs with inputs fed into circuitry (e.g., microchip) on the apparatus, including on a sensor module/manager. The components may be operated on the same electronic ‘line’, where the line is a plurality of electrically conductive threads that are combined together for stitching through the substrate. In one example, two microchips can be operated by the same ‘line’ made of 4 wires, where each wire is electrically isolated from each other. In stitching a material, the stitch may be formed of two sets of wires; one on top of the substrate and one beneath the substrate, as is understood from mechanical sewing devices; in some variations a stitch formed of conductive thread may include an upper conductive thread (or group of conductive threads) and a lower conductive thread (or group of conductive threads), where the upper conductive thread(s) is primarily on the upper surface and the lower conductive thread(s) are primarily on the lower surface (but one or either may pass through the substrate to engage with the other).

For example, a conductive thread may include a very fine (e.g., 0.7 millimeters gauge/thickness) ‘wire’ made of 4 twisted and enameled (thus electrically isolated from each other) wires covered with a binding solution (that is silicon or water based) or protected by a jacket, having a total diameter of about 0.9 millimeters. A conductive wire may be sewn in a wavy (e.g., zig-zag) pattern, such as a pattern having 45 to 90 degrees angles between the legs of the zig-zag, directly on a fabric or substrate. In some example, the pattern is formed on a substrate of material (e.g., fabric) and attached to the garment. For example, the substrate may be a 1 cm to 3 cm self-adhesive strip of fabric.

### ECG Monitoring Garment

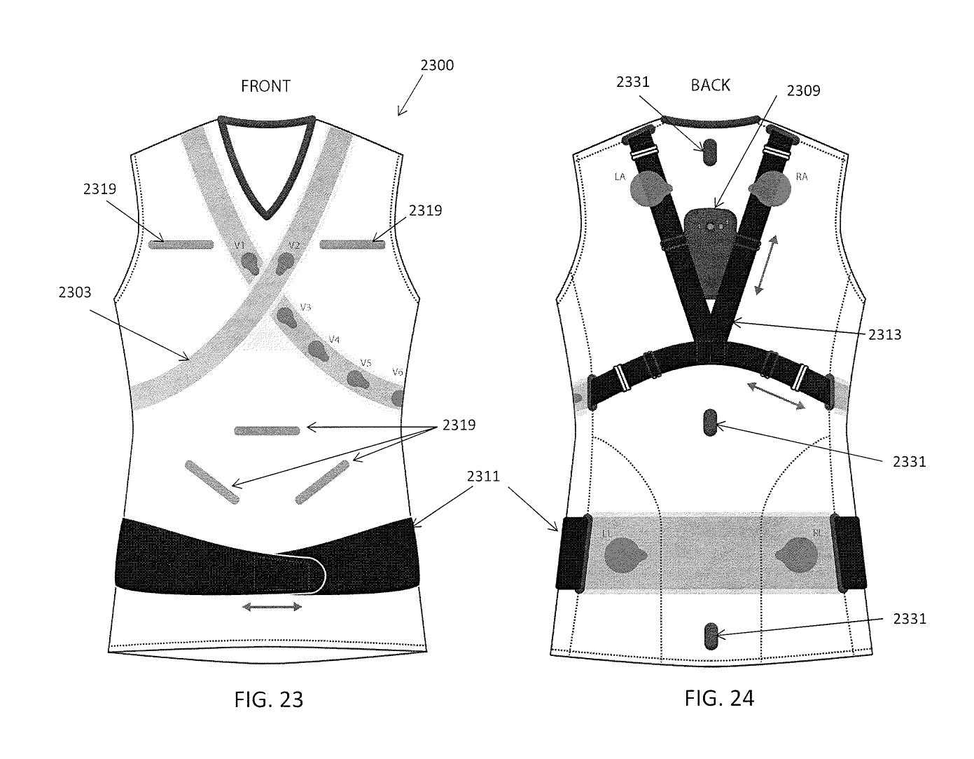

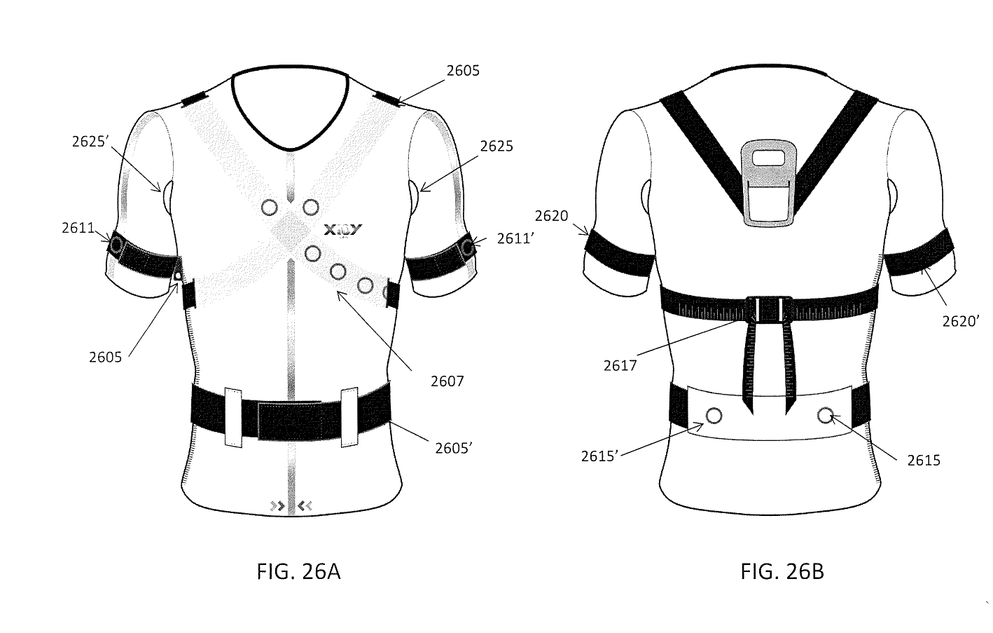

FIGS. 23 and 24 show front and back views, respectively, of an ECG monitoring garment that may include any of the features described individually above, including the expandable support(s). In FIG. 23, the apparatus (garment 2300) is configured as a sleeveless shirt to be worn on a male or female torso, against the subject's skin so that the electrode contacts (which are exposed on the inside of the garment, not shown) may contact the subject's skin. The electrode contacts may be the portion of an electrode that contacts the user's skin or it may be a conductive material in electrical contact with the electrode. A single integrated garment may be used, as shown in FIGS. 23 and 24 (and also in the example of FIGS. 26A-26B described below), showing a single shirt without sleeves. The garment may include sleeves, and/or may be configured as a unitard, body suit, etc. (e.g., may include a pants or legging portion). A sleeveless garment may be easier to wear, particularly for older subjects, and may provide less interference with movement (e.g., arm movement).

In general, the apparatus may incorporate an adjustable harness into the garment (e.g., shirt, unitard, etc.). Thus, three or more rough garment sizes may be provided (e.g., small, medium, large), while allowing more fine sizing to fit and/or to keep the electrodes in good and consistent contact with the skin. The harness portion may comprise a ribbon or strap (which may be an elastic strap/ribbon) that may be slideable coupled into channels within the shirt, as illustrated. For example, in FIG. 23 the X-shaped harness crossing over the front of the wearer's chest 2303 is held within a channel (e.g., pocket, cuff, etc.) within the garment. In some variations the elastic is slideable within the channel. In some variations the elastic is not slideable at least over part of the garment (e.g., the front). The garment may be specific for men or for women. For example, the women's garment may be adapted to provide support for the woman's cleavage, or for being worn with such support (e.g. a bra). Thus, the garment may include additional support structures, including an underwire, webbing, straps, etc.

The straps 2303, 2313 may be behind or between the electrodes, which include an exposed surface on the inside of the garment. The straps may aid the electrodes and/or sensors adhering to the skin.

In some variations the channels within the garment for the straps (shown in the front view of FIG. 23) are formed by sewing, gluing, welding, or otherwise attaching a fabric strip to the overall body of the garment, to form the channel. For example the channel may be formed by welding the material (either the strap directly, or a material forming a channel) to the inner or outer surface of the shirt front, as shown in FIG. 23. Alternatively or additionally, the garment may include two layers (an outer layer and an inner layer), with the strap traveling between the two; the two layers may be partially fused or connected, defining channels or passages between the two layers for the strap(s). The straps may be exposed over at least a portion of the garment, as shown in the back view of FIG. 24, and the straps may be adjusted (e.g., tightened and loosened), which may aid in assuring that the electrodes adhere to the wearer's skin without discomfort.

In FIG. 24, the garment includes straps that may be adjusted from the back of the garment. These straps may be fastened by Velcro or by buckles, snaps, etc. The back of the garment also includes an attachment region 2309 near the center between the wearer's shoulder blades for attaching a controller (e.g. phone) (not shown). A phone may be mounted so that it c can interface with the other electrical components on the garment. The phone may be mounted in a manner that allows the user to capture images of what is behind the user. In this example, the apparatus also includes a waist strap 2311 closer to the waist region of the wearer wearing the garment. This waist strap may help secure additional electrodes (e.g. the left leg, LL, and right leg, RL, electrodes). The leg electrodes may be integrated into the back or sides of the garment (shown near the kidney region of the wearer in FIG. 24). Similarly the arm electrodes (left arm, LA, and right arm, RA) may be integrated in to the upper shoulder region or back of the garment, so that the electrodes contact the upper back region of the wearer when worn as described. As will be described in FIGS. 26A-26B, the arm electrodes may be positioned on each of the user's arms (e.g., near the biceps) and may be secured in place with another elastic and/or adjustable strap. In FIGS. 23 and 24, the upper torso straps 2313 in the back may hold or secure these arm (LA, RA) electrodes at the shoulder against the wearer. Along the front of the garment, the six precordial leads (V1-V6) may be positioned so that the strap integrated into the garment is behind them (e.g., on strap supports V1, V3-V6 and the other, crossing, strap supports V2; similarly, one strap may support V1 and V2 while the other supports V3-V6; one strap may support V2-V3 while the other supports V1 and V4-V6).

Any of the straps, including the waist strap 2311, may be belting (e.g., may be adjustably fastenable), and may be an elastic or flexible material, e.g., that is stretchable from 0.1 to 5× its normal length with the application of a small amount of force. In some variations, the straps can be open and adjusted, e.g., by securing Velcro or other securements (snaps, clips, rings, etc.). For example, in FIG. 23 the belt strap (waist strap 2313) travels over the top of the garment over all or a portion of the front, but is integrated into a channel in the back, and is adjustably securable by a Velcro material.

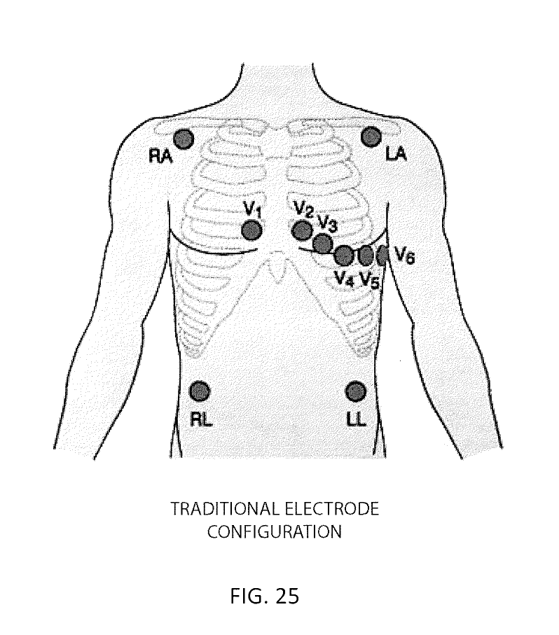

The locations of the leads (LA, RA, LL, RL and V1-V6) may correspond to the traditional 12-lead electrode placement, as illustrated in FIG. 25. The electrodes may be any of the electrodes described above, and may be integrated into the garment as discussed above (e.g., by screening, transfer, etc.). Additional sensors, and particularly motion and stretch sensors may be included. For example, in FIG. 23, the front of the garment shows five elongated stretch sensors 2319. These sensors may detect stretch in the body (as transmitted by stretching of the garment). The garment may also include one or more (e.g., three are shown in FIG. 24) motion sensors (e.g., IMUs). The sensors may be connected as described above (e.g., using an SMS, etc.).

In FIGS. 24-25 the garment is configured as sleeveless garment, which may also be configured as a tank-top. The neck region may be open (e.g., V-neck, C-neck, etc.). The garment may alternatively be configured as a short-sleeved or long-sleeved garment, and may be full-length (extending to or beyond the waist) or crop-topped (e.g., extending only to a position above a wearer's navel (e.g., approximately halfway down the user's torso). In any of these variations, the garment may be worn beneath a traditional outer garment such as a shirt, sweater, etc. The garment may include cutout regions around the wearer's armpits (e.g., beneath the sleeves, and down the sides of the body), preventing sweat or perspiration from collecting on the garment, allowing it to be worn longer without requiring washing. Any of these garments may be treated to prevent bacterial growth, etc., may include a coating or material that inhibits bacterial growth.

FIGS. 26A and 26B illustrate another example of a garment as described herein, configured to detect cardiac output (such as an electrocardiograph, ECG) and/or respiration. Any of the sensors described herein may be included. In any of these garments, the apparatus may include a side-opening and fastening, such as a zipper. In FIG. 26A, a zipper 2605 is located on the right side (to the wearer), which may aid in putting the garment on and taking the garment off. In this example, the chest electrodes 2607 (corresponding to leads V1-V6) are arranged across the chest portion of the garment so that they may be positioned against the traditional positions of the wearers chest when the garment is worn. The straps 2609 can be integrated into the garment (e.g., beneath the outer layer and/or between an inner and the outer layer). In FIG. 26A and 26B the garment straps 2605, 2605′ may be adjusted to secure the electrode contacts against the user's skin, as shown. Alternatively or additionally, the arm electrodes (LA, RA) 2611, 2611′ straps may be incorporated into the arms (in this example, near the bicep region) and one or more straps 2620, 2620′ may be used to hold the electrodes against the skin. FIG. 26B shows the back of the garment, and includes an adjustable buckle 2617, though other adjustable securements (e.g., Velcro, snaps, buttons, clamps, etc., may be used. In some variations, the securement may be positioned at the side or front of the garment. Leg electrodes (LL, RL) 2615, 2615′ are also integrated into the garment shown in FIG. 26B. As mentioned, the garment may include cut-out regions 2625, 2625′ that are positioned beneath the user's arms when the user is wearing the garment.

### Extended Wear Garments

Any of the garments described herein may be configured for extended wear (e.g., for more than single, daily use, including for longer-term/multi-day use without requiring washing). For example, described herein are extended-wear electronic extended-wear monitoring garments formed on a fabric (including compression garment fabrics) using a stretchable and conductive ink pattern along with monitoring components. The extended-wear monitoring garments may also have wireless control and communication capabilities. The electronic extended-wear monitoring garments contain features that allow it to be worn without frequent laundering.

The garments described herein may be used for monitoring a wearer's physiological condition over an extended periods and require less laundering. This may be useful in a number of scenarios. These extended-wear monitoring garments can be worn by athletes during training An extended-wear monitoring garment that absorbs less perspiration and/or prevents growth of microbes will result in a garment that will less likely chafe (from having the skin in contact with wet fabric) and can potentially be air dried without the unpleasant odors causes by axilla region being retained on the garment after it dries. An extended-wear monitoring garment may also require less changing for medical patients that require continuous monitoring but cannot be easily be moved.

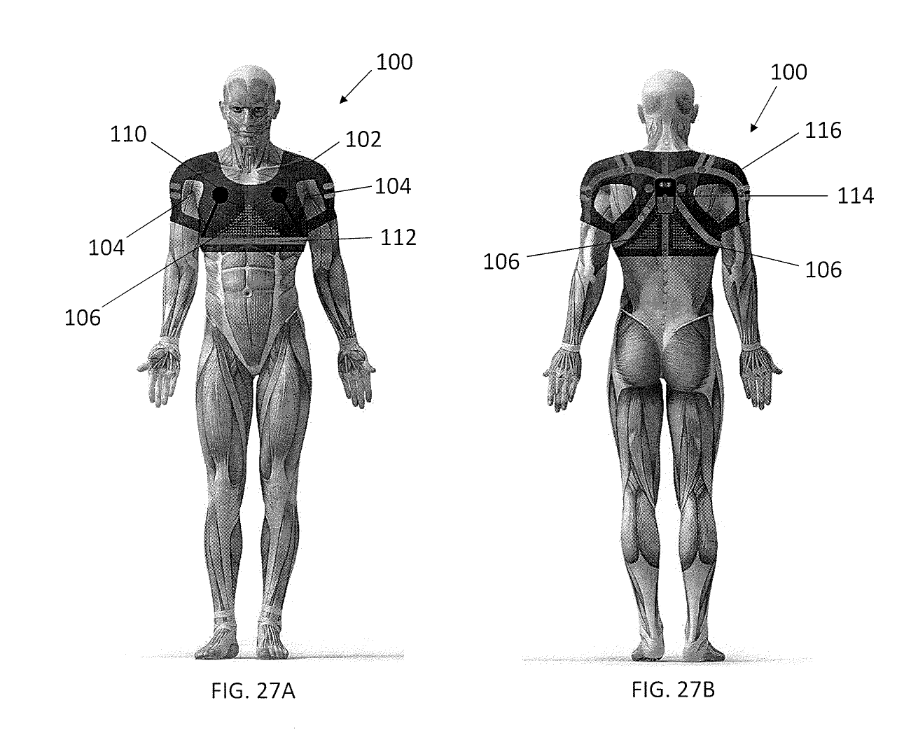

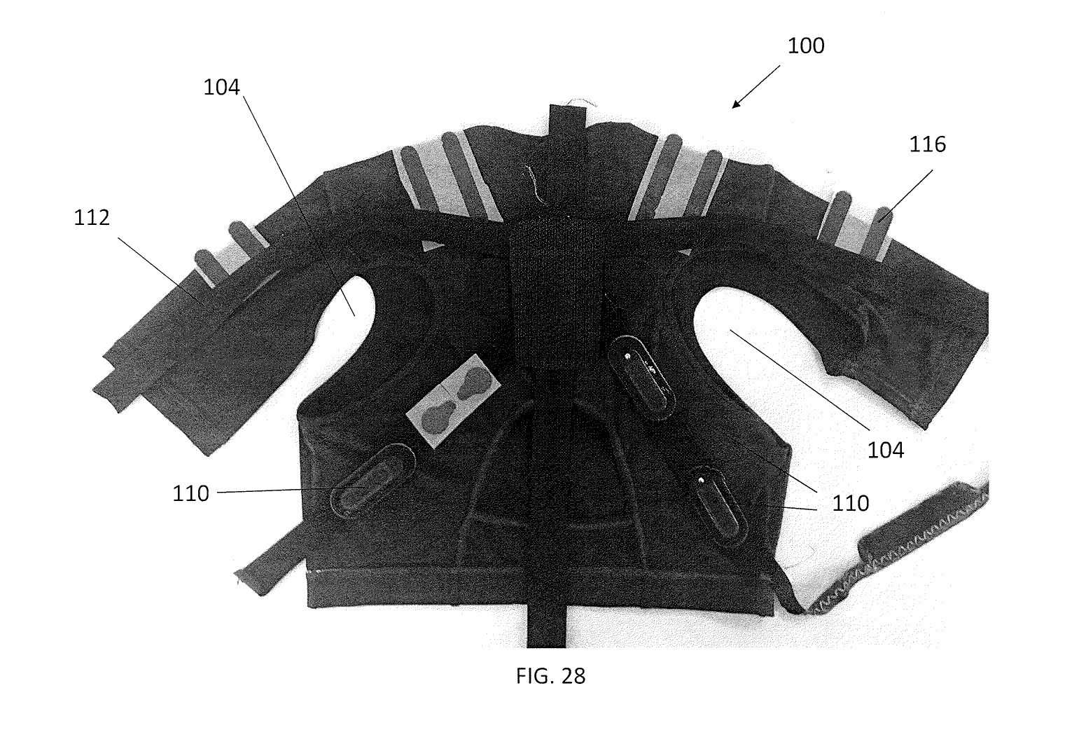

As mentioned, any of the garments described herein may include one or more features to enhance their long-term use (making them ‘extended-wear’ garments). An exemplary extended-wear monitoring garment, 100 is shown in FIGS. 27A and 27B (and FIG. 28), shown on a musculoskeletal model of a wearer. An extended-wear monitoring garment 100 may have a minimal profile. Decreasing the amount of fabric in contact with the wearer's skin may help minimize transfer of both perspiration and microbes onto the extended-wear monitoring garment and thus reduce odors associated with wearing a garment for an extended period. An extended-wear monitoring garment 100 may include a support material 102, cutouts 104, and breathable panels 106. Component-wise, an extended-wear monitoring garment 100 may contain at least one sensor 110, stretchable conductive traces 112 and/or sensors that can be used to detect a physiological parameter, a control module 114, and other conductive traces 116 for electrically connecting the sensors to the control module.

In FIG. 27A and 27B, support material 102 partially covers the torso of a wearer. Support material 102 may be tailored to provide coverage in the areas on a wearer where monitoring is desired and leave unencumbered areas of the wearer's body that do not provide any useful physiological signals. The support material may be straps, as discussed above, and may include an elastic material that can be adjusted by the wearer (user). As shown in FIGS. 27A and 27B, extended-wear monitoring garments 100 may extend only slightly past the pectoral region and terminates at the ribcage region on a wearer when worn. Truncating support structure 102 at this region maximizes the amount of monitoring that occurs on the wearer's torso (such as heart rate, and breathing) because the lower portion of the human torso does not typically provide useful, measurable physiological parameters. In addition, the absence of support material 102 for covering the lower torso may be beneficial in the athletic monitoring scenario because it limits the amount of wearer's perspiration that support material 102 will absorb, reduces the amount of restriction to the wearer's torso, allowing for more ventilation and circulation during physical activity.

The support material (including straps) may conform closely to the wearer's body and can be constructed from any suitable materials. Suitable materials are typically those that are lightweight so that the wearer does not feel obstructed during physical activity. Also, the support material should be breathable such that the wearer's excreted perspiration can quickly be wicked away from the wearer's skin and allowed to evaporate. In addition, it would be useful for the support material to be stretchable such that when the wearer moves the sensors associated with or contained within the extended-wear monitoring garment remains in contact with the wearer's skin in the correct position.

The support material of extended-wear monitoring garment may also include one or more super-breathable panels 106. Super-breathable panels 106 allow for more air flow to the corresponding areas on a wearer's skin that require more ventilation than other parts of the wearer's upper torso, but may also contain regions on the wearer's torso were monitoring may also be desirable or where it would be convenient to have connector elements that join various electronic components. In FIGS. 27A, 27B and 28, super-breathable panels 106 corresponds to areas just below the pectoral muscle and areas on the back just below the shoulder blades when worn. These areas of the human body contain mainly eccrine glands, still produce more perspiration than the shoulder and the actual pectoral regions. For a female form the super-breathable panels may be designed in the front to correspond to the regions under the breast region. Super-breathable panels 106 can be the same material as that of support material 102 but with additional ventilation apertures, or super-breathable panels 106 can be formed from a different material.

As mentioned, any of these extended-wear monitoring garment may include one or more cutouts 104. Cutouts 104 may correspond to the axillary regions on the wearer. Cutouts 104 allow for unobstructed ventilation of the axillary region, which may be beneficial for the wearer performing physical activities. Further, having no portion of extended-wear monitoring garment 100 come into contact with the axillary region while performing physical activities reduces transfer of perspiration and microbes from the wearer to extended-wear monitoring garment 100. Having less odor-causing perspiration and microbes absorbed onto extended-wear monitoring garment 100 may extend the wear-ability of extended-wear monitoring garment 100 between laundering.

The extended-wear monitoring garments described herein may be full length or partial length (as shown in FIGS. 27A-27B). Extended-wear monitoring garment 100 in this example includes at least one sensor, and in many cases, the extended-wear monitoring garment may contain a plurality of sensors, such as shown in FIGS. 23 and 24 and 26A-26B. The extended-wear monitoring garment may also include a central control module that is able to coordinate the sensor inputs and outputs, along with automatic and manual input and outputs from the wearer.

Control module 114 may have multiple functions. For one, control module 114 is able to communicate with the various sensors located on the extended-wear monitoring garment. Control module 114 may functions to record any physiological parameters detected by the sensors if a sensor management module is absent. Control module 114 can retain physiological parameters and communicate with external devices that then are used to analyze these parameters. The control module can be completely integrated into the extended-wear monitoring garment or partially integrated into the extended-wear monitoring garment. Connection between the control module and the various sensors can be based on a single 5-pin USB connection, thus substantially reduce the size of the female and male connectors from the device to the phone module. While the control module may be located in various regions of the extended-wear monitoring garment, it appears that in this case, where the extended-wear monitoring garment is truncated, the least obstructive location for the control module might be between the wearer's shoulder blades or within that vicinity.

The control module may be a module (chip) that manages the signals from and to the sensors, and may act as an interface between a communication device (sensor module configured from a phone, etc.) and sensors. The control module may manage the connection and interfaces between them. For example, and integrated control may include physical connections to sensors and may manage the way in which the signals are processed and sent between sensors and a sensor module and/or other analysis or control components. The control module may also include or may connect to a multiplexer to alternate readings between various sensors to which it is connected.

In some variations, the control module may provide proper power supply to passive sensors or active sensors. A control module may take power from the mobile systems through a port such as a USB port. An integrated control module may communicate from one side to a sensor module (e.g., communications systems/phone, etc. configured as a sensor module) through a USB port. The control module may act as an interface or a bridge between the sensors and the sensor module.

In addition, any of the integrated control modules described may be configured to include on-board processing (e.g., preprocessing), including, but not limited to: amplification, filtering, sampling (control of the sampling rate), and the like; typically basic pre-processing. An integrated control module may also encode signals from the one or more sensors. In some variations the control module may include a microcontroller on board. Further, and integrated control module may also generally manage communication protocols to/from any or all of the sensors, and may make an analog to digital conversion (if the signals are analog) and may also communicate with a comm port of a USB, before going to the USB. For example, the sensor management system or module may be configured to convert the signal into UART to the USB signal protocol.

In addition or alternatively, any of the integrated control modules may be configured as a signal receiver/transmitter. For example, an SMS integrated into the garment may be adapted to convert parallel signals to serial signals (in the order of the data).

As mentioned, an integrated control module may be placed in any position on a garment, but because of the reduced area on the extended-wear monitoring garment, an ideal location for the control module is on the back of the extended-wear monitoring garment between the shoulder blades. Although the control modules described herein are referred to as integrated, meaning they are integrated into the extended-wear monitoring garment, it may be possible for the control modules to be detachable from the extended-wear monitoring garment. It may be advantageous to be able to detach the control module prior to laundering because even if the control module is designed to be waterproof, reducing exposure to moisture and cleansing agents could extend the life of the control module and the extended-wear monitoring garment in general.

In some variations, the connectors (e.g., pins/ports) of the control module are adapted to water resistant/water proof. For example, the pins used may make connections that are waterproof, e.g., with connections that only open when you engage the male pin, but are otherwise closed and waterproofed.

In any of these integrated control modules, the control modules is a part of the garment, and are worn with the garment; the control modules may pre-process the signal(s) to prepare them for transfer.

The control module of the extended-wear monitoring garment may be positioned and permanently retained on the garment (onboard/dedicated), rather than separable from the garment, e.g., as part of a separate sensor module, such as a general-purpose smartphone that may be held in a pocket on the garment, as previously described. Extended-wear monitoring garment may have a microchip that allows the garment to have connectors (female and male) with a numbers of pins (inputs/outputs) so that data from all the sensors in the garment may be first processed by the sensor management system and then sent through a connection (e.g., as few as 1 or 2 pins, or more) to the phone/communication module. In general, some of the sensors and components of the garments described herein may individually require multiple connections and thus a dedicated signal management system may be very useful. For example, an IMU may require 5 pins and as many as 20 IMUs (or more) may be included as part of a garment, in addition to other sensors. Thus, the use of a dedicated sensor management system may allow the garment to manage a large number of data connections/contacts.

Extended-wear monitoring garment 100 contains sensors and sensor modules for detecting a plurality of inputs from the wearer. Potential sensors that can be incorporated into the extended-wear monitoring garment can include a heart-rate sensor, a respiration sensor and a skin conductance sensor. Such a sensor may be connected through a power trace to a module incorporated into the garment (such as placed between the scapulae). Data and other information may be managed by a sensor management system in the sensor module. Such data and other information may be sent, e.g. by the intelligent wear module, to the cloud in real time.

Sensors that are incorporated into the extended-wear monitoring garment should be flexible and able to conform to the contours of the wearer's form. In some instances, the extended-wear monitoring garment's support structure that sits adjacent to a sensor unit may be more resistant to deformation such that the support structure may more easily press the sensor unit against the wearer's skin.

In some examples, the sensor system of the extended-wear monitoring garment may be interactive. Some interactive sensors may be triggered by touch, by voice command or by proximity to a secondary object. An interactive sensor may, for example, comprise, a resistive touch point, a direct contact capacitive touch point, or a contactless touch points (through an outer garment). A touch point sensor includes two partial halves that form a complete circuit when the wearer touches the two regions and a small current is allowed to flow. Other sensors may be peripheral. A peripheral sensor (e.g. a sensor that is not part of the module such as a body sensor or interactive sensor, which sensors may be, for example an ink-based sensor or a traditional sensors, such as one implemented by an integrated circuit soldered on a rigid or flexible printed circuit board (PCBs)) may be connected to the smart module in any way. Such a connection may be, for example, made by a wire and/or a cable. Such a wire and/or cable may be fixed on the garment in any way, such as, for example, by: a) insulating ink embedding a conductive wire or a conductive cable (see description above) or by b) embedding a wire and/or a cable into a welded seam or into a seamless weld (e.g. may be smooth without an obvious join or seam), etc. A method of making a seamless weld with a trace may include overlapping two fabric portions, such as a compression polyester fabric, inserting a trace (e.g. such as a wire or cable) between the overlap and welding the fabric to connect the two fabric portions and thereby contain the trace inside the weld. A weld may be performed in any way, such as using heat to join the two fabric portions.

Extended-wear monitoring garment 100 may also include stretchable conductive traces 112. Stretchable conductive traces 112 can be incorporated into the extended-wear monitoring garment to detect a physiological change that can be correlated with a change in stretch of the conductive trace. Stretchable conductive traces can be used to measure a physiological parameter such as respiration around the ribcage. When a wearer inhales and exhales, the wearer's chest and ribcage expands and shrink, as a result, the resistance of the stretchable conductive trace changes in response to the expansion and reduction in circumference of the wearer's ribcage, which then is sensed by the sensor module and then can be sent to the control module or directly directed via the control module. Other feasible locations for using the stretchable conductive traces may be around the larger muscle group on or in the vicinity of the upper torso, shoulders, and upper arms, for measuring the amount of work or output from these muscle groups during physical activity.

Extended-wear monitoring garment 100 may also has flexible conductive traces 116 for joining the sensors, sensor modules with the control module. A conductive trace made from a conductive media may be made, for example from an insulating media embedding a layer of conductive material. Such a conductive trace may be used, for example, to bring a sensor condition and/or a power supply to a sensor or to an electrode (e.g. accelerometers, temperature sensor, etc.) so that a sensor and/or power supply may be placed in any location on the extended-wear monitoring garment; or it may be used to bring an electrical signal (e.g., variable current or voltage) from a sensor or electrode (e.g. an accelerometer, a temperature sensor, etc.) placed in any location on the shirt (e.g. on the arms) to the control module.

Extended-wear monitoring garment 100 may also include other communication components or interactive sensor systems not specifically shown in the figures. Interactive sensors can be touch point type sensors such that they allow a user to trigger a response, such as by proximity, by a touch, or by a voice command An interactive sensor may, for example, comprise, a resistive touch point, a direct contact capacitive touch point, or a contactless touch points (through an outer garment). A resistive touch point may be created, for example, by printing a plate of conductive media (ink), such as one which is formed by two apposed non-connected regions such as circles or in a comb-like pattern. By a simultaneous contact of the half-parts, the touch point (formed by the two apposed half parts) is closed to complete an electrical circuit is and a small electrical current is allowed to flow. Such a current may be generated by a voltage generator (such as one internal to a smart module). Such a current may travel from (or to) a smart module to a touch point via as a connecting trace such as one formed by a conductive ink media as described above). A plurality of such touch points may be placed in multiple sites on an intelligent wear garment item, such as on the outer part of the compression shirt. A touch point may include any shape and any size so long as a user is able to interact with it to generate an interactive sensor signal. In some embodiments, an apposed non-connected region may be less than 1 cm, from 1 cm to less than 3 cm, from 3 cm to less than 5 cm, from 5 cm to less than 7 cm, or may be greater than 7 cm in a longest dimension (such as a diameter). An interactive sensor may comprise a capacitive touch point. Such a capacitive touch points may be created in any way, such as proximity (e.g. a signal that may be travel through an outer garment such as by a finger coming close to the touch point).

A peripheral sensor (e.g. a sensor that is not part of the module such as a body sensor or interactive sensor, which sensors may be, for example an ink-based sensor or a traditional sensors, such as one implemented by an integrated circuit soldered on a rigid or flexible printed circuit board (PCBs)) may be connected to the smart module in any way. Such a connection may be, for example, made by a wire and/or a cable. Such a wire and/or cable may be fixed on the garment in any way, such as, for example, by: a) insulating ink embedding a conductive wire or a conductive cable (see description above) or by b) embedding a wire and/or a cable into a welded seam or into a seamless weld (e.g. may be smooth without an obvious join or seam), etc. A method of making a seamless weld with a trace may include overlapping two fabric portions, such as a compression polyester fabric, inserting a trace (e.g. such as a wire or cable) between the overlap and welding the fabric to connect the two fabric portions and thereby contain the trace inside the weld. A weld may be performed in any way, such as using heat to join the two fabric portions.

In general, extended-wear monitoring garment is designed to continuously hug and conform to the wearer's body when the garment is worn. In general, the flexible garment may include a first axis and a second axis perpendicular to the first axis wherein the garment is configured to stretch in size in the first axis but not to substantially stretch in the second axis. The conductive traces may extend substantially in one axis (e.g., in the second axis). Alternatively or additionally, the garment may be configured so that different regions of the garment are configured to stretch in a first direction but not in a second (substantially perpendicular) direction, or to not stretch in any direction; these different regions may be adjacent and the stretch vs. non-stretch regions may have different orientations, so that they do not all extend in the same axis relative to the garment. The conductive traces may extend substantially along the non-stretch directions of each region.

Finally, while extended-wear monitoring garment 100 as shown in FIGS. 27A and 27B has a scooped neck line, it is conceivable that the monitoring device may have any neck line shape. Further, the support material corresponding to the wearer's back can also have different configuration that what is currently shown. Sensor, sensor management modules, and the control module may be located in any suitable region on the extended-wear monitoring garment. While not shown, it is also conceivable that the extended-wear monitoring garment has closures such as buttons, snaps, Velcro, zippers, and so forth to aid in the putting on and taking off the extended-wear monitoring garment.

Uses for Extended-Wear Monitoring Garment

Extended-wear monitoring garment 100, as the name suggests, can be used to monitor a wearer's physiological parameter. While the extended-wear monitoring garment can be used in many different scenarios, the two most common situations are where the wearer uses the extended-wear monitoring garment during physical activity such as athletic training and conditioning, or where the extended-wear monitoring garment is used to for extended monitoring a patient, where moving the patient is difficult or untenable. In both such scenarios having an extended-wear monitoring garment provides a more comfortable experience for the wearer.

An intelligent garment or apparel system may include one or more than one intelligent sensor. A “power trace” (such as described elsewhere in the disclosure) may be used to supply power to a printed and/or physical sensor and/or a detector array strategically located on the apparel (“intelligent sensor”). Such a sensor may include a sensor that is not self-powered. Such a sensor may be configured to measure any of a host of physiological properties of the intelligent wear user that include but are not limited to: heart rate, respiratory rate, inspiratory time, expiratory time, tidal volume, rib cage contribution to tidal volume, perspiration, pulse, moisture, stress, glucose levels, pH balance, resistance, motion, temperature, impact, speed, cadence, proximity, movement, velocity, acceleration, posture, location, specific responses or reactions to a transdermal activation, electrical activity of multiple muscles (surface EMG), arterial oxygen saturation, muscle and tissue oxygenation in multiple sites, 36), oxyhemoglobin and/or deoxyhemoglobin concentration in multiple sites. “Smart sensor” may communicate with a smart module via wired or now known long range, medium range, and/or short range wireless application and communication protocols that include but are not limited to Bluetooth, FTP, GSM, Internet, IR, LAN, Near Field, RF, WAP, WiMAX, WLAN, WPAN, Wi-Fi, Wi-Fi Direct, Ultra Low Frequency, or hereafter devised wireless data communication systems, versions, and protocols for power and data communication and distribution; and may allow for all (or many) of the systems to work alone or together, and may be reverse compatible.

In some embodiments, by combining data from the sensors with input data from the extended-wear monitoring garment wearer, and with the additional input from a 3rd party, the extended-wear monitoring garment can build or continue to build a portfolio of knowledge on the extended-wear monitoring garment wearer, including, but not limited to, for example, peak heart rate after a certain activity or if abnormal physiological parameters are detected, then an emergency warning is sent to a wireless communication device (such as a smartphone) to send an emergency call for help which includes the wearer's name and location.

The extended-wear monitoring garment according to the disclosure may include providing, developing and/or creating software applications, mobile device applications, and hardware applications; providing, developing and/or creating soft-goods (such as a textile, a fabric, an apparel merchandise); and/or hard-goods (such as an exercise equipment, wrist band, etc.). Such an application may be utilized to create visual, audio and/or tactile effects that may be controllable by the user of such applications such as on soft-goods or hard-goods. Such applications may be used in order to sense, read, analyze, respond, communicate and/or exchange content/data feedback with the user. Any type of communication protocol may be used, such as in conjunction with the internet, attached or separate mobile devices, and other communication tools.

Specialized location based elements and tracking components, inks, Nano formulations, conductive materials, component transmitters, analysis and artificial intelligence response software and hardware, receivers, low, no, and high powered sensors, printed speakers, connectors, Bluetooth and USB functions, energy generating elements, medical and wellness tracking and feedback devices, body movement and efficiencies and mechanisms for tracking and analyzing the same, and other such like elements, alone or in conjunction with each other may be utilized in an intelligent wear system.

### Monitoring Respiration

Any of the monitoring garments described herein may be adapted to detect respiration, and in particular, regional respiration. Such devices may be used at the request of a medical professional, or by anyone who wishes to monitor respiration. A respiration-monitoring device may be adapted for the continuous and accurate monitoring of respiration, including monitoring of respiration in one or more regions. A complete and accurate measurement of several respiratory parameters (described below) may be made using a plurality of stretchable conductive ink traces (patterns) arranged in a wavy pattern (e.g., a ‘zig-zag’ pattern, a sinusoidal pattern, saw tooth pattern, etc.) arranged in different regions of the garment so that they are positioned about a wearer's torso. Regions including lengths of stretchable conductive ink may include: the anterior (front) part of a shirt, the posterior (back) part of a shirt; each or either of the two lateral sides of a shirt, etc. Sub-regions within these regions may also be used (e.g., upper/lower, left/right, etc.). The stretchable conductive ink, as described above, may have a resistance that varies slightly with stretch; this property may be used to detect and/or measure body movement as the ink is stretched while worn on the body.

In general, conductive ink traces may be used as sensor. The sensor can be a plurality of conductive ink traces that are stretchable traces. Any of these devices may also include a sensor manager unit. The sensor management unit may be a processor that is placed on the garment (e.g., on the back) in connection with an interface for connecting the sensors to the processor. The processor may be, for example, a smartphone or other handheld device. The apparatus may have a communication unit; this communication unit may be separate or may be integrated with the processor (and/or may include its own dedicated processor). For example, a communication unit may also be placed on the back, and connect to the interface. Additional sensors may also be used, including motion sensors. For example, a tri-axes accelerometer (alone or, e.g., embedded in the communication system), may be included.

In general, any of these devices may include one or more wearer inputs, such as ‘touchpoint sensors’. For example, capacitive touch points may be used. A touchpoint sensor may include two electrodes (e.g., one on the inner, the other on the outer, surface of the garment in corresponding positions), made of conductive ink patterns, a separating layer of the textile between the two conductive electrode patterns; and an insulating layer deposited onto the internal conductive ink pattern layer. A connecting trace may be included between the external electrode and a terminal point placed close to the neck.

In general, the respiratory traces may be positioned in any region of the body of the shirt to detect movement (expansion/retraction) due to respiration in that portion of the body. A complete and accurate measurement of several respiratory parameters (see below) may be provided for individual regions of the wearer's body by positioning stretchable conductive ink traces, ‘zig-zag’ shaped, (e.g., by transfer process) in different regions of the body of the shirt. For example, conductive traces may be positioned on the anterior and the two lateral sides of the shirt, on the posterior part (back) of the shirt, and in various sub-regions of these portions.

### Heart Monitoring

Also described herein are garments that may be used to effectively and continuously monitor electrocardiogram (ECG) signals. For example, a garment may be adapted to measure signals by including pairs of redundant traces between which the apparatus (e.g., garment, control/sensing module, etc.) may switch. In some variations the sensor management system and/or a sensor module may determine which set of electrodes between the redundant multiple electrodes to use in detecting a particular lead for an ECG. The electrodes used to detect ECG signals may be formed of the stretchable conductive ink composites described herein. In some variations, the electrodes are printed, applied or formed on one side of the garment (e.g., the inner surface) and adapted to be in continuous contact with the subject's skin so as to measure ECG signals. Electrodes may be connected via conductive traces (formed by, for example, stretchable conductive ink patterns and/or combinations of stretchable conductive ink patterns and higher-conductance traces such as conductive thread and/or printed Kapton, or just formed of a higher-conductance trace such as a conductive thread and/or printed Kapton) to an SMS and/or sensor module. The SMS and/or sensor module may determine, e.g., based on the quality of the signal, which of the redundant traces to use/present for the ECG signal.

In any of the ECG-sensing garments, the electrodes may be held against the body for consistent/constant measurement (even during motion) by the structure of the garment, including by an additional harness region. This harness may be formed as a region supporting the ECG chest electrodes that is relatively more supportive (e.g., applying pressure/force) to hold the chest electrodes on/against the body, even during respiration and other body movements. For example, the harness region may be formed as an elastic corset (e.g., width: 2 cm on the sternum, 4 cm on the xiphoid line) running along the sternal line, then separating on the right and left sides of the xiphoid line, then on the back, then converging on the spinal cord and running up to the neck, then again separating into right and left sides around the neck, to finally converging on the sternal line. The material of the corset has to be extremely extensible.

The electrodes, and/or the region peripheral to the (e.g., chest) electrodes may include a silicone surface that helps hold the electrode(s) against the chest, and may also prevent the electrodes from slipping. For example, silicone may be located in an inner surface of the shirt, corresponding to the harness/corset position, along the horizontal line on both sides up to 5 cm beyond the midaxillary lines. This silicone may help ensure that the ink electrodes are fixed to the chosen position and do not move with patient's motion.

As mentioned, it is particularly helpful that the electrode include adjacent redundant electrodes. All of the electrodes (including the redundant electrodes) may be connected to the SMS and/or control module to detect ECG signals and the SMS and/or control module may decide which of the redundant signals to use (or in some variations to use the redundant signals to improve the overall signal quality, e.g., by selective filtering, averaging, or the like). In some variations the non-selected redundant signal may be ignored; in other variations the apparatus may be configured to store it for later analysis. Electrodes may have signals that may be stored, transmitted and/or processed; decisions about which of the redundant electrodes to use to generate an ECG may be made later. Body temperature monitoring

Any of the garments described herein may include sensors that detect other physiological parameters in addition to respiration and heart beat and rhythms. For example, extended-wear monitoring garments described herein may include sensors for detecting a wearer's body temperature. In the physical activity monitoring scenario, the extended-wear monitoring garment may warn the wearer if a set critical temperature is reached via a signal or alarm. The signal or alarm will help the wearer from overheating and suffering from heat exhaustion during physical activity and indicate to the wearer that it may be time to pause for hydration or to seek cover if it is an inordinately hot day.

### Wireless Communication

Any of the monitoring garments described herein may be able to communicate with external devices. As previously mentioned, an app on a smartphone or on a tablet can be used to program the control module of the extended-wear monitoring garment such that the appropriate sensors will take a physiological reading during a pre-determined time. In addition to manual input, the extended-wear monitoring garment may respond to audio commands from the wearer. Extended-wear monitoring garment may also have the capability to communicate with a corresponding smart phone through the app in case of emergencies where the wearer requires assistance or medical attention.

### EXAMPLES

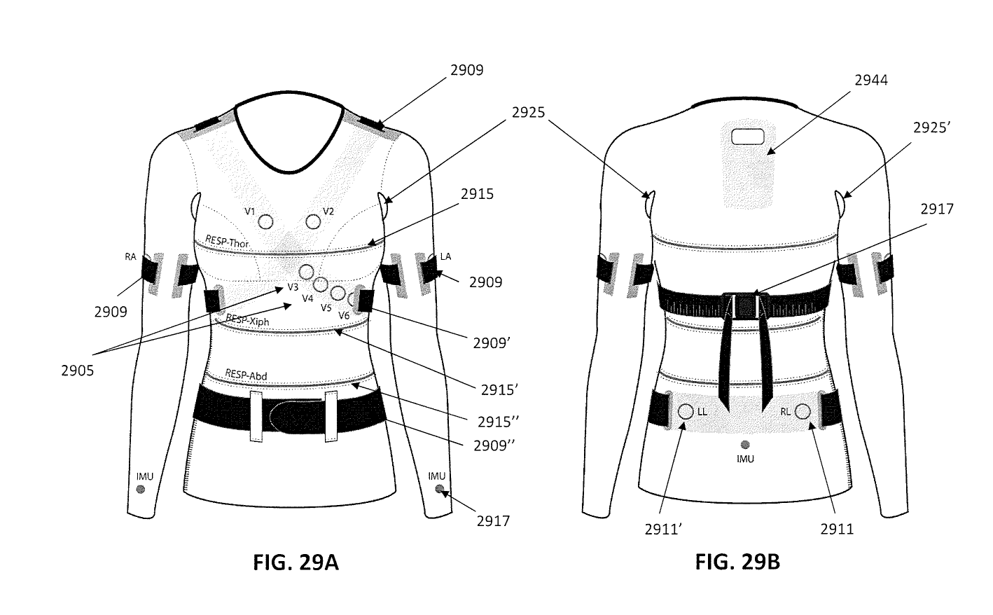

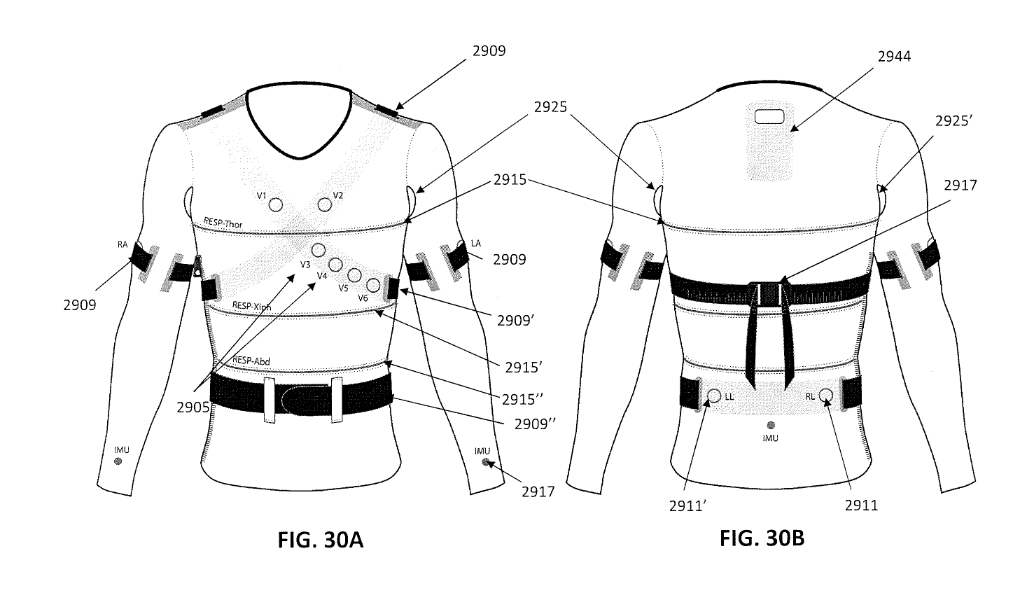

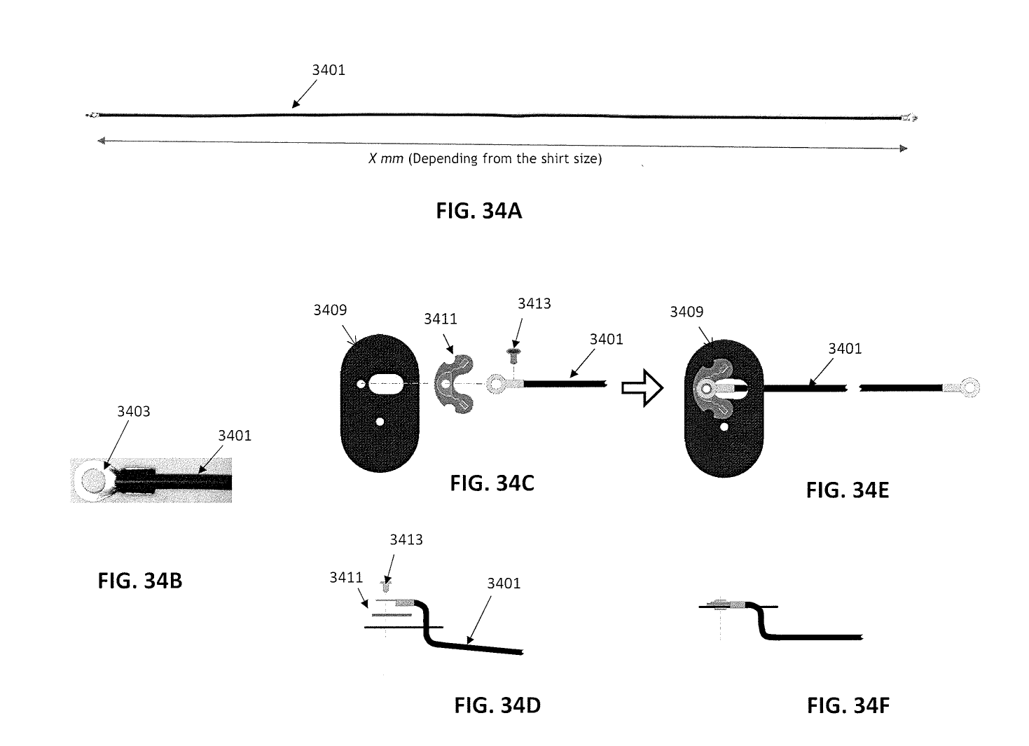

FIGS. 29A-29B and 30A-30B show another example of a garment as described herein, similar to that shown above in FIGS. 26A and 26B. In FIGS. 29A-29B and 30A-30B the garment is configured to detect both respiration and cardiac output (such as an electrocardiograph, ECG). Any of the sensors described herein may be included. In any of these garments, the apparatus may include a side-opening, a front opening or a back opening and fastener, such as a zipper or Velcro closure (not shown in FIG. 30A-30B). Chest electrodes 2905 (corresponding to and labeled as leads V1-V6) are arranged across the chest portion of the garment so that they may be positioned against the traditional positions of the wearers chest when the garment is worn. Right and left arm electrodes (RA, LA) are also show on the arms. Straps 2909 can be integrated into the garment (e.g., beneath the outer layer and/or between an inner and the outer layer), e.g., on the arms and across the chest and back 2909′ and/or waist 2909″. Additional sensors for use with ECG detection, e.g., right leg (RL) 2911 and left leg (LL) 2911′ are shown on the lower back of the garment. The garment shown also includes three horizontally-arranged respiration sensors 2915, 2915′, 2915″ formed from a silicone conductive cord (as shown in greater detail in FIGS. 34A-34F, described below. In FIGS. 29A-29B and 30A-30B, IMU (inertial sensors) 2917 are also shown positioned, in this example on the arms near the writs and the back. Differential comparison between these different inertial sensors may provide an indication of the wearer's body position, and relative arm position.

In FIG. 29A-29B and 30A-30B the garment straps 2909, 2909′, 2909″ may be adjusted to secure the electrode contacts against the user's skin, as shown. The straps may be incorporated into the garment partially (as shown in this example) or completely. The backs of the garments (shown in FIG. 29B and 30B) includes an adjustable buckle 2917, though other adjustable securements (e.g., Velcro, snaps, buttons, clamps, etc., may be used. In some variations, the securement may be positioned at the side or front of the garment. As mentioned, the garment may include cut-out regions 2925, 2695′ that are positioned beneath the user's arms when the user is wearing the garment.

In FIGS. 29A-29B and 30A-30B, the respiration sensors comprises three parallel lines of cord running completely around the torso at different heights, upper chest, diaphragm and navel. As shown below in reference to FIG. 34A-34I the conductive cord may be connected between the signal and ground ends on the garment and the change in conductance (or resistance) of the cord may be related to respiration. For example, in FIG. 34A, a length of electrically conductive cord 3401 (e.g., a cord or tube formed of a conductive silicone rubber that includes an outer insulator) is shown. A connector (e.g., ring terminal) may be placed on either rend of the length. The length x may be cut to a predetermined length based on the size of the garment, and the terminals 3403 attached to either end (e.g., by crimping, etc.). The conductive cord may then be coupled to a support 3409 (with or without a rigid or semi-rigid substrate 3411, as shown in FIGS. 34C (and side view 34D) and 34E (and side view 34F). A connector 3413 (e.g. rivet) may be used to connect the conductive cord ends to the limiter support (substrate 3409). The other end may be pulled through a channel (e.g., a fabric channel that is attached or to be attached to the garment. The attachment end may then be coupled to the framework (e.g., FIG. 57) and an electrical contact made through the connector. See, FIG. 34G. The opposite end may then be connected to a support substrate and connector, as shown in FIG. 34H, and attached to the opposite strip (e.g., 5725′ in FIG. 57), as shown in FIG. 34I. The connections may be sealed (and insulated). A fabric cover (sensor cover) may also be applied over the ends.

In operation, the electrically conductive cord may provide a stretch sensor that changes an electrical property (e.g., resistance) with stretch within a relatively linear range that provides a sufficient measure of respiration based on the stretching and relaxation of the cord during breathing while wearing the garment. The electrically conductive cord may include conductive carbon fibers. Thus, by monitoring the cyclic changes in electrical properties through the cord, e.g., resistance, during breathing, the user's respiration may be monitored at the three or more different levels of the body. Motion artifacts may be detected using the other sensors (e.g., IMUs) and subtracted from the sensor output.

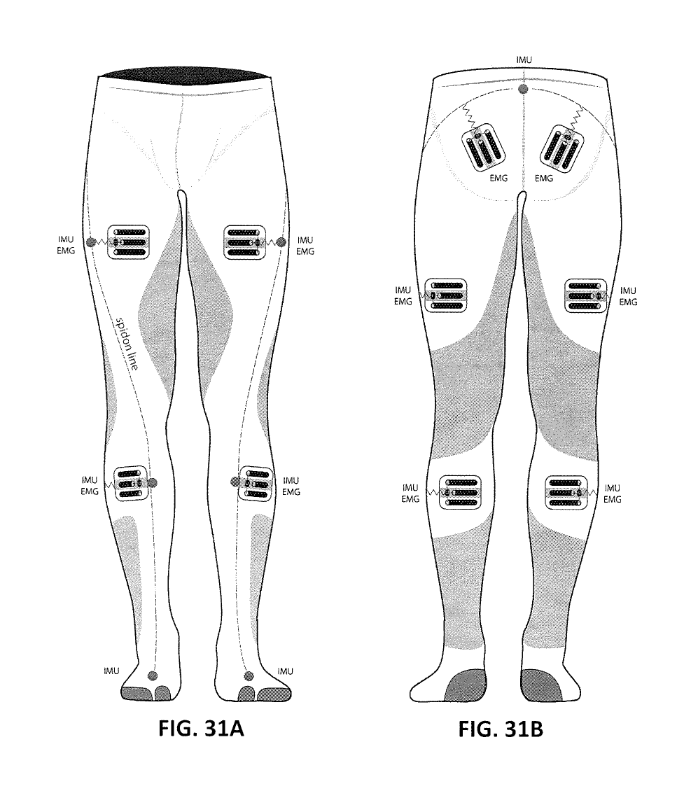

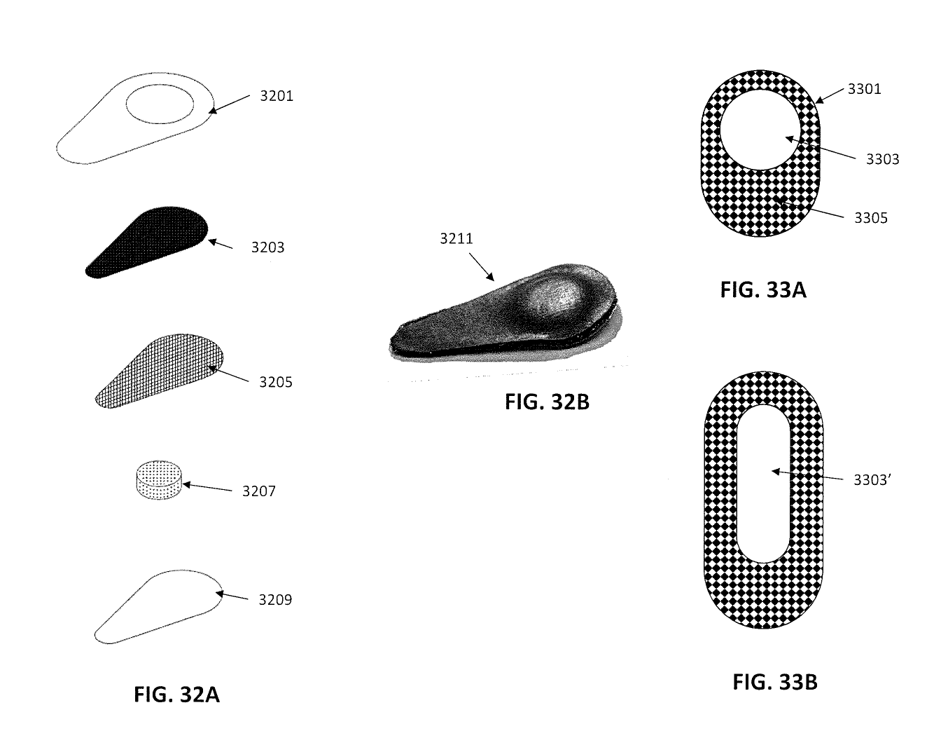

In general, any of the electrodes for contacting the wearer's body described herein may be configured as an assembly of electrodes. For example, FIG. 32A-32B illustrate an embossed electrode configured for EEG, EOG, EMG and/or ECG that includes an expandable (e.g., self-expanding, foam support that may be expandable and compressible). In FIG. 32A the electrode assembly is shown in an exploded view including an electrode cover 3201, a conductive ink 3203, an electrode support 3205 material (fabric), a compressible contact support 3207 (shown here as a sponge foam for increased contact to skin and therefore conductance). And an electrical limiter (e.g., insulator) 3209. FIG. 32B shows an example of a partially assembled electrode 3211, without a cover. Thus, in FIGS. 32A-32B the electrode assembly includes a self-inflating (e.g., foam 3207) element that is supported by a support (cover 3201), which helps bias or push the electrode against the body. In conjunction with the straps forming another portion of the supporting frame in the garment, the device may securely, comfortably and flexibly hold the electrodes against the subject's body. The self-inflating or foam supports 3207 may be sized to match the size of the electrode (e.g., coextensive with them) or they may smaller (sub-extensive) or larger.

Any appropriate covers may be used. The electrode cover may be particularly helpful to keep the electrode in contact with the wearer's skin. For example, FIGS. 33A and 33B illustrate electrode covers that are configured to include a grip pattern on the surface. In this example, the electrode covers are formed of a fabric material onto which a pattern of micro protrusions (e.g., balls, spheres, etc.) of a high-grip material such as silicone or polyurethane material has been applied by an extrusion or print process. These micro balls may help assure a good adesion of the electrode to the skin in order to have a stable biometric signal. This pattern may also allow the sweat to drain from the skin surface (e.g., a pattern having gaps, rows, columns, etc.) as shown. The gaps may be spaced to be >0.1 mm (e.g., 0.1 mm or more, 0.2 mm or more, 0.3 mm or more, 0.5 mm or more 1 mm or more, etc.) apart. In FIG. 33A, the fabric support 3301 is a soft fabric support with an adhesive on (or may be applied onto) one side. The cover includes a window 3303. As mentioned, the plurality of grip protrusions 3305 may be attached or formed onto the skin-facing surface. FIG. 33B shows a similar variation with a larger window or opening for the electrode.

FIGS. 31A and 31B illustrate pants (e.g., leggings, tights, etc.) that may be worn and include one or more sensors for detection of physiological state or performance. In FIGS. 31A and 31B, a plurality of sensors (IMU and EMG sensors) are positioned at various locations around the user's body. The sensor output may be coupled to the shirt (which may hold a controller (e.g., phone and/or dedicated processor) 2944.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A garment for monitoring electrical signals from a wearer's skin, the garment system comprising:
a garment body formed of a flexible first fabric, wherein the garment body is configured to be worn on a torso;
a set of electrical sensors arranged at discrete locations on the garment body when the garment is worn, wherein each electrical sensor is positioned on an inner surface of the garment body;
a channel within the garment body;
a strap within the channel, wherein a portion of the strap is exposed from an outer surface of the garment when worn;
wherein the strap overlies at least some of the electrical sensors; and
a plurality of discrete expandable supports, wherein each expandable support is between the strap and one of the electrical sensors of the first set of electrical sensors, further wherein each expandable support is configured to expand and compress in an axis perpendicular to the electrical sensors.

2. The garment of claim 1, wherein the garment is adapted to sense ECG signals, patient movement, and torso stretch.

3. The garment of claim 1, wherein the garment is configured as a long-sleeve shirt.

4. The garment of claim 1, wherein the set of electrical sensors comprises ECG sensors arranged with each sensor having a sensing surface exposed on an inner surface of the garment.

5. The garment of claim 1, wherein the strap crosses a front portion of the garment in an X-pattern.

6. The garment of claim 1, wherein the strap comprises two branches, wherein the first branch overlies one or more electrodes and the second branch overlies one or more electrodes.

7. The garment of claim 1, wherein the garment further comprises a second strap within a channel near a waist region of the garment, wherein the second strap is adjustable.

8. The garment of claim 1, wherein the first strap is an elastic strap.

9. The garment of claim 1, further comprising a plurality of stretch sensors integrated into the garment.

10. The garment of claim 9, wherein the stretch sensors each comprises a silicone conductive cord.

11. The garment of claim 1, wherein each electrical sensor comprises a flexible conductive ink electrode printed on an inner surface of the garment body.

12. The garment of claim 1, further comprising a left side and a right side cut-out corresponding to a left and right axillary region of the wearer that is configured to be positioned at the wearer's armpits when the garment is worn.

13. A garment system for monitoring electrical signals from a wearer's skin, the garment system comprising:
a garment body formed of a flexible first fabric, wherein the garment body is configured to be worn on a torso;
a set of electrical sensors arranged at discrete locations on the garment body when the garment is worn, wherein each electrical sensor is on an inner surface of the garment body;
a first strap extending in an X-shaped pattern over a front of the garment;
a channel within the garment body, wherein the first strap extends within the channel; further wherein the strap is adjustable through a portion of the first strap that is exposed from the channel on an outer surface of the garment;
a second strap extending around a waist portion of the garment beneath the first strap, wherein the second strap extends within a second channel within the garment body;
wherein the first strap overlies the set of electrode; and
a plurality of discrete expandable supports, wherein each expandable support is between the first strap and one of the electrical sensors of the set of electrical sensors, further wherein each expandable support is configured to expand and compress in an axis perpendicular to the electrical sensors.

14. The garment of claim 13, wherein the set of electrodes comprises a V1, V2, V3, V4, V5, V6, LA, LL, RA and RL electrodes configured to sense electrocardiogram (ECG) data, further wherein the first strap overlies the V1, V2, V3, V4, V5, V6, LA, and RA electrode and the second strap overlies the LL and RL electrodes.

15. The garment of claim 13, wherein the first strap is an elastic strap.

16. The garment of claim 13, further comprising a plurality of stretch sensors integrated into the garment.

17. The garment of claim 15, wherein the stretch sensors each comprises a silicone conductive cord.

18. The garment of claim 13, wherein each electrical sensor comprises a flexible conductive ink electrode printed on an inner surface of the garment body.

19. The garment of claim 13, further comprising a left side and a right side cut-out corresponding to a left and right axillary region of the wearer that is configured to be positioned at the wearer's armpits when the garment is worn.

20. A garment system for monitoring electrical signals from a wearer's skin, the garment system comprising:
a garment body formed of a flexible first fabric, wherein the garment body is configured to be worn on a torso;
a set of electrical sensors arranged at discrete locations on the garment body when the garment is worn, wherein each electrical sensor comprises a flexible conductive ink electrode printed on an inner surface of the garment body;
further wherein the set of electrodes comprises V1, V2, V3, V4, V5, V6, LA, LL, RA and RL electrodes that are configured to sense electrocardiogram (ECG) data;
a first strap extending in an X-shaped pattern over a front of the garment;
a channel within the garment body, wherein the first strap extends within the channel; further wherein the strap is adjustable through a portion of the first strap that is exposed from the channel on an outer surface of the garment;
a second strap extending around a waist portion of the garment beneath the first strap, wherein the second strap extends within a second channel within the garment body;
wherein the first strap overlies the V1, V2, V3, V4, V5, V6, LA, and RA electrode and the second strap overlies the LL and RL electrodes; and
a plurality of discrete expandable supports, wherein each expandable support is between the strap and one of the electrical sensors of the first set of electrical sensors, further wherein each expandable support is configured to expand and compress in an axis perpendicular to the electrical sensors.
